(19) United States
(12) Patent Application Publication
(10) Pub. No.: US 2023/0365714 A1
(43) Pub. Date: Nov. 16, 2023

(54) ANTIBODIES CAPABLE OF BINDING TO ROR2 AND BISPECIFIC ANTIBODIES BINDING TO ROR2 AND CD3

(71) Applicant: GENMAB A/S, Copenhagen V (DK)

(72) Inventors: Louise Koopman, Utrecht (NL); Patrick ENGELBERTS, Amersffoort (NL); David SATIJN, Utrecht (NL); Jan Hermen DANNENBERG, Utrecht (NL)

(21) Appl. No.: 18/029,472

(22) PCT Filed: Oct. 1, 2021
(86) PCT No.: PCT/EP2021/077130

§ 371 Date: Mar. 30, 2023

(30) Foreign Application Priority Data
Oct. 2, 2020 (EP) 20199893.7

(51) Int. Cl. C07K 16/46; A61P 35/00

(52) U.S. Cl. CPC C07K 16/468; A61P 35/00; C07K 2317/31; C07K 2317/565; C07K 2317/24; C07K 2317/35; C07K 2317/92

(57) ABSTRACT

The present invention relates to antibodies binding to ROR2, including bispecific antibodies binding to ROR2 and CD3. The invention further provides pharmaceutical compositions comprising the antibodies and use of the antibodies for therapeutic and diagnostic procedures, in particular in cancer therapy.

### FIELD OF THE INVENTION

The present invention relates to antibodies binding to ROR2, including bispecific antibodies binding to ROR2 and CD3. The invention further provides pharmaceutical compositions comprising the antibodies and use of the antibodies for therapeutic and diagnostic procedures, in particular in cancer therapy.

### BACKGROUND

ROR2 (receptor tyrosine kinase-like orphan receptor 2, NTRKR2, neurotrophic tyrosine kinase receptor-related 2), is a single-pass type I transmembrane glycoprotein that belongs to the ROR subfamily of the tyrosine protein kinase family. ROR2 is a tyrosine kinase receptor important in regulating skeletal and neuronal development, cell migration and cell polarity, in part via its proposed role in the non-canonical Wnt5a signaling pathway (Oishi 2003, Genes to cells 8:6450654.) It contains a FZ (frizzled) domain, an Ig (immunoglobulin)-like C2-type domain, and a kringle domain in the extracellular region and a protein kinase domain in the cytoplasmic region (Masiakowski and Carroll 1992, J Biol Chem 267:26181-90).

In normal human adult tissue, ROR2 expression is very limited (only in uterus during menstrual cycle, in brain during repair upon damage, in bone during bone formation, and in gut as part of intestinal homeostasis (Debebe and Rathmell 2015, Pharmcol & Therap 150:143-148; Endo 2017, Dev Dyn 247:24-32), whereas ROR2 expression is found on human tumor cells in numerous cancer tissues, including sarcoma, uterine, pancreas, melanoma, renal cell carcinoma, prostate carcinoma, colorectal cancer, squamous cell carcinomas of the head and neck, stromal tumors and breast cancer tissue (reviewed in Debebe and Rathmell 2015, Pharmcol & Therap 150:143-148).

Hence, targeting of ROR2 has been proposed for the treatment of cancer. For example, a ROR2-specific antibody drug conjugate CAB-ROR2-ADC/BA3021 is in development for cancer therapy in solid tumors and soft tissue sarcoma (Sharp et al. Proceedings of the AACR Annual Meeting 2018; Cancer Res 78(13 Suppl): abstract 833). Also, chimeric antigen receptor (CAR) T cells directed to ROR2 are in development in kidney cancer (Association for Cancer Immunotherapy (CIMT) 2019 Annual Meeting, abstract 123).

Efforts to target T cells to ROR2 have also been made. A ROR2/CD3 bispecific in scFv-Fc format, based on a humanized ROR2 rabbit antibody has been described, displaying T cell cytotoxicy in tumor cell lines (Goydel et al 2020, J Biol Chem 295:5995-6006).

While some progress has been made, there is still a need for the development of antibody-based cancer therapies targeting ROR2 that are efficacious and safe for human use such as for use in the treatment of cancer.

It is an object of the present invention to provide an antibody comprising at least one antigen-binding region capable of binding to human ROR2. It is a further object of the invention to provide an antibody comprising two antigen-binding regions capable of binding to human ROR2. It is a further object to provide a bispecific antibody capable of binding to human ROR2 and human CD3, such as human CD3ε (epsilon). It is a further object to provide a CD3×ROR2 bispecific antibody of IgG format such as human IgG1 format. It is a further object to provide a CD3×ROR2 bispecific antibody of IgG1 format where the Fc region is inert. It is a further object to provide a CD3×ROR2 bispecific antibody that has a plasma half-life in the range of a regular human IgG1 antibody. It is a further object to provide a ROR2 antibody and/or a CD3×ROR2 bispecific antibody that is efficacious and safe for the treatment of cancer.

### SUMMARY OF INVENTION

In a main aspect, the present invention relates to ROR2 binding antibodies and in particular to an antibody comprising at least one antigen-binding region capable of binding to human ROR2 wherein said antibody comprises a heavy chain variable (VH) region CDR1, CDR2, and CDR3 having the sequences as set forth in SEQ ID NOs: 3, 4, and 5, respectively, and a light chain variable (VL) region CDR1, CDR2, and CDR3 having the sequences as set forth in SEQ ID NO:7, 8 and 9, respectively.

In a further aspect, the antibody may in particular be a bispecific antibody comprising a first antigen binding region capable of binding human ROR2 wherein said antibody comprises a VH region CDR1, CDR2, and CDR3 having the sequences as set forth in SEQ ID NOs: 3, 4, and 5, respectively, and a VL region CDR1, CDR2, and CDR3 having the sequences as set forth in SEQ ID NO:7, 8 and 9, respectively, and comprising a second antigen binding region capable of binding to human CD3 such as human CD3ε (epsilon), such as human CD3ε (epsilon) as specified in SEQ ID NO: 21.

In a further aspect, the present invention relates to a bispecific antibody comprising a first antigen binding region capable of binding human ROR2 as described herein and a second antigen binding region capable of binding to human CD3 comprising a VH region CDR1, CDR2, and CDR3 having the sequences as set forth in SEQ ID NOs: 23, 24, and 25, respectively, and a VL region CDR1, CDR2, and CDR3 having the sequences as set forth in SEQ ID NO:27, GTN and 28, respectively.

In another aspect, the present invention relates to a nucleic acid construct comprising
a) a nucleic acid sequence encoding a heavy chain sequence of an antibody comprising an antigen-binding region capable of binding to ROR2 as defined herein, and/or
b) a nucleic acid sequence encoding a light chain sequence of an antibody comprising an antigen-binding region capable of binding to ROR2 as defined herein.

In another aspect, the present invention relates to an expression vector comprising
a) a nucleic acid sequence encoding a heavy chain sequence of an antibody comprising an antigen-binding region capable of binding to ROR2 as defined herein, and/or
b) a nucleic acid sequence encoding a light chain sequence of an antibody comprising an antigen-binding region capable of binding to ROR2 as defined herein.

In another aspect, the present invention relates to a cell comprising a nucleic acid construct or an expression vector as defined herein.

In a further aspect, the present invention relates to a composition comprising an antibody according to any aspect or embodiment herein.

In a further aspect, the present invention relates to a pharmaceutical composition comprising an antibody according to any aspect or embodiment herein and a pharmaceutically acceptable carrier.

In another aspect, the present invention relates to an antibody according to any aspect or embodiment herein for use as a medicament, such as for use in the treatment of a disease.

In a further aspect, the present invention relates to a method of treating a disease or disorder, the method comprising administering an antibody, a composition or pharmaceutical composition according to any aspect or embodiment herein, to a subject in need thereof.

In one aspect, the invention relates to a method of producing an antibody according to any aspect or embodiment herein, comprising cultivating a recombinant host cell in a culture medium and under conditions suitable for producing the antibody.

In another aspect, the present invention relates to a kit-of-parts, comprising an antibody as defined herein; and instructions for use of said kit.

These and other aspects and embodiments of the invention are described in more detail below.

### BRIEF DESCRIPTION OF THE DRAWINGS

FIG. 1. Binding to ROR2 expressed on the cervical cancer cell line HeLa of rabbit-human chimeric antibody chIgG1-ROR2-A-FEAR and humanized variants hereof according to the invention, determined by flow cytometry.

FIG. 2: Binding of bispecific CD3×ROR2 antibodies and monospecific ROR2 antibodies of the invention to CHO cells expressing human or cynomolgus monkey ROR2. Binding of bsIgG1-huCD3-FEAL×chROR2-A-FEAR, bsIgG1-huCD3-FEAL×ROR2-A-HC4LC3-FEAR, chIgG1-ROR2-A-FEAR and IgG1-ROR2-A-HC4LC3-FEAR to CHO cells expressing human ROR2 (left panels) or cynomolgus monkey ROR2 (right panels) was determined by flow cytometry. Untransfected CHO cells were used as negative control (not shown).

FIG. 3. Binding of ROR2 monospecific and CD3×ROR2 bispecific antibodies of the invention to CHO cells expressing human, cynomolgus monkey ROR2 or a T322M variant of cynomolgus monkey ROR2. Binding of chIgG1-ROR2-A-FEAR and bsIgG1-huCD3-FEAL×chROR2-A-FEAR was determined by flow-cytometry.

FIG. 4. Binding of CD3×ROR2 bispecific antibodies according to the invention to CHO cells expressing a T322M variant of cynomolgus monkey ROR2. Binding of bsIgG1-huCD3-FEAL×chROR2-A-FEAR, bsIgG1-huCD3-FEAL×ROR2-A-HC4LC3-FEAR, bsIgG1-huCD3-H101G-FEAL×chROR2-A-FEAR and bsIgG1-huCD3-H101G-FEAL×ROR2-A-HC4LC3-FEAR to CHO cells expressing RORmf-T322M was determined by flow-cytometry.

FIG. 5. Binding of CD3×ROR2 bispecific antibodies of the invention to ROR2-expressing, CD3 negative, human tumor cell lines. (A) Binding of bsIgG1-huCD3-H101G-FEAL×chROR2-A-FEAR to human tumor cell lines HeLa (cervical cancer), LCLC103-H (large-cell lung cancer), NCI-H1650 (lung adenocarcinoma), 786-0 (renal cell adenocarcinoma), NCI-H23 (lung adenocarcinoma) and ZR-75-1 (breast ductal carcinoma). Binding was determined by flow cytometry. bsIgG1-huCD3-H101G-FEAL×b12-FEAR, that is able to bind CD3 but not ROR2, was used as negative control antibody. (B) ROR2 expression levels on test tumor cell lines as determined by QIFI analysis; shown are results from separate analyses (n=4-6), median and range. sABC: specific antibody binding capacity.

FIG. 6. In vitro induction of T cell mediated cytotoxicity in co-cultures of ROR2 positive HeLa cells and human healthy donorT cells, at varying effector to target ratios (E:T), in the presence of CD3×ROR2 bispecific antibodies bsIgG1-huCD3-FEAL×chROR2-A-FEAR and bsIgG1-huCD3-H101G-FEAL×chROR2-A-FEAR. bsIgG1-huCD3-FEAL×b12-FEAR, that is able to bind CD3 but not ROR2, was used as negative control antibody. Survival of HeLa cells was used as a read-out for T cell mediated cytotoxicity.

FIG. 7. Induction of cytotoxicity in vitro in various tumor cell lines by CD3×ROR2 bispecific antibodies bsIgG1-huCD3-FEAL×chROR2-A-FEAR and bsIgG1-huCD3-H101G-FEAL×chROR2-A-FEAR in the presence of human healthy donor T cells. bsIgG1-huCD3-FEAL×b12-FEAR, that is able to bind CD3 but not ROR2, was used as negative control antibody. Survival of tumor cells was used as a read-out for T cell mediated cytotoxicity.

FIG. 8. Maximum achieved tumor cell kill of T cell-mediated tumor cell killing in the presence of bsIgG1-huCD3-FEAL×chROR2-A-FEAR or bsIgG1-huCD3-H101G-FEAL×chROR2-A-FEAR for the different tumor cell lines (2-7 donors per cell line). Cell lines are ranked according to ROR2 expression level. The maximum achieved tumor cell kill was determined as the difference between the top and the bottom of the dose response curve, shown as mean and standard deviation (shown either above or below mean, for clarity). The vertical dotted line indicates the lower limit of detection of the QIFI analysis to determine ROR2 expression levels. sABC: specific antibody binding capacity.

FIG. 9. (A) Concentration of cytokine IL-6 in the supernatant of T cell-tumor cell co-cultures with increasing concentrations of antibodies bsIgG1-huCD3-FEAL×chROR2-A-FEAR or bsIgG1-huCD3-H101G-FEAL×chROR2-A-FEAR, using T cells from 2 donors and 786-0 cells as target cells. (B) IFN-gamma, IL-6, IL-8 and IL-10 concentrations associated with T cell mediated cytotoxicity in 50% and 90% tumor cells (IC50 and IC90), as measured in the supernatant of T cell-tumor cell (HeLa or 786-0) co-cultures in the presence of bsIgG1-huCD3-FEAL×chROR2-A-FEAR or bsIgG1-huCD3-H101G-FEAL×chROR2-A-FEAR. Cytokine levels were determined by a multiplex U-plex assay. In (A) dose-response curve is shown. In (B) geometric means and standard deviation (error bars) are shown. Result from two T cell donors per cell line are presented.

FIG. 10. Cytotoxic activity of CD3×ROR2 bispecific antibodies bsIgG1-huCD3-FEAL×ROR2-A-HC4LC3-FEAR and bsIgG1-huCD3-H101G-FEAL×ROR2-A-HC4LC3-FEARin HeLa cells in the presence of cynomolgus monkey PBMCs as a source of T cells in vitro. bsIgG1-huCD3-FEAL×b12-FEAR, that is able to bind CD3 but not ROR2, was used as negative control antibody.

FIG. 11. Induction of T cell activation within the cynomolgus monkey PBMC population in the presence of CD3×ROR2-bispecific antibodies bsIgG1-huCD3-FEAL×ROR2-A-HC4LC3-FEAR and bsIgG1-huCD3-H101G-FEAL×ROR2-A-HC4LC3-FEAR and HeLa cells. T cell activation (% of CD69, CD25 or PD-1 on CD8+ cells) in the presence of the antibodies and HeLa cells, was determined by flow cytometry. bsIgG1-huCD3-FEAL×b12-FEAR that is able to bind CD3 but not ROR2, was used as negative control antibody.

FIG. 12. ROR2 mRNA expression levels in a selection of primary solid tumors. ROR2 mRNA levels were extracted from the Omicsoft TCGA database and visualized using Oncoland software. Indications are ranked according to median of the ROR2 mRNA expression. LIHC=liver hepatocellular carcinoma, renal-all=renal cancer (kidney renal clear cell carcinoma, kidney chromophobe and kidney renal papillary cell carcinoma combined), metast. mel=metastatic skin cutaneous melanoma, prim. melanoma=primary skin cutaneous melanoma, COAD=colon adenocarcinoma, LUAD=lung adenocarcinoma, CESC=cervical squamous cell carcinoma, BLCA=bladder urothelial carcinoma, HNSC=head and neck squamous cell carcinoma, LUSC=lung squamous cell carcinoma, OV=ovarian serous cystadenocarcinoma, BRCA=breast invasive carcinoma, PAAD=pancreatic adenocarcinoma, UCEC=uterine corpus endometrial carcinoma, UCS=uterine carcinosarcoma, SARC=sarcoma.

### DETAILED DESCRIPTION OF THE INVENTION

### Definition

The term “antibody” (Ab) in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g. a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as C1q, the first component in the classical pathway of complement activation. As used herein, unless contradicted by context, the Fc region of an immunoglobulin typically contains at least a CH2 domain and a CH3 domain of an immunoglobulin CH, and may comprise a connecting region, e.g., a hinge region. An Fc-region is typically in dimerized form via, e.g., disulfide bridges connecting the two hinge regions and/or non-covalent interactions between the two CH3 regions. The dimer may be a homodimer (where the two Fc region monomer amino acid sequences are identical) or a heterodimer (where the two Fc region monomer amino acid sequences differ in one or more amino acids). An Fc region-fragment of a full-length antibody can, for example, be generated by digestion of the full-length antibody with papain, as is well-known in the art. An antibody as defined herein may, in addition to an Fc region and an antigen-binding region, further comprise one or both of an immunoglobulin CH1 region and a CL region. An antibody may also be a multi-specific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs (Roche, WO2011069104), strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcAAdp (Regeneron, WO2010151792), Azymetric Scaffold (Zymeworks/Merck, WO2012/058768), mAb-Fv (Xencor, WO2011/028952), Xmab (Xencor), Dual variable domain immunoglobulin (Abbott, DVD-Ig, U.S. Pat. No. 7,612,181); Dual domain double head antibodies (Unilever; Sanofi Aventis, WO20100226923), Di-diabody (ImClone/Eli Lilly), Knobs-into-holes antibody formats (Genentech, WO9850431); DuoBody (Genmab, WO 2011/131746); Bispecific IgG1 and IgG2 (Pfizer/Rinat, WO11143545), DuetMab (MedImmune, US2014/0348839), Electrostatic steering antibody formats (Amgen, EP1870459 and WO 2009089004; Chugai, US201000155133; Oncomed, WO2010129304A2); bispecific IgG1 and IgG2 (Rinat neurosciences Corporation, WO11143545), CrossMAbs (Roche, WO2011117329), LUZ-Y (Genentech), Biclonic (Merus, WO2013157953), Dual Targeting domain antibodies (GSK/Domantis), Two-in-one Antibodies or Dual action Fabs recognizing two targets (Genentech, Novlmmune, Adimab), Cross-linked Mabs (Karmanos Cancer Center), covalently fused mAbs (AIMM), CovX-body (CovX/Pfizer), FynomAbs (Covagen/Janssen ilag), DutaMab (Dutalys/Roche), iMab (MedImmune), IgG-like Bispecific (ImClone/Eli Lilly, Shen, J., et al. J Immunol Methods, 2007. 318(1-2): p. 65-74), TIG-body, DIG-body and PIG-body (Pharmabcine), Dual-affinity retargeting molecules (Fc-DART or Ig-DART, by Macrogenics, WO/2008/157379, WO/2010/080538), BEAT (Glenmark), Zybodies (Zyngenia), approaches with common light chain (Crucell/Merus, U.S. Pat. No. 7,262,028) or common heavy chains (KXBodies by Novlmmune, WO2012023053), as well as fusion proteins comprising a polypeptide sequence fused to an antibody fragment containing an Fc-region like scFv-fusions, like BsAb by ZymoGenetics/BMS, HERCULES by Biogen Idec (US007951918), SCORPIONS by Emergent BioSolutions/Trubion and Zymogenetics/BMS, Ts2Ab (MedImmune/AZ (Dimasi, N., et al. J Mol Biol, 2009. 393(3): p. 672-92), scFv fusion by Genentech/Roche, scFv fusion by Novartis, scFv fusion by Immunomedics, scFv fusion by Changzhou Adam Biotech Inc (CN 102250246), TvAb by Roche (WO 2012025525, WO 2012025530), mAb2 by f-Star (WO2008/003116), and dual scFv-fusion s. It should be understood that the term antibody, unless otherwise specified, includes monoclonal antibodies (such as human monoclonal antibodies), polyclonal antibodies, chimeric antibodies, humanized antibodies, monospecific antibodies (such as bivalent monospecific antibodies), bispecific antibodies, antibodies of any isotype and/or allotype; antibody mixtures (recombinant polyclonals) for instance generated by technologies exploited by Symphogen and Merus (Oligoclonics), multimeric Fc proteins as described in WO2015/158867, and fusion proteins as described in WO2014/031646. While these different antibody fragments and formats are generally included within the meaning of antibody, they collectively and each independently are unique features of the present invention, exhibiting different biological properties and utility.

A “ROR2 antibody” or “anti-ROR2 antibody” as described herein is an antibody which binds specifically to the antigen ROR2, in particular to human ROR2.

A “variant” as used herein refers to a protein or polypeptide sequence which differs in one or more amino acid residues from a parent or reference sequence. A variant may, for example, have a sequence identity of at least 80%, such as 90%, or 95%, or 97%, or 98%, or 99%, to a parent or reference sequence. Also, or alternatively, a variant may differ from the parent or reference sequence by 12 or less, such as 11, 10, 9, 8, 7, 6, 5, 4, 3, 2 or 1 mutation(s) such as substitutions, insertions or deletions of amino acid residues. Accordingly, a “variant antibody” or an “antibody variant”, used interchangeably herein, refers to an antibody that differs in one or more amino acid residues as compared to a parent or reference antibody, e.g., in the antigen-binding region, Fc-region or both. Likewise, a “variant Fc region” or “Fc region variant” refers to an Fc region that differs in one or more amino acid residues as compared to a parent or reference Fc region, optionally differing from the parent or reference Fc region amino acid sequence by 12 or less, such as 11, 10, 9, 8, 7, 6, 5, 4, 3, 2 or 1 mutation(s) such as substitutions, insertions or deletions of amino acid residues. The parent or reference Fc region is typically the Fc region of a human wild-type antibody which, depending on the context, may be a particular isotype. A variant Fc region may, in dimerized form, be a homodimer or heterodimer, e.g., where one of the amino acid sequences of the dimerized Fc region comprises a mutation while the other is identical to a parent or reference wild-type amino acid sequence. Examples of wild-type (typically a parent or reference sequence) IgG CH and variant IgG constant region amino acid sequences, which comprise Fc region amino acid sequences, are set out in Table 1.

The term “immunoglobulin heavy chain” or “heavy chain of an immunoglobulin” as used herein is intended to refer to one of the heavy chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The term “immunoglobulin” as used herein is intended to refer to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized (see for instance Fundamental Immunology Ch. 7 (Paul, W., 2nd ed. Raven Press, N.Y. (1989)). Within the structure of the immunoglobulin, the two heavy chains are inter-connected via disulfide bonds in the so-called “hinge region”. Equally to the heavy chains, each light chain is typically comprised of several regions; a light chain variable region (abbreviated herein as VL) and a light chain constant region. The light chain constant region typically is comprised of one domain, CL. Furthermore, the VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences herein are defined according to IMGT (see Lefranc M P. et al., Nucleic Acids Research, 27, 209-212, 1999] and Brochet X. Nucl. Acids Res. 36, W503-508 (2008)).

When used herein, the terms “half molecule”, “Fab-arm” and “arm” refer to one heavy chain-light chain pair. When a bispecific antibody is described to comprise a half-molecule antibody “derived from” a first antibody, and a half-molecule antibody “derived from” a second antibody, the term “derived from” indicates that the bispecific antibody was generated by recombining, by any known method, said half-molecules from each of said first and second antibodies into the resulting bispecific antibody. In this context, “recombining” is not intended to be limited by any particular method of recombining and thus includes all of the methods for producing bispecific antibodies described herein below, including for example recombining by “half-molecule exchange” also described in the art as “Fab-arm exchange” and the DuoBody® method, as well as recombining at nucleic acid level and/or through co-expression of two half-molecules in the same cells.

The term “antigen-binding region” or “binding region” or antigen-binding domain as used herein, refers to the region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell, bacterium, or virion. The terms “antigen-binding region” and “antigen-binding site” and “antigen-binding domain” may, unless contradicted by the context, be used interchangeably in the context of the present invention.

The terms “antigen” and “target” may, unless contradicted by the context, be used interchangeably in the context of the present invention.

The term “binding” as used herein refers to the binding of an antibody to a predetermined antigen or target, typically with a binding affinity corresponding to a K_{D} of 1E⁻⁶ M or less, e.g. 5E⁻⁷ M or less, 1E⁻⁷ M or less, such as 5E⁻⁸ M or less, such as 1E⁻⁸ M or less, such as 5E⁻⁹ M or less, or such as 1E⁻⁹ M or less, when determined by biolayer interferometry using the antibody as the ligand and the antigen as the analyte and binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its affinity for binding to a non-specific antigen (e.g., BSA, casein) other than the predetermined antigen or a closely-related antigen.

The term “K_{D}” (M), as used herein, refers to the dissociation equilibrium constant of a particular antibody-antigen interaction, and is obtained by dividing k_{d} by kₐ.

The term “k_{d}” (sec⁻¹), as used herein, refers to the dissociation rate constant of a particular antibody-antigen interaction. Said value is also referred to as the k_{off} value or off-rate.

The term “kₐ” (M⁻¹×sec⁻¹), as used herein, refers to the association rate constant of a particular antibody-antigen interaction. Said value is also referred to as the kₒₙ value or on-rate.

The term “ROR2” as used herein, refers to the protein entitled ROR2, also known as receptor tyrosine kinase-like orphan receptor 2, NTRKR2 and neurotrophic tyrosine kinase receptor-related 2, is a single-pass type I transmembrane glycoprotein that belongs to the ROR subfamily of the tyrosine protein kinase family. ROR2 is a tyrosine kinase receptor important in regulating skeletal and neuronal development, cell migration and cell polarity, in part via its proposed role in the non-canonical Wnt5a signaling pathway (Oishi 2003, Genes to cells 8:6450654.) It contains a FZ (frizzled) domain, an Ig (immunoglobulin)-like C2-type domain, and a kringle domain in the extracellular region and a protein kinase domain in the cytoplasmic region (Masiakowski and Carroll 1992, J Biol Chem 267:26181-90). In humans (Homo sapiens), the ROR2 protein has the amino acid sequence shown in SEQ ID NO: 1 (Uniprot accession no. Q01974). In the amino acid sequence shown in SEQ ID NO: 1, amino acid residues 1-31 are a signal peptide, and amino acid residues 32-420 are the mature polypeptide. In cynomolgus monkey (Macaca fascicularis), the ROR2 protein has the amino acid sequence shown in SEQ ID NO: 39 (Uniprot accession no. A0A2K5UT30). In the amino acid sequence shown in SEQ ID NO: 2, amino acid residues 1-34 are a signal peptide, and amino acid residues 35-420 are the mature polypeptide.

The term “CD3” as used herein, refers to the human Cluster of Differentiation 3 protein which is part of the T-cell co-receptor protein complex and is composed of four distinct chains. CD3 is also found in other species, and thus, the term “CD3” is not limited to human CD3 unless contradicted by context. In mammals, the complex contains a CD3γ (gamma) chain (human CD3γ chain UniProtKB/Swiss-Prot No P09693, or cynomolgus monkey CD3γ UniProtKB/Swiss-Prot No Q95L17), a CD3δ (delta) chain (human CD3δ UniProtKB/Swiss-Prot No P04234, or cynomolgus monkey CD3δ UniProtKB/Swiss-Prot No Q95L18), two CD3ε (epsilon) chains (human CD3ε UniProtKB/Swiss-Prot No P07766; amino acid residues 1-22 is a signal peptide and amino acid residues 23-207 is the mature CD3ε polypeptide, which mature protein is identified herein as SEQ ID NO: 21; cynomolgus monkey CD3ε UniProtKB/Swiss-Prot No Q95L15; or rhesus monkey CD3ε UniProtKB/Swiss-Prot No G7NCB9), and a CD3ζ-chain (zeta) chain (human CD3ζ UniProtKB/Swiss-Prot No P20963, cynomolgus monkey CD3ζ UniProtKB/Swiss-Prot No Q09TK0). These chains associate with a molecule known as the T-cell receptor (TCR) and generate an activation signal in T lymphocytes. The TCR and CD3 molecules together comprise the TCR complex.

The term “antibody binding region” refers to a region of the antigen, which comprises the epitope to which the antibody binds. An antibody binding region may be determined by epitope binning using biolayer interferometry, by alanine scan, or by shuffle assays (using antigen constructs in which regions of the antigen are exchanged with that of another species and determining whether the antibody still binds to the antigen or not). The amino acids within the antibody binding region that are involved in the interaction with the antibody may be determined by hydrogen/deuterium exchange mass spectrometry and by crystallography of the antibody bound to its antigen.

The term “epitope” means an antigenic determinant which is specifically bound by an antibody. Epitopes usually consist of surface groupings of molecules such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues which are directly involved in the binding, and other amino acid residues, which are not directly involved in the binding, such as amino acid residues which are effectively blocked or covered by the antibody when it is bound to the antigen (in other words, the amino acid residue is within or closely adjacent to the footprint of the specific antibody).

The terms “monoclonal antibody”, “monoclonal Ab”, “monoclonal antibody composition”, “mAb”, or the like, as used herein refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be produced by a hybridoma which includes a B cell obtained from a transgenic or transchromosomal non-human animal, such as a transgenic mouse, having a genome comprising a human heavy chain transgene and a light chain transgene, fused to an immortalized cell. Monoclonal antibodies may also be produced from recombinantly modified host cells, or systems that use cellular extracts supporting in vitro transcription and/or translation of nucleic acid sequences encoding the antibody.

The term “isotype” as used herein refers to the immunoglobulin class (for instance IgG, IgG1, IgG2, IgG3, IgG4, IgD, IgA, IgE, or IgM) or any allotypes thereof, such as IgG1m(za) and IgG1m(f)) that is encoded by heavy chain constant region genes. Further, each heavy chain isotype can be combined with either a kappa (κ) or lambda (λ) light chain.

The term “full-length antibody” when used herein, refers to an antibody comprising one or two pairs of heavy and light chains, each containing all heavy and light chain constant and variable domains that are normally found in a heavy chain-light chain pair of a wild-type antibody of that isotype. In a full-length variant antibody, the heavy and light chain constant and variable domains may in particular contain amino acid substitutions that improve the functional properties of the antibody when compared to the full-length parent or wild type antibody. A full-length antibody according to the present invention may be produced by a method comprising the steps of (i) cloning the CDR sequences into a suitable vector comprising complete heavy chain sequences and complete light chain sequence, and (ii) expressing the complete heavy and light chain sequences in suitable expression systems. It is within the knowledge of the skilled person to produce a full-length antibody when starting out from either CDR sequences or full variable region sequences. Thus, the skilled person would know how to generate a full-length antibody according to the present invention.

The term “human antibody”, as used herein, is intended to include antibodies having variable and framework regions derived from human germline immunoglobulin sequences and a human immunoglobulin constant domain. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another non-human species, such as a mouse, have been grafted onto human framework sequences.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “Fc region” as used herein, refers to a region comprising, in the direction from the N- to C-terminal end of the antibody, at least a hinge region, a CH2 region and a CH3 region. An Fc region of the antibody may mediate the binding of the immunoglobulin to host tissues or factors, including various cells of the immune system (such as effector cells) and components of the complement system.

The term “hinge region” as used herein refers to the hinge region of an immunoglobulin heavy chain. Thus, for example the hinge region of a human IgG1 antibody corresponds to amino acids 216-230 according to the Eu numbering as set forth in Kabat, E. A. et al., Sequences of proteins of immunological interest. 5th Edition—US Department of Health and Human Services, NIH publication No. 91-3242, pp 662,680,689 (1991). However, the hinge region may also be any of the other subtypes as described herein.

The term “CH1 region” or “CH1 domain” as used herein refers to the CH1 region of an immunoglobulin heavy chain. Thus, for example the CH1 region of a human IgG1 antibody corresponds to amino acids 118-215 according to the Eu numbering as set forth in Kabat (ibid). However, the CH1 region may also be any of the other subtypes as described herein.

The term “CH2 region” or “CH2 domain” as used herein refers to the CH2 region of an immunoglobulin heavy chain. Thus, for example the CH2 region of a human IgG1 antibody corresponds to amino acids 231-340 according to the Eu numbering as set forth in Kabat (ibid). However, the CH2 region may also be any of the other subtypes as described herein.

The term “CH3 region” or “CH3 domain” as used herein refers to the CH3 region of an immunoglobulin heavy chain. Thus, for example the CH3 region of a human IgG1 antibody corresponds to amino acids 341-447 according to the Eu numbering as set forth in Kabat (ibid). However, the CH3 region may also be any of the other subtypes as described herein.

The term “Fc-mediated effector functions,” as used herein, is intended to refer to functions that are a consequence of binding a polypeptide or antibody to its target or antigen on a cell membrane wherein the Fc-mediated effector function is attributable to the Fc region of the polypeptide or antibody. Examples of Fc-mediated effector functions include (i) C1q binding, (ii) complement activation, (iii) complement-dependent cytotoxicity (CDC), (iv) antibody-dependent cell-mediated cytotoxity (ADCC), (v) Fc-gamma receptor (FcgR)-binding, (vi) antibody-dependent, FcγR-mediated antigen crosslinking, (vii) antibody-dependent cellular phagocytosis (ADCP), (viii) complement-dependent cellular cytotoxicity (CDCC), (ix) complement-enhanced cytotoxicity, (x) binding to complement receptor of an opsonized antibody mediated by the antibody, (xi) opsonisation, and (xii) a combination of any of (i) to (xi).

The term “inertness”, “inert” or “non-activating” as used herein, refers to an Fc region which is at least not able to bind any FcγR, induce Fc-mediated cross-linking of FcγRs, or induce FcγR-mediated cross-linking of target antigens via two Fc regions of individual antibodies, or is not able to bind C1q. The inertness of an Fc region of an antibody, may be tested using the antibody in a monospecific or bispecific format. Fc regions having the FEA mutations as described below are examples of inert Fc regions. Thus, in certain embodiments of the invention the Fc region is inert. Therefore, in certain embodiments some or all of the Fc-mediated effector functions are attenuated or completely absent.

The term “full-length” when used in the context of an antibody indicates that the antibody is not a fragment, but contains all of the domains of the particular isotype normally found for that isotype in nature, e.g. the VH, CH1, CH2, CH3, hinge, VL and CL domains for an IgG1 antibody.

The term “monovalent antibody”, in the context of the present invention, refers to an antibody molecule that can interact with a specific epitope on an antigen, with only one antigen binding domain (e.g. one Fab arm). In the context of a bispecific antibody, “monovalent antibody binding” refers to the binding of the bispecific antibody to one specific epitope on an antigen with only one antigen binding domain (e.g. one Fab arm).

The term “monospecific antibody” in the context of the present invention, refers to an antibody that has binding specificity to one epitope only. The antibody may be a monospecific, monovalent antibody (i.e. carrying only one antigen binding region) or a monospecifc, bivalent antibody (i.e. an antibody with two identical antigen binding regions).

The term “bispecific antibody” refers to an antibody having two non-identical antigen binding domains, e.g. two non-identical Fab-arms or two Fab-arms with non-identical CDR regions. In the context of this invention, bispecific antibodies have specificity for at least two different epitopes. Such epitopes may be on the same or different antigens or targets. If the epitopes are on different antigens, such antigens may be on the same cell or different cells, cell types or structures, such as extracellular matrix or vesicles and soluble protein. A bispecific antibody may thus be capable of crosslinking multiple antigens, e.g. two different cells. A particular bispecific antibody of the present invention is capable of binding to ROR2 and CD3 that are typically not expressed on the same cell and is thus capable of crosslinking two different cells that each express one of these targets, such as a tumor cell and a T-cell.

The term “bivalent antibody” refers to an antibody that has two antigen binding regions, which bind to epitopes on one or two targets or antigens or binds to one or two epitopes on the same antigen. Hence, a bivalent antibody may be a monospecific, bivalent antibody or a bispecific, bivalent antibody.

The term “amino acid” and “amino acid residue” may herein be used interchangeably and are not to be understood limiting. Amino acids are organic compounds containing amine (—NH₂) and carboxyl (—COOH) functional groups, along with a side chain (R group) specific to each amino acid. In the context of the present invention, amino acids may be classified based on structure and chemical characteristics. Thus, classes of amino acids may be reflected in one or both of the following tables:

Main Classification Based on Structure and General Chemical Characterization of R Group

| Class | Amino acid |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

Alternative Physical and Functional Classifications of Amino Acid Residues

| Class | Amino acid |
|---|---|
| Hydroxyl group containing | S and T |
| residues | |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | Q, T, K, S, G, P, D, E, and R |

Substitution of one amino acid for another may be classified as a conservative or non-conservative substitution. In the context of the invention, a “conservative substitution” is a substitution of one amino acid with another amino acid having similar structural and/or chemical characteristics, such substitution of one amino acid residue for another amino acid residue of the same class as defined in any of the two tables above: for example, leucine may be substituted with isoleucine as they are both aliphatic, branched hydrophobes. Similarly, aspartic acid may be substituted with glutamic acid since they are both small, negatively charged residues.

In the context of the present invention, a substitution in an antibody is indicated as:
Original amino acid—position—substituted amino acid;

Referring to the well-recognized nomenclature for amino acids, the three-letter code, or one letter code, is used, including the codes “Xaa” or “X” to indicate any amino acid residue. Thus, Xaa or X may typically represent any of the 20 naturally occurring amino acids. The term “naturally occurring” as used herein refers to any one of the following amino acid residues; glycine, alanine, valine, leucine, isoleucine, serine, threonine, lysine, arginine, histidine, aspartic acid, asparagine, glutamic acid, glutamine, proline, tryptophan, phenylalanine, tyrosine, methionine, and cysteine. Accordingly, the notation “K409R” or “Lys409Arg” means, that the antibody comprises a substitution of Lysine with Arginine in amino acid position 409.

Substitution of an amino acid at a given position to any other amino acid is referred to as:
Original amino acid—position; or e.g. “K409”

For a modification where the original amino acid(s) and/or substituted amino acid(s) may comprise more than one, but not all amino acid(s), the more than one amino acid may be separated by “,” or “/”. E.g. the substitution of Lysine with Arginine, Alanine, or Phenylalanine in position 409 is:
“Lys409Arg,Ala,Phe” or “Lys409Arg/Ala/Phe” or “K409R,A,F” or “K409R/A/F” or “K409 to R, A, or F”.

Such designation may be used interchangeably in the context of the invention but have the same meaning and purpose.

Furthermore, the term “a substitution” embraces a substitution into any one or the other nineteen natural amino acids, or into other amino acids, such as non-natural amino acids. For example, a substitution of amino acid K in position 409 includes each of the following substitutions: 409A, 409C, 409D, 409E, 409F, 409G, 409H, 409I, 409L, 409M, 409N, 409Q, 409R, 409S, 409T, 409V, 409W, 409P, and 409Y. This is, by the way, equivalent to the designation 409X, wherein the X designates any amino acid other than the original amino acid. These substitutions may also be designated K409A, K409C, etc. or K409A,C, etc. or K409A/C/etc. The same applies by analogy to each and every position mentioned herein, to specifically include herein any one of such substitutions.

The antibody according to the invention may also comprise a deletion of an amino acid residue. Such deletion may be denoted “del”, and includes, e.g., writing as K409del. Thus, in such embodiments, the Lysine in position 409 has been deleted from the amino acid sequence.

The term “host cell”, as used herein, is intended to refer to a cell into which an expression vector has been introduced. It should be understood that such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO cells, HEK-293 cells, Expi293F cells, PER.C6 cells, NSO cells, and lymphocytic cells, and prokaryotic cells such as E. coli and other eukaryotic hosts such as plant cells and fungi.

The term “transfectoma”, as used herein, includes recombinant eukaryotic host cells expressing the antibody or a target antigen, such as CHO cells, PER.C6 cells, NSO cells, HEK-293 cells, Expi293F cells, plant cells, or fungi, including yeast cells.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment).

The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1). Suitable variants typically exhibit at least about 45%, such as at least about 55%, at least about 65%, at least about 75%, at least about 85%, at least about 90%, at least about 95%, or more (e.g., about 99%) similarity to the parent sequence.

The term “internalized” or “internalization” as used herein, refers to a biological process in which molecules such as the antibody according to the present invention, are engulfed by the cell membrane and drawn into the interior of the cell. Internalization may also be referred to as “endocytosis”.

As used herein, the term “effector cell” refers to an immune cell which is involved in the effector phase of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express Fc receptors (FcRs) or complement receptors and carry out specific immune functions. In some embodiments, an effector cell such as, e.g., a natural killer cell, is capable of inducing ADCC. For example, monocytes, macrophages, neutrophils, dendritic cells and Kupffer cells which express FcRs, are involved in specific killing of target cells and/or presenting antigens to other components of the immune system, or binding to cells that present antigens. In some embodiments the ADCC can be further enhanced by antibody driven classical complement activation resulting in the deposition of activated C3 fragments on the target cell. C3 cleavage products are ligands for complement receptors (CRs), such as CR3, expressed on myeloid cells. The recognition of complement fragments by CRs on effector cells may promote enhanced Fc receptor-mediated ADCC. In some embodiments antibody driven classical complement activation leads to C3 fragments on the target cell. These C3 cleavage products may promote direct complement-dependent cellular cytotoxicity (CDCC). In some embodiments, an effector cell may phagocytose a target antigen, target particle or target cell which may depend on antibody binding and mediated by FcγRs expressed by the effector cells. The expression of a particular FcR or complement receptor on an effector cell may be regulated by humoral factors such as cytokines. For example, expression of FcγRI has been found to be up-regulated by interferon γ (IFN γ) and/or G CSF. This enhanced expression increases the cytotoxic activity of FcγRI-bearing cells against targets. An effector cell can phagocytose a target antigen or phagocytose or lyse a target cell. In some embodiments antibody driven classical complement activation leads to C3 fragments on the target cell. These C3 cleavage products may promote direct phagocytosis by effector cells or indirectly by enhancing antibody mediated phagocytosis.

“Effector T cells” or “Teffs” or “Teff” refers to T lymphocytes that carry out a function of an immune response, such as killing tumor cells and/or activating an antitumor immune-response which can result in clearance of the tumor cells from the body. Examples of Teff phenotypes include CD3+CD4+ and CD3+CD8+. Teffs may secrete, contain or express markers such as IFNγ, granzyme B and ICOS. It is appreciated that Teffs may not be fully restricted to these phenotypes.

As used herein, the term “complement activation” refers to the activation of the classical complement pathway, which is initiated by a large macromolecular complex called C1 binding to antibody-antigen complexes on a surface. C1 is a complex, which consists of 6 recognition proteins C1q and a hetero-tetramer of serine proteases, C1r2C1s2. C1 is the first protein complex in the early events of the classical complement cascade that involves a series of cleavage reactions that starts with the cleavage of C4 into C4a and C4b and C2 into C2a and C2b. C4b is deposited and forms together with C2a an enzymatic active convertase called C3 convertase, which cleaves complement component C3 into C3b and C3a, which forms a C5 convertase This C5 convertase splits C5 in C5a and C5b and the last component is deposited on the membrane and that in turn triggers the late events of complement activation in which terminal complement components C5b, C6, C7, C8 and C9 assemble into the membrane attack complex (MAC). The complement cascade results in the creation of pores in the cell membrane which causes lysis of the cell, also known as complement-dependent cytotoxicity (CDC). Complement activation can be evaluated by using C1q efficacy, CDC kinetics CDC assays (as described in WO2013/004842, WO2014/108198) or by the method Cellular deposition of C3b and C4b described in Beurskens et al., J Immunol Apr. 1, 2012 vol. 188 no. 7, 3532-3541.

The term “treatment” refers to the administration of an effective amount of a therapeutically active antibody variant of the present invention with the purpose of easing, ameliorating, arresting or eradicating (curing) symptoms or disease states.

The term “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody variant are outweighed by the therapeutically beneficial effects.

### SPECIFIC EMBODIMENTS OF THE INVENTION

Antibodies

In a first aspect, the present invention provides an antibody comprising at least one antigen-binding region capable of binding to human ROR2 wherein said antibody comprises a heavy chain variable (VH) region CDR1, CDR2, and CDR3 having the sequences as set forth in SEQ ID NOs: 3, 4, and 5, respectively, and a light chain variable (VL) region CDR1, CDR2, and CDR3 having the sequences as set forth in SEQ ID NO:7, 8 and 9, respectively. Such an antibody may thus be monovalent, bivalent or multivalent for ROR2.

In an embodiment of the invention the antibody comprises two antigen-binding regions capable of binding to human ROR2 wherein said antibody comprises the heavy chain variable (VH) region CDR1, CDR2, and CDR3 having the sequences as set forth in SEQ ID NOs: 3, 4, and 5, respectively, and the light chain variable (VL) region CDR1, CDR2, and CDR3 having the sequences as set forth in SEQ ID NO:7, 8 and 9 respectively. Such an antibody may be a regular bivalent antibody.

In one embodiment of the invention the ROR2 antibody is humanized from an antibody which comprises a VH region having the sequence set forth in SEQ ID NO: 2 and/or a VL region having the sequence set forth in SEQ ID NO: 6 which regions are capable of binding human ROR2. In one embodiment the antibody is humanized from an antibody which is a chimeric antibody comprising rabbit variable heavy chain (VH) forth in SEQ ID NO: 2 and light chain (VL) set forth in SEQ ID NO: 6 and comprising human constant regions such as Ig Kappa light chain and IgG1 allotype G1m (f) heavy chain. One examples of such a chimeric antibody is the is chIgG1-ROR2-A. Hereby a chimeric antibody is provided which has high binding to HeLa cells and which binds human ROR2 and not human ROR1. Such an antibody is a good starting point for providing humanized antibodies with high binding to ROR2 and/or HeLa cells and other ROR2 expressing tumor cells.

While it is within the capacity of the skilled person to humanize an antibody made from a non-human species the humanization of the antibodies according to the invention may be performed as set forth in Example 5 herein. The non-human-species ROR2 antibody may be a rabbit antibody having specificity for human ROR2. Thus, the parent antibody to be humanized may have rabbit VH and VL regions while it may have a human Fc region. The heavy and light chain V region amino acid sequence may be compared against a database of human germline V and J segment sequences in order to identify the heavy and light chain human sequences with the greatest degree of homology for use as human variable domain frameworks. In one embodiment, the germline sequences used as the basis for the humanized designs are IGHV3-23*03, IGHJ2, IGKV1-39*01 and IGKJ4. Accordingly, an antibody of the invention may have CDR regions from a rabbit antibody where the parts of the VH and VL regions outside the CDR regions are humanized. Further, the constant regions of the heavy and light chains are preferably of human origin. The heavy chain constant region or Fc region of the antibody of the invention is preferably a human Fc region of a human immunoglobulin. This may be any human Fc region but may preferably be a human IgG such as IgG1, IgG2, IgG3 or IgG4. In preferred embodiments it is human IgG1. The light chain constant region may in one embodiment be a human kappa light chain. In another embodiment it may be a human lambda light chain.

In embodiments of the invention the antibody comprises a VH region having a sequence selected from the group comprising:
a. a VH region as set forth in SEQ ID NO:10 (HC1);
b. a VH region as set forth in SEQ ID NO:11 (HC2);
c. a VH region as set forth in SEQ ID NO:12 (HC3);
d. a VH region as set forth in SEQ ID NO:13 (HC4);
e. a VH region as set forth in SEQ ID NO:14 (HC5);
f. a VH region as set forth in SEQ ID NO:15 (HC6);
g. a VH region as set forth in SEQ ID NO:16 (HC7) or
h. a VH region having at least 90% sequence identity to any one of the sequences of SEQ ID NOs 10, 11, 12, 13, 14, 15 or 16.

In an embodiment the invention relates to an antibody comprising a VH region having the sequence set forth in SEQ ID NO:10.

In an embodiment the invention relates to an antibody comprising a VH region having the sequence set forth in SEQ ID NO:11.

In an embodiment the invention relates to an antibody comprising a VH region having the sequence set forth in SEQ ID NO:12.

In a preferred embodiment the invention relates to an antibody comprising a VH region having the sequence set forth in SEQ ID NO:13.

In an embodiment the invention relates to an antibody comprising a VH region having the sequence set forth in SEQ ID NO:14.

In an embodiment the invention relates to an antibody comprising a VH region having the sequence set forth in SEQ ID NO:15.

In another embodiment the invention relates to an antibody comprising a VH region having the sequence set forth in SEQ ID NO:16.

In another embodiment the invention relates to an antibody comprising a VH region having at least 90% sequence identity to the sequence set forth in SEQ ID NOs 10.

In another embodiment the invention relates to an antibody comprising a VH region having at least 90% sequence identity to the sequence set forth in SEQ ID NOs 11.

In another embodiment the invention relates to an antibody comprising a VH region having at least 90% sequence identity to the sequence set forth in SEQ ID NOs 12.

In another embodiment the invention relates to an antibody comprising a VH region having at least 90% sequence identity to the sequence set forth in SEQ ID NOs 13.

In another embodiment the invention relates to an antibody comprising a VH region having at least 90% sequence identity to the sequence set forth in SEQ ID NOs 14.

In another embodiment the invention relates to an antibody comprising a VH region having at least 90% sequence identity to the sequence set forth in SEQ ID NOs 15.

In another embodiment the invention relates to an antibody comprising a VH region having at least 90% sequence identity to the sequence set forth in SEQ ID NOs 16.

In another embodiment the invention relates to an antibody comprising a VH region having at least 95% sequence identity to the sequence set forth in SEQ ID NOs 10.

In another embodiment the invention relates to an antibody comprising a VH region having at least 95% sequence identity to the sequence set forth in SEQ ID NOs 11.

In another embodiment the invention relates to an antibody comprising a VH region having at least 95% sequence identity to the sequence set forth in SEQ ID NOs 12.

In another embodiment the invention relates to an antibody comprising a VH region having at least 95% sequence identity to the sequence set forth in SEQ ID NOs 13.

In another embodiment the invention relates to an antibody comprising a VH region having at least 95% sequence identity to the sequence set forth in SEQ ID NOs 14.

In another embodiment the invention relates to an antibody comprising a VH region having at least 95% sequence identity to the sequence set forth in SEQ ID NOs 15.

In another embodiment the invention relates to an antibody comprising a VH region having at least 95% sequence identity to the sequence set forth in SEQ ID NOs 16.

In further embodiments of the invention the antibody comprises a VL region having a sequence selected from the group comprising:
a. a VL region as set forth in SEQ ID NO:17 (LC1);
b. a VL region as set forth in SEQ ID NO:18 (LC2);
c. a VL region as set forth in SEQ ID NO:19 (LC3);
d. a VL region as set forth in SEQ ID NO:20 (LC4); or
e. a VL region having at least 90% sequence identity to any one of the sequences of SEQ ID NOs 17, 18, 19 or 20.

In a further embodiment the invention relates to an antibody comprising a VL region having the sequence set forth in SEQ ID NO:17.

In another embodiment the invention relates to an antibody comprising a VL region having the sequence set forth in SEQ ID NO:18.

In a particular embodiment the invention relates to an antibody comprising a VL region having the sequence set forth in SEQ ID NO:19.

In a further embodiment the invention relates to an antibody comprising a VL region having the sequence set forth in SEQ ID NO:20.

In another embodiment the invention relates to an antibody comprising a VL region having at least 90% sequence identity to the sequence set forth in SEQ ID NOs 17.

In another embodiment the invention relates to an antibody comprising a VL region having at least 90% sequence identity to the sequence set forth in SEQ ID NOs 18.

In another embodiment the invention relates to an antibody comprising a VL region having at least 90% sequence identity to the sequence set forth in SEQ ID NOs 19.

In another embodiment the invention relates to an antibody comprising a VL region having at least 90% sequence identity to the sequence set forth in SEQ ID NOs 20.

In another embodiment the invention relates to an antibody comprising a VL region having at least 95% sequence identity to the sequence set forth in SEQ ID NOs 17.

In another embodiment the invention relates to an antibody comprising a VL region having at least 95% sequence identity to the sequence set forth in SEQ ID NOs 18.

In another embodiment the invention relates to an antibody comprising a VL region having at least 95% sequence identity to the sequence set forth in SEQ ID NOs 19.

In another embodiment the invention relates to an antibody comprising a VL region having at least 95% sequence identity to the sequence set forth in SEQ ID NOs 20.

In a further embodiment the antibody of the invention comprises the VH region having the sequence of SEQ ID NO. 10 and the VL region having the sequence of SEQ ID NO. 17. Such an antibody is named ROR2-A-HC1LC1.

In a further embodiment the antibody of the invention comprises the VH region having the sequence of SEQ ID NO. 10 and the VL region having the sequence of SEQ ID NO. 18. Such an antibody is named ROR2-A-HC1LC2.

In a further embodiment the antibody of the invention comprises the VH region having the sequence of SEQ ID NO. 10 and the VL region having the sequence of SEQ ID NO. 19. Such an antibody is named ROR2-A-HC1LC3.

In a further embodiment the antibody of the invention comprises the VH region having the sequence of SEQ ID NO. 10 and the VL region having the sequence of SEQ ID NO. 20. Such an antibody is named ROR2-A-HC1LC4.

In a further embodiment the antibody of the invention comprises the VH region having the sequence of SEQ ID NO. 11 and the VL region having the sequence of SEQ ID NO. 17. Such an antibody is named ROR2-A-HC2LC1.

In a further embodiment the antibody of the invention comprises the VH region having the sequence of SEQ ID NO. 11 and the VL region having the sequence of SEQ ID NO. 18. Such an antibody is named ROR2-A-HC2LC2.

In a further embodiment the antibody of the invention comprises the VH region having the sequence of SEQ ID NO. 11 and the VL region having the sequence of SEQ ID NO. 19. Such an antibody is named ROR2-A-HC2LC3.

In a further embodiment the antibody of the invention comprises the VH region having the sequence of SEQ ID NO. 11 and the VL region having the sequence of SEQ ID NO. 20. Such an antibody is named ROR2-A-HC2LC4.

In a further embodiment the antibody of the invention comprises the VH region having the sequence of SEQ ID NO. 12 and the VL region having the sequence of SEQ ID NO. 17. Such an antibody is named ROR2-A-HC3LC1.

In a further embodiment the antibody of the invention comprises the VH region having the sequence of SEQ ID NO. 12 and the VL region having the sequence of SEQ ID NO. 18. Such an antibody is named ROR2-A-HC3LC2.

In a further embodiment the antibody of the invention comprises the VH region having the sequence of SEQ ID NO. 12 and the VL region having the sequence of SEQ ID NO. 19. Such an antibody is named ROR2-A-HC3LC3.

In a further embodiment the antibody of the invention comprises the VH region having the sequence of SEQ ID NO. 12 and the VL region having the sequence of SEQ ID NO. 20. Such an antibody is named ROR2-A-HC3LC4.

In a further embodiment the antibody of the invention comprises the VH region having the sequence of SEQ ID NO. 13 and the VL region having the sequence of SEQ ID NO. 17. Such an antibody is named ROR2-A-HC4LC1.

In a further embodiment the antibody of the invention comprises the VH region having the sequence of SEQ ID NO. 13 and the VL region having the sequence of SEQ ID NO. 18. Such an antibody is named ROR2-A-HC4LC2.

In a preferred embodiment the antibody of the invention comprises the VH region having the sequence of SEQ ID NO. 13 and the VL region having the sequence of SEQ ID NO. 19. Such an antibody is named ROR2-A-HC4LC3. Hereby a humanized antibody is provided which has a binding affinity that is very similar to the parent antibody chIgG1-ROR2-A and which is safe to use in humans as it does not raise an immune response when used as treatments in humans.

In a further embodiment the antibody of the invention comprises the VH region having the sequence of SEQ ID NO. 13 and the VL region having the sequence of SEQ ID NO. 20. Such an antibody is named ROR2-A-HC4LC4.

In a further embodiment the antibody of the invention comprises the VH region having the sequence of SEQ ID NO. 14 and the VL region having the sequence of SEQ ID NO. 17. Such an antibody is named ROR2-A-HC5LC1.

In a further embodiment the antibody of the invention comprises the VH region having the sequence of SEQ ID NO. 14 and the VL region having the sequence of SEQ ID NO. 18. Such an antibody is named ROR2-A-HC5LC2.

In a further embodiment the antibody of the invention comprises the VH region having the sequence of SEQ ID NO. 14 and the VL region having the sequence of SEQ ID NO. 19. Such an antibody is named ROR2-A-HC5LC3.

In a further embodiment the antibody of the invention comprises the VH region having the sequence of SEQ ID NO. 14 and the VL region having the sequence of SEQ ID NO. 20. Such an antibody is named ROR2-A-HC5LC4.

In a further embodiment the antibody of the invention comprises the VH region having the sequence of SEQ ID NO. 15 and the VL region having the sequence of SEQ ID NO. 17. Such an antibody is named ROR2-A-HC6LC1.

In a further embodiment the antibody of the invention comprises the VH region having the sequence of SEQ ID NO. 15 and the VL region having the sequence of SEQ ID NO. 18. Such an antibody is named ROR2-A-HC6LC2.

In a further embodiment the antibody of the invention comprises the VH region having the sequence of SEQ ID NO. 15 and the VL region having the sequence of SEQ ID NO. 19. Such an antibody is named ROR2-A-HC6LC3.

In a further embodiment the antibody of the invention comprises the VH region having the sequence of SEQ ID NO. 15 and the VL region having the sequence of SEQ ID NO. 20. Such an antibody is named ROR2-A-HC6LC4.

In a further embodiment the antibody of the invention comprises the VH region having the sequence of SEQ ID NO. 16 and the VL region having the sequence of SEQ ID NO. 17. Such an antibody is named ROR2-A-HC7LC1.

In a further embodiment the antibody of the invention comprises the VH region having the sequence of SEQ ID NO. 16 and the VL region having the sequence of SEQ ID NO. 18. Such an antibody is named ROR2-A-HC7LC2.

In a further embodiment the antibody of the invention comprises the VH region having the sequence of SEQ ID NO. 16 and the VL region having the sequence of SEQ ID NO. 19. Such an antibody is named ROR2-A-HC7LC3.

In a further embodiment the antibody of the invention comprises the VH region having the sequence of SEQ ID NO. 16 and the VL region having the sequence of SEQ ID NO. 20. Such an antibody is named The antibodies according to the invention are characterized by having specificity for or having the ability to bind human (Homo sapiens) ROR2. Hence, ROR2 as referred to herein may in particular be human ROR2, such as the mature polypeptide of SEQ ID NO: 1. In a further embodiment these antibodies do not bind human ROR1.

In further embodiments, the antibodies of the invention are characterized by having specificity for or having the ability to bind to cynomolgus monkey (Macaca fascicularis) ROR2, such as specificity for or the ability to bind to both human and cynomolgus monkey ROR2. Cynomolgus monkey ROR2 may in particular be the mature polypeptide of SEQ ID NO: 39.

In certain embodiments the antibodies of the invention are characterized by having specificity for or having the ability to bind both human (Homo sapiens) ROR2 and cynomolgus monkey (Macaca fascicularis) ROR2. Hereby antibodies are provided which allow for performing non-clinical safety studies in a relevant toxicology species (such as the cynomolgus monkey) using the intended clinical candidate and avoid having to use surrogate antibodies for non-clinical tox studies.

As mentioned above the VH and VL regions of the antibodies of the invention may be humanized such that the ROR2 binding antibodies of the invention in certain embodiments are humanized antibodies and thus are unlikely to raise an immune response in humans when used as a treatment.

It is preferred that the antibodies of the invention have Fc regions that are based on a human type G immunoglobulin. In one embodiment the antibody of the invention has an Fc region which is based on a human IgG1. In another embodiment of the invention the heavy chain constant region is human IgG1. However, it may contain amino acid substitutions as described below. In another embodiment the heavy chain constant region is or is based on human IgG2. In another embodiment the heavy chain constant region is or is based on human IgG3. In another embodiment the heavy chain constant region is or is based on human IgG4. The Fc region may optionally have amino acid modifications to alter the effector functions of the antibody or for other purposes such as for enabling formation of bispecific antibodies of the invention. Such modifications may be substitutions as described further below.

In one embodiment of the invention the antibody light chain constant region is human kappa light chain. In another embodiment of the invention the antibody light chain constant region is human lambda light chain.

In still another embodiment of the invention the antibody is a full-length antibody, such as a full length IgG1 antibody, such as an IgG1 antibody in a regular immunoglobulin format having two binding arms (the Fab region) and an Fc region, which Fc region may be inert as described herein.

In one embodiment the antibody of the invention is a monovalent antibody.

In another embodiment the antibody of the invention is a bivalent antibody.

In yet another embodiment the antibody of the invention is a monospecific antibody.

In another embodiment the antibody of the invention is a bispecific antibody.

As also stated above, the antibody of the invention is capable of binding to human ROR2. In certain embodiments said human ROR2 is the mature protein of SEQ ID NO. 1.

In another embodiment the antibodies provided herein are able to bind to the Kringle domain of human ROR2. The Kringle domain is the amino acids 316-394 of the human ROR2 set forth in SEQ ID NO: 1. Hereby antibodies are provided which bind in a cell membrane-near domain of ROR2.

Also provided herein are antibodies which bind to an epitope or antibody binding region on human ROR2 that involves the amino acid residue at position 322 of human ROR2, wherein the numbering refers to its position in SEQ ID NO: 1.

In an embodiment the antibody of the invention binds human ROR2 extra cellular domain with a binding affinity that corresponds to a KD value of 100 nM or less, such as 50 nM or less, 10 nM or less, 6 nM or less or such as 3 nM or less such as 1.5 nM or less. In another embodiment the antibody binds with a binding affinity corresponding to a KD value which is within the range of 100 nM to 0.1 nM. In another embodiment the antibody binds with a binding affinity corresponding to a KD value which is within the range of 100 nM to 1 nM. In another embodiment the antibody binds with a binding affinity corresponding to a KD value which is within the range of such as 50 nM to 1 nM. In another embodiment the antibody binds with a binding affinity corresponding to a KD value which less than about 2.5 nM or less than about 2.0 nM. In a preferred embodiment the antibody of the invention has a binding affinity to the human ROR2 extra cellular domain which is less than about 1.5 nM, such as about 1.1 nM.

While it is within the capacity of the skilled person to determine the affinity of an antibody for binding to its target, the binding affinity of the antibodies according to the invention for ROR2 may in particular be determined by biolayer interferometry, optionally as set forth in Example 2 or 6 herein.

Thus, the binding affinity may be determined using a biolayer interferometry comprising the steps of:
a. Immobilizing the antibody at an amount of 1 μg/mL for 600 seconds on an anti-human IgG Fc Capture biosensor;
b. Determining association over a time period of 1,500 seconds and dissociation over a time period of 1,500 seconds of his-tagged ROR2 extracellular domain (ROR2-ECD, G&P Biosciences, cat. no. FCL0192) using 2-fold dilution series ranging from 100 nM to 1.56 nM.
c. Referencing the data to a buffer control (0 nM).

Bispecific Antibodies

Examples of bispecific antibody molecules which may be used in the present invention include but are not limited to (i) a single antibody that has two arms comprising different antigen-binding regions, (ii) a single chain antibody that has specificity to two different epitopes, e.g., via two scFvs linked in tandem by an extra peptide linker; (iii) a dual-variable-domain antibody (DVD-Ig™), where each light chain and heavy chain contains two variable domains in tandem through a short peptide linkage Wu et al., Generation and Characterization of a Dual Variable Domain Immunoglobulin (DVD-Ig™) Molecule, In: Antibody Engineering, Springer Berlin Heidelberg (2010); (iv) a chemically-linked bispecific (Fab′)2 fragment; (v) a Tandab®, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so called “dock and lock” molecule (Dock-and-Lock®), based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment, the bispecific antibody of the present invention is a diabody or a cross-body, such as CrossMabs. In a preferred embodiment the bispecific antibody is obtained via a controlled Fab arm exchange (such as described in WO 2011/131746) also known as the DuoBody® technology.

Examples of different classes of bispecific antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv- and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech, WO/2002/020039), the Knobs-into-Holes (Genentech, WO9850431;), CrossMAbs (Roche, WO2011117329) and the electrostatically-matched (Amgen, EP1870459 and WO2009089004; Chugai, US201000155133; Oncomed, WO2010129304), the LUZ-Y (Genentech), DIG-body and PIG-body (Pharmabcine), the Strand Exchange Engineered Domain body (SEEDbody)(EMD Serono, WO2007110205), the Biclonics (Merus), FcAAdp (Regeneron, WO 2010/015792), bispecific IgG1 and IgG2 (Pfizer/Rinat, WO11143545), Azymetric scaffold (Zymeworks/Merck, WO2012058768), mAb-Fv (Xencor, WO2011028952), bivalent bispecific antibodies (Roche WO 2009/080254) and DuoBody® molecules (Genmab A/S, WO 2011/131746). In a preferred embodiment the bispecific antibodies of the invention are DuoBody molecules.

Examples of recombinant IgG-like dual targeting molecules include but are not limited to Dual Targeting (DT)-Ig (GSK/Domantis), Two-in-one Antibody (Genentech), Cross-linked Mabs (Karmanos Cancer Center), mAb2 (F-Star, WO2008003116), Zybodies™ (Zyngenia), approaches with common light chain (Crucell/Merus, U.S. Pat. No. 7,262,028), KXBodies (Novlmmune) and CovX-body (CovX/Pfizer).

Examples of IgG fusion molecules include but are not limited to Dual Variable Domain (DVD)-Ig™ (Abbott, U.S. Pat. No. 7,612,181), Dual domain double head antibodies (Unilever; Sanofi Aventis, WO20100226923), IgG-like Bispecific (ImClone/Eli Lilly), Ts2Ab (MedImmune/AZ) and BsAb (Zymogenetics), HERCULES (Biogen Idec, US007951918), scFv fusion (Novartis), scFv fusion (Changzhou Adam Biotech Inc, CN 102250246) and TvAb (Roche, WO2012025525, WO2012025530).

Examples of Fc fusion molecules include but are not limited to ScFv/Fc Fusions (Academic Institution), SCORPION (Emergent BioSolutions/Trubion, Zymogenetics/BMS), Dual Affinity Retargeting Technology (Fc-DART™) (MacroGenics, WO2008157379, WO2010/080538) and Dual(ScFv)2-Fab (National Research Center for Antibody Medicine—China).

Examples of Fab fusion bispecific antibodies include but are not limited to F(ab)2 (Medarex/AMGEN), Dual-Action or Bis-Fab (Genentech), Dock-and-Lock® (DNL) (ImmunoMedics), Bivalent Bispecific (Biotecnol) and Fab-Fv (UCB-Celltech).

Examples of scFv-, diabody-based and domain antibodies include but are not limited to Bispecific T Cell Engager (BiTE®) (Micromet, Tandem Diabody (Tandab™) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting Nanobodies® (Ablynx), dual targeting heavy chain only domain antibodies.

In a further embodiment the invention provides an antibody comprising a first antigen binding region capable of binding human ROR2 according to the invention as described above and comprising the VH region CDR1, CDR2, and CDR3 of SEQ ID NOs: 3, 4, and 5, respectively, and the VL region CDR1, CDR2, and CDR3 of SEQ ID NO:7, 8 and 9, respectively, and comprising a second antigen binding region capable of binding to a different target. In a particular embodiment the second antigen binding region is capable of binding to human CD3, such as human CD3ε (epsilon), such as human CD3ε (epsilon) as specified in SEQ ID NO: 21. In a preferred embodiment such an antibody of the invention is a bispecific antibody. In another embodiment such an antibody of the invention is a multi-specific antibody.

In a further embodiment the second antigen-binding region which binds to CD3 comprises a VH region comprising the CDR1, CDR2, and CDR3 sequences of SEQ ID NOs.: 23, 24 and 25, respectively, and a VL region comprising the CDR1, CDR2, and CDR3 sequences of SEQ ID NO: 27, GTN and 28, respectively.

In another embodiment the CD3-binding region of the invention comprises a VH and a VL region which are humanized VH and VL regions and which are humanized from the mouse anti human CD3 antibody SP34 having the VH and VL regions of SEQ ID NO: 22 and SEQ ID NO: 26 respectively. As mentioned above it is within the capacity of the skilled person to humanize an antibody. Below are some preferred embodiments of such humanized versions of the mouse SP34 VH and VL regions.

Thus, in an embodiment the antigen binding region that binds to CD3 comprises a VH region having at least 80% amino acid sequence identity to the sequence of SEQ ID NO: 29. In another embodiment the antigen binding region that binds to CD3 comprises a VH region having at least 90% amino acid sequence identity to the sequence of SEQ ID NO: 29. In another embodiment the antigen binding region that binds to CD3 comprises a VH region having at least 95% amino acid sequence identity to the sequence of SEQ ID NO: 29. In another embodiment the antigen binding region that binds to CD3 comprises a VH region having at least 97% amino acid sequence identity to the sequence of SEQ ID NO: 29. In another embodiment the antigen binding region that binds to CD3 comprises a VH region having at least 99% amino acid sequence identity to the sequence of SEQ ID NO: 29. In another embodiment the antigen binding region that binds to CD3 comprises a VH region having the amino acid sequence of SEQ ID NO: 29.

In yet another embodiment the antigen binding region that binds to CD3 comprises a VL region having at least 80% amino acid sequence identity to the sequence of SEQ ID NO: 30. In yet another embodiment the antigen binding region that binds to CD3 comprises a VL region having at least 90% amino acid sequence identity to the sequence of SEQ ID NO: 30. In yet another embodiment the antigen binding region that binds to CD3 comprises a VL region having at least 95% amino acid sequence identity to the sequence of SEQ ID NO: 30. In yet another embodiment the antigen binding region that binds to CD3 comprises a VL region having at least 97% amino acid sequence identity to the sequence of SEQ ID NO: 30. In yet another embodiment the antigen binding region that binds to CD3 comprises a VL region having at least 99% amino acid sequence identity to the sequence of SEQ ID NO: 30. In another embodiment the antigen binding region that binds to CD3 comprises a VL region having the amino acid sequence of SEQ ID NO: 30.

In a preferred embodiment the antigen binding region that binds to CD3 comprises a heavy chain variable region (VH) comprising the sequence of SEQ ID NO: 29, and a light chain variable region (VL) comprising the sequence of SEQ ID NO: 30.

In another embodiment the antibody of the invention comprises a second antigen binding region which has a lower human CD3ε binding affinity than an antibody having an antigen-binding region comprising a VH sequence as set forth in SEQ ID NO: 29, and a VL sequence as set forth in SEQ ID NO: 30. In one embodiment the lower affinity is at least 5-fold lower. In another embodiment the lower affinity is at least 10-fold lower. In another embodiment the lower affinity is at least 20-fold lower. In one embodiment the lower affinity is at least 30-fold lower. In yet another embodiment the lower affinity is at least 40-fold lower. In one embodiment the lower affinity is at least 45-fold lower. In one embodiment the lower affinity is at least 50-fold lower. In one embodiment the lower affinity is at least 54-fold lower. Hereby, CD3 binding regions are provided which have a lower affinity for human CD3 as compared to the antigen-binding region comprising a VH sequence as set forth in SEQ ID NO: 29, and a VL sequence as set forth in SEQ ID NO: 30. When such a CD3 binding region is part of a CD3×ROR2 bispecific antibody the bispecific antibody will have a lower affinity for CD3. This provides bispecific antibodies which may have fewer side effects and are safe to use while still having efficacy in the treatment of disease such as cancer.

In another aspect the invention provides an antibody wherein said antigen binding region that binds to CD3 binds with an equilibrium dissociation constant KD within the range of 200-1000 nM. In one embodiment it binds within the range of 300-1000 nM. In one embodiment it binds within the range of 400-1000 nM. In one embodiment it binds within the range of 500-1000 nM. In one embodiment it binds within the range of 300-900 nM. In one embodiment it binds within the range of 400-900 nM. In one embodiment it binds within the range of 400-700 nM. In one embodiment it binds within the range of 500-900 nM. In one embodiment it binds within the range of 500-800 nM. In one embodiment it binds within the range of 500-700 nM. In one embodiment it binds within the range of 600-1000 nM. In one embodiment it binds within the range of 600-900 nM. In one embodiment it binds within the range of 600-800 nM. In another embodiment it binds within the range of 600-700 nM. These binding affinities for CD3 are considered as lower binding affinity herein.

In another aspect the invention provides an antibody wherein said antigen binding region that binds to CD3 binds with an equilibrium dissociation constant KD within the range of 1-100 nM. In one embodiment it binds within the range of 5-100 nM. In one embodiment it binds within the range of 10-100 nM. In one embodiment it binds within the range of 1-80 nM. In one embodiment it binds within the range of 1-60 nM within the range of 1-40 nM. In one embodiment it binds within the range of 1-20 nM. In one embodiment it binds within the range of 5-80 nM. In one embodiment it binds within the range of 5-60 nM. In one embodiment it binds within the range of 5-40 nM. In one embodiment it binds within the range of 5-20 nM. In one embodiment it binds within the range of 10-80 nM. In one embodiment it binds within the range of 10-60 nM. In one embodiment it binds within the range of 10-40 nM. In one embodiment it binds within the range of 10-20 nM. These binding affinities for CD3 are considered as high binding affinity herein. When such a CD3 binding region is part of a CD3×ROR2 bispecific antibody the bispecific antibody will have a higher affinity for CD3 compared to the lower affinity antibodies described herein. This provides bispecific antibodies which may have higher cytotoxicity against the ROR2 expressing cells and thus having improved efficacy in the treatment of disease such as a ROR2 expressing cancer.

The affinity with which the antibody according to the invention bind to CD3 may be determined by biolayer interferometry, in which the antibody is immobilized on a human IgG Fc Capture biosensor and association and dissociation of the CD3E27-GSKa (SEQ ID NO: 51) to the immobilize antibody is determined. Further, the affinity with which the antibody according to the invention bind to CD3 may be determined by biolayer interferometry as provided in Example 9 herein.

Antibodies binding CD3, in particular human CD3, with reduced affinity are provided in WO 2017/009442, and it is to be understood that any of these antibodies may serve as the basis for generating antibodies according to the present invention which in addition to the ability to bind ROR2 also have the ability to bind CD3 with reduced affinity.

In a particular embodiment the antigen binding region of the antibody that binds to CD3 comprises a heavy chain variable (VH) region comprising a CDR1 sequence, a CDR2 sequence and a CDR3 sequence of the heavy chain variable region of SEQ ID NO: 29 but comprises an amino acid substitution in one of the CDR sequences, the substitution being at a position selected from the group consisting of: T31, N57, H101, G105, S110 and Y114, the positions being numbered according to the sequence of SEQ ID NO: 29; and comprises the wild type light chain variable (VL) region comprises the CDR1, CDR2 and CDR3 sequences set forth in SEQ ID NO: 27, GTN and SEQ ID NO: 28, respectively. The CDR sequences herein are defined according to IMGT.

In one embodiment the substitution in the CD3 binding region of the antibody is at position T31. In another embodiment the substitution is at position. In another embodiment the substitution is at position N57. In another embodiment the substitution is at position H101. In another embodiment the substitution is at position G105. In another embodiment the substitution is at position S110. In another embodiment the substitution is at position Y114.

In another embodiment of the antibody the CDR1, CDR2 and CDR3 of the heavy chain variable region of the antigen binding region that binds to CD3 comprises at most 1, 2, 3, 4 or 5 amino acid substitutions in total, when compared to the CDR1, CDR2 and CDR3 of the sequence set forth in SEQ ID NO: 29. In one embodiment it only has one substation in one of the CDR regions. In another embodiment it has two substitutions in total in one of the CDR regions or in two different regions. In another embodiment it has three substitutions in total in one or more of the CDR regions. In another embodiment it has four substitutions in total in one or more of the CDR regions. In another embodiment it has three substitutions in total in one or more of the CDR regions. In another embodiment it has five substitutions in total in one or more of the CDR regions.

In a further embodiment the antigen binding region of the antibody that binds to CD3 comprises an amino acid substitution in the VH region of SEQ ID NO: 29 selected from the group consisting of: T31M, T31P, N57E, H101G, H101N, G105P, S110A, S110G, Y114M, Y114R, Y114V wherein the numbering refers to the position of SEQ ID NO: 29. In one embodiment the substitution is T31M. In another embodiment the substitution is T31P. In another embodiment the substitution is N57E. In another embodiment the substitution is H101G. In another embodiment the substitution is H101N. In another embodiment the substitution is G105P. In another embodiment the substitution is S110A. In another embodiment the substitution is S110G. In another embodiment the substitution is Y114M. In another embodiment the substitution is Y114M. In another embodiment the substitution is Y114R. In another embodiment the substitution is Y114V.

In one embodiment the invention provides an antibody wherein the antigen-binding region which is capable of binding to CD3 comprises a heavy chain variable region (VH) comprising CDR1, CDR2, and CDR3 having the sequences as set forth in SEQ ID NOs: 23, 24, and 31, respectively, and a light chain variable region (VL) comprising CDR1, CDR2, and CDR3 having the sequences as set forth in SEQ ID NO: 27, the sequence GTN, and the sequence as set forth in SEQ ID NO: 28, respectively. This bispecific antibody has a lower affinity for CD3 as described above, when compared to an identical antibody except for having a VH-CDR3 region of SEQ ID NO: 25. Hereby a bispecific CD3×ROR2 antibody is provided which has a lower affinity for CD3. Such an antibody is useful in the treatment of diseases such as ROR2 expressing tumors and may have fewer side effects such as e.g. milder cytokine release syndrome compared to a version of the bispecific antibody with a higher affinity for CD3. It may in certain situations be an advantage that such a bispecific antibody of the invention can be dosed at higher concentrations.

In one embodiment the invention provides an antibody wherein the antigen-binding region capable of binding to CD3 comprises a heavy chain variable region (VH) comprising the sequence set forth in SEQ ID NO: 32 and a light chain variable region (VL) comprising the sequence set forth in SEQ ID NO: 30. Hereby a lower affinity CD3 binding arm is provided for the bispecific antibody of the invention.

In a main embodiment the invention provides a bispecific antibody comprising a first antigen binding region capable of binding to human ROR2 and a second binding region capable of binding to human CD3, wherein said first antigen binding region comprises:
a heavy chain variable (VH) region CDR1, CDR2, and CDR3 having the sequences as set forth in SEQ ID NOs:3, 4, and 5, respectively, and a light chain variable (VL) region CDR1, CDR2, and CDR3 having the sequences as set forth in SEQ ID NO:7, 8 and 9, respectively;
   and said second antigen binding region comprises:
a heavy chain variable (VH) region CDR1, CDR2, and CDR3 having the sequences as set forth in NOs.: 23, 24 and 25, respectively; and a light chain variable region (VL) comprising the CDR1, CDR2, and CDR3 sequences of SEQ ID NO: 27, GTN and 28, respectively.

Hereby a CD3×ROR2 bispecific antibody is provided which has high affinity for CD3. Such an antibody is useful in the treatment of diseases such as ROR2 expressing tumors. The higher affinity version of the bispecific antibody may have the advantage that it can be dosed at lower concentrations and/or less frequently. It may also be more potent and thus more cytotoxic compared to the lower affinity CD3×ROR2 bispecific antibody.

In another embodiment the invention provides a bispecific antibody comprising a first antigen binding region capable of binding to human ROR2 and a second binding region capable of binding to human CD3, wherein said first antigen binding region comprises:
a heavy chain variable (VH) region CDR1, CDR2, and CDR3 having the sequences as set forth in SEQ ID NOs: 3, 4, and 5, respectively, and a light chain variable (VL) region CDR1, CDR2, and CDR3 having the sequences as set forth in SEQ ID NO: 7, 8 and 9, respectively;
   and said second antigen binding region comprises:
a heavy chain variable (VH) region comprising CDR1, CDR2, and CDR3 having the sequences as set forth in SEQ ID NOs: 23, 24, and 31, respectively, and a light chain variable region (VL) comprising CDR1, CDR2, and CDR3 having the sequences as set forth in SEQ ID NO: 27, the sequence GTN, and the sequence as set forth in SEQ ID NO: 28, respectively.

Hereby a CD3×ROR2 bispecific antibody is provided which has lower affinity for CD3 compared to the variant having the VH CDR3 region of SEQ ID NO 25. Such an antibody is likewise useful in the treatment of diseases such as ROR2 expressing tumors as also mentioned above. In certain situations, such a bispecific antibody may be better tolerated and safer to use in humans.

The invention further provides a bispecific antibody wherein said antibody comprises a first antigen binding region capable of binding to human ROR2 and a second antigen binding region capable of binding to human CD3, wherein said first antigen binding region comprises a VH region comprising the sequence as set forth in SEQ ID NO: 13, and a VL region comprising the sequence as set forth in SEQ ID NO: 19, and said second antigen binding region comprises a VH region comprising the sequence as set forth in SEQ ID NO: 29, and a VL region comprising the sequence as set forth in SEQ ID NO: 30.

The invention further provides a bispecific antibody wherein said antibody comprises a first antigen binding region capable of binding to human ROR2 and a second antigen binding region capable of binding to human CD3, wherein said first antigen binding region comprises a VH region comprising the sequence as set forth in SEQ ID NO: 13, and a VL region comprising the sequence as set forth in SEQ ID NO: 19, and said second antigen binding region comprises a VH region comprising the sequence as set forth in SEQ ID NO: 32, and a VL region comprising the sequence as set forth in SEQ ID NO: 30.

In one embodiment of the invention the antigen-binding region(s) capable of binding to ROR2 is/are humanized. In one embodiment the second antigen-binding region capable of binding to CD3, if present, is humanized.

In some embodiments, the antibody according to the present invention comprises, in addition to the antigen-binding regions, an Fc region consisting of the Fc sequences of the two heavy chains. The first and second Fc sequence may each be of any isotype, including any human isotype, such as an IgG1, IgG2, IgG3, IgG4, IgE, IgD, IgM, or IgA isotype or a mixed isotype. Preferably, the Fc region is a human IgG1, IgG2, IgG3, IgG4 isotype or a mixed isotype, such as a human IgG1 isotype.

In particular embodiments, the antibody according to the invention comprises a first and a second heavy chain, such as a first and second heavy chain each comprising at least a hinge region, a CH2 and CH3 region. Stable, heterodimeric antibodies can be obtained at high yield for instance by so-called Fab-arm exchange as provided in WO 2011/131746, on the basis of two homodimeric starting proteins containing only a few, asymmetrical mutations in the CH3 regions. Hence, in some embodiments of the invention the bispecific antibody comprises a first and a second heavy chain constant region, each of said first and second heavy chain constant regions comprises at least a hinge region, a CH2 and CH3 region, wherein in said first heavy chain constant region at least one of the amino acids in the positions corresponding to positions selected from the group consisting of T366, L368, K370, D399, F405, Y407 and K409 in a human IgG1 heavy chain has been substituted, and in said second heavy chain constant region at least one of the amino acids in the positions corresponding to a position selected from the group consisting of T366, L368, K370, D399, F405, Y407, and K409 in a human IgG1 heavy chain has been substituted, wherein said substitutions of said first and said second heavy chains are not in the same positions, and wherein the amino acid positions in the constant regions are numbered according to Eu numbering.

In a preferred embodiment the constant region of the heavy chains of the ROR2 binding antibody of the invention comprises the amino acid R in the position corresponding to K409 in a human IgG1 heavy chain. It is preferred that the heavy chain constant regions are IgG1, but they can also be other isotypes such as e.g. IgG4. Accordingly, the ROR2 antibody preferably has an arginine at position 409 of its heavy chains. In a preferred embodiment the CD3 binding arm has a leucine in position 405 of its heavy chains when using the Eu numbering system.

Thus, in one embodiment the invention provides a bispecific antibody wherein the first heavy chain constant region has the amino acid arginine (R) at position 409 and the second heavy chain constant region has the amino acid leucine (L) at position 405 wherein the numbering is according to the Eu numbering system.

In another embodiment the invention provides a bispecific antibody wherein the first heavy chain constant region has the amino acid arginine (R) at position 409 and the amino acid phenylalanine (F) at position 405 and the second heavy chain constant region has the amino acid lysine (K) at position 409 and the amino acid leucine (L) at position 405.

Further, the antibody according to the invention is preferably an antibody that, when assessed by flow cytometry or ELISA, does not bind FcγRs, and consequently does not induce FcγR-mediated effector functions including CD3-antibody dependent, FcγR-mediated CD3-crosslinking in absence of target (ROR2)-specific tumor cells. Further, the antibody according to the invention is preferably an antibody that, when assessed by flow cytometry or ELISA, does not bind C1q and consequently is unable to induce complement-dependent effector functions. In preferred embodiments, the antibody of the invention does not bind FcγR and does not bind C1q.

In another embodiment the invention provides an antibody which comprises a first and a second heavy chain and wherein the first and second heavy chains are modified so that the antibody induces Fc-mediated effector function to a lesser extent relative to an identical non-modified antibody.

Antibodies according to the present invention may comprise modifications in the Fc region to render the antibody an inert, or non-activating, antibody. Hence, in the antibodies disclosed herein, one or both heavy chains may be modified so that the antibody induces Fc-mediated effector function to a lesser extent relative to an antibody which is identical, except for comprising non-modified first and second heavy chains. The Fc-mediated effector function may be measured by determining Fc-mediated CD69 expression on T cells (i.e. CD69 expression as a result of CD3 antibody-mediated, Fcγ receptor-dependent CD3 crosslinking), by determining binding to Fcγ receptors, by determining binding to C1q, or by determining induction of Fc-mediated cross-linking of FcγRs. In particular, the heavy chain constant sequences may be modified so that the Fc-mediated CD69 expression is reduced by at least 50%, at least 60%, at least 70%, at least 80%, at least 90%, at least 99% or 100% when compared to a wild-type (unmodified) antibody, wherein said Fc-mediated CD69 expression is determined in a PBMC-based functional assay, e.g. as described in Example 3 of WO2015001085. Modifications of the heavy and light chain constant sequences may also result in reduced binding of C1q to said antibody. As compared to an unmodified antibody the reduction may be by at least 70%, at least 80%, at least 90%, at least 95%, at least 97%, or 100% and the C1q binding may be determined by ELISA. Further, the Fc region which may be modified so that said antibody mediates reduced Fc-mediated T-cell proliferation compared to an unmodified antibody by at least 50%, at least 60%, at least 70%, at least 80%, at least 90%, at least 99% or 100%, wherein said T-cell proliferation is measured in a PBMC-based functional assay.

Examples of amino acid positions that may be modified, e.g. in an IgG1 isotype antibody, include positions L234 and L235. Hence, in one embodiment the invention provides an antibody which comprises a first and a second heavy chain, wherein in both the first and the second heavy chain constant region, the amino acid residues at the positions corresponding to positions L234 and L235 in a human IgG1 heavy chain according to Eu numbering are F and E, respectively.

In addition, a D265A amino acid substitution can decrease binding to all Fcγ receptors and prevent ADCC (Shields et al., 2001, J. Biol. Chem. (276):6591-604). Therefore, in another embodiment the antibody comprises a first and a second heavy chain, wherein in both the first and the second heavy chain constant region, the amino acid residue at the position corresponding to position D265 in a human IgG1 heavy chain according to Eu numbering is A.

In another embodiment the antibody comprises a first and a second heavy chain, and wherein in both the first and the second heavy chain constant regions, the amino acid residue at the position corresponding to positions L234, L235 and D265 in a human IgG1 heavy chain according to Eu numbering are F, E and A respectively. Hereby an antibody is provided which has an inert Fc region.

In another embodiment the invention provides an antibody which comprises a first and a second heavy chain, and wherein in both the first and the second heavy chain constant regions, the amino acid residue at the position corresponding to positions L234, L235 and D265 in a human IgG1 heavy chain according to Eu numbering are F, E and A respectively and wherein the first heavy chain constant region further comprises an R at position 409 and the second heavy chain constant region further comprises an L at position 405. Hereby an antibody is provided which antibody induces Fc-mediated effector function to a lesser extent relative to an identical non-modified antibody. The amino acids at position 409 and 405 are useful in the process of producing the bispecific antibody using the DuoBody® method also known as the controlled Fab arm exchange method, see Example 10. In the present application antibodies, which have the combination of three amino acid substitutions L234F, L235E and D265A and in addition the K409R or the F405L mutation disclosed herein above are termed with the suffix “FEAR” or “FEAL”, respectively.

The amino acid sequence of the wild type IgG1 heavy chain constant region is identified herein as SEQ ID NO: 33. Consistent with the embodiments disclosed above, the antibody of the invention may comprise an IgG1 heavy chain constant region carrying the F405L substitution and having the amino acid sequence set forth in SEQ ID NO: 38 and/or an IgG1 heavy chain constant region carrying the K409R substitution and having the amino acid sequence set forth in SEQ ID NO: 49.

The amino acid sequence of an IgG1 heavy chain constant region carrying the L234F, L235E and D265A substitutions is identified herein as SEQ ID NO: 50. The amino acid sequence of an IgG1 heavy chain constant region carrying the L234F, L235E, D265A and F405L substitutions is identified herein as SEQ ID NO: 35. The amino acid sequence of an IgG1 heavy chain constant region carrying the L234F, L235E, D265A and K409R substitutions is identified herein as SEQ ID NO: 34.

Hence, the invention provides an antibody which comprises a first and a second heavy chain constant region having the sequences as set forth in SEQ ID Nos 34 and 35, respectively, or a first and a second heavy chain constant region having the sequences as set forth in SEQ ID Nos 35 and 34, respectfully.

In another embodiment the antibody is a bispecific antibody capable of binding to human ROR2 and human CD3 epsilon wherein
a. the first binding arm binding to ROR2 comprises:
   i. The VH region having the amino acid sequence of SEQ ID NO: 13
   ii. The VL region having the amino acid sequence of SEQ ID NO: 19
   iii. A heavy chain constant region having the amino acid sequence of SEQ ID NO: 34 (FEAR) and
   iv. A human kappa light chain constant region; and
b. the second binding arm binding to CD3 epsilon comprises:
   i. The VH region having the amino acid sequence of SEQ ID NO: 29
   ii. The VL region having the amino acid sequence of SEQ ID NO: 30
   iii. A heavy chain constant region having the amino acid sequence of SEQ ID NO: 35 (FEAL) and
   iv. A human lambda light chain constant region.

In another embodiment the antibody is a bispecific antibody capable of binding to human ROR2 and human CD3 epsilon wherein
a. the first binding arm binding to ROR2 comprises:
   i. The VH region having the amino acid sequence of SEQ ID NO: 13
   ii. The VL region having the amino acid sequence of SEQ ID NO: 19
   iii. A heavy chain constant region having the amino acid sequence of SEQ ID NO: 34 (FEAR) and
   iv. A human kappa light chain constant region; and
b. the second binding arm binding to CD3 epsilon comprises:
   i. The VH region having the amino acid sequence of SEQ ID NO: 32
   ii. The VL region having the amino acid sequence of SEQ ID NO: 30
   iii. A heavy chain constant region having the amino acid sequence of SEQ ID NO: 35 (FEAL) and
   iv. A human lambda light chain constant region.

In one embodiment the antibody according to the invention comprises a lambda (λ) light chain. In another embodiment the antibody according to the invention comprises a kappa light chain. The human kappa light chain preferably has the sequence set forth in SEQ ID NO: 36. The human lambda light chain preferably has the sequence set forth in SEQ ID NO: 37.

In particular embodiments, the antibody comprises a lambda (λ) light chain and a kappa (κ) light chain; e.g. an antibody with a heavy chain and a lambda light chain which comprise the binding region capable of binding to CD3, and a heavy chain and a kappa light chain which comprise the binding region capable of binding to ROR2.

The capacity of CD3×ROR2 bispecific antibodies to induce activation of T cells in vitro in the presence of ROR2 expressing tumor cells such as HeLa cells may be determined in a procedure comprising the steps of:
i) Providing T cells isolated from healthy human donor buffy coats,
ii) Providing a set of samples, wherein each sample comprises said T cells and ROR2 expressing tumor cells and wherein the ratio of T cells: tumor cells in said samples is 8:1,
iii) adding the antibody to the set of samples at concentrations ranging from 0.0005 ng/mL to 10,000 ng/mL (such as 5-fold dilutions) and incubating the samples for 72 hours at 37° C.,
iv) collecting from each sample 150 μL supernatant containing T cells and staining the T cells with fluorescent-labeled antibodies against T-cell markers, such as CD3, CD4, CD8, and with fluorescent-labeled antibodies against T-cell activation markers, such as CD69, CD25 and CD279/PD1-B, by incubation with said antibodies for 30 minutes at 4° C.; and
v) analyzing the samples by flow cytometry.

The ability of CD3×ROR2 bispecific antibodies to induce cytotoxicity of ROR2 expressing tumor cells may be determined in a procedure comprising the steps of
i) Providing T cells isolated from healthy human donor buffy coats,
ii) Providing a set of test samples and control samples, wherein each sample comprises said T-cells and ROR2 tumor cells which have been allowed to adhere to the bottom of a 96-well tissue culture plate and wherein the ratio of T-cells: tumor cells in said samples is 8:1,
iii) adding the antibody to the set of test samples at concentrations ranging from 0.0005 ng/mL to 10,000 ng/mL (such as 5-fold dilutions), while the control samples remain untreated or are incubated with 16 μg/mL phenylarsine oxide (PAO), and incubating all samples for 72 hours at 37° C.,
iv) Incubating the adherent cells in 10% (w/w) alamarBlue solution in RPMI-1640 medium supplemented with 10% (w/w) donor bovine serum with iron and penecilin/streptomycin at 37° C. for 4 hours,
v) Measuring the absorbance of the cells; setting the absorbance of the cells incubated with PAO as 0% viability and the untreated cells as 100% viability and calculating the percentage viable cells as

$$\times 100 \% viable cells = ( \frac{[ a b s o r bance sample - absorbance PAO treated cells ]}{[ a b s o r bance untreated cells - absorbance PAO treated cells ]} )$$

In particular embodiments the antibody according to the invention:
a. Is capable of binding to ROR2 expressing human tumor cells such as HeLa, LCLC103-H, NCI-H1650, 786-0, NCI-H23 or ZR-75-1 cells such as described in Example 3, 7 and 12 herein
b. is capable of mediating concentration-dependent cytotoxicity of HeLa cells, when using purified PBMCs or T cells as effector cells e.g. when assayed as described in Example 13 or 14 herein,
c. is capable of mediating concentration-dependent cytotoxicity of 786-0, LCLC-103H, NCI-H23, NCH-H1650 or ZR-75-1 cells, when using purified PBMCs or T cells as effector cells e.g. when assayed as described in Example 14 herein,
d. is capable of activating T cells in vitro in the presence of HeLa tumor cells; e.g. when assayed as described in Example 16 herein, and/or
e. is capable of inducing T cell cytokine production when using tumor cells such as HeLa and 786-0 cells as target cells e.g. when assayed as described in Example 15 herein.

Nucleic Acid Constructs

A further aspect of the invention provides nucleic acid construct comprising
a. a nucleic acid sequence encoding a heavy chain sequence of an antibody comprising an antigen-binding region capable of binding to ROR2 as defined herein before, and/or
b. a nucleic acid sequence encoding a light chain sequence of an antibody comprising an antigen-binding region capable of binding to ROR2 as defined herein before.

The nucleic acid construct may further comprise:
a. a nucleic acid sequence encoding a heavy chain sequence of an antibody comprising an antigen-binding region capable of binding to CD3 as defined herein before; and/or
b. a nucleic acid sequence encoding a light chain sequence of an antibody comprising an antigen-binding region capable of binding to CD3 as defined herein before.

A further aspect of the invention provides one or more nucleic acids comprising:
a. a nucleic acid sequence encoding a heavy chain sequence of an antibody comprising an antigen-binding region capable of binding to ROR2 as defined in SEQ ID NO: 13,
b. a nucleic acid sequence encoding the corresponding light chain sequence of an antibody comprising said antigen-binding region capable of binding to ROR2 as defined in SEQ ID NO: 19.

In a further aspect of the invention said nucleic acid is RNA or DNA.

The nucleic acids of the invention may be for use in expression in mammalian cells.

Expression Vectors

Another aspect of the invention provides an expression vector comprising nucleic acid sequences encoding heavy and/or light chain sequences of an antibody according to the invention. In particular, the expression vector may comprise:
a) a nucleic acid sequence encoding a heavy chain sequence of an antibody comprising an antigen-binding region capable of binding to ROR2 as defined herein before, and/or
b) a nucleic acid sequence encoding a light chain sequence of an antibody comprising an antigen-binding region capable of binding to ROR2 as defined herein before.

The expression vector may further comprise:
a) a nucleic acid sequence encoding a heavy chain sequence of an antibody comprising an antigen-binding region capable of binding to CD3 as defined herein before; and/or
b) a nucleic acid sequence encoding a light chain sequence of an antibody comprising an antigen-binding region capable of binding to CD3 as defined herein before.

In a further embodiment, the expression vector further comprises a nucleic acid sequence encoding the constant region of a light chain, a heavy chain or both light and heavy chains of an antibody, e.g. a human IgG1,κ monoclonal antibody.

An expression vector in the context of the present invention may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, an anti-ROR2 antibody-encoding nucleic acid is comprised in a naked DNA or RNA vector, including, for example, a linear expression element (as described in for instance Sykes and Johnston, Nat Biotech 17, 355 59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793 800 (2001)), or as a precipitated nucleic acid vector construct, such as a CaP04-precipitated construct (as described in for instance WO 00/46147, Benvenisty and Reshef, PNAS USA 83, 9551 55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the anti-ROR2 antibody in a bacterial cell. Examples of such vectors include expression vectors such as BlueScript (Stratagene), pIN vectors Van Heeke & Schuster, J Biol Chem 264, 5503 5509 (1989), pET vectors (Novagen, Madison WI) and the like).

An expression vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516 544 (1987)).

A nucleic acid construct and/or vector may also comprise a nucleic acid sequence encoding a secretion/localization sequence, which can target a polypeptide, such as a nascent polypeptide chain, to the periplasmic space or into cell culture media. Such sequences are known in the art, and include secretion leader or signal peptides, organelle targeting sequences (e. g., nuclear localization sequences, ER retention signals, mitochondrial transit sequences, chloroplast transit sequences), membrane localization/anchor sequences (e. g., stop transfer sequences, GPI anchor sequences), and the like.

In an expression vector of the invention, anti-ROR2 antibody-encoding nucleic acids may comprise or be associated with any suitable promoter, enhancer, and other expression-facilitating elements. Examples of such elements include strong expression promoters (e.g., human CMV IE promoter/enhancer as well as RSV, SV40, SL3 3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE (the skilled artisan will recognize that such terms are actually descriptors of a degree of gene expression under certain conditions).

In one embodiment, the anti-ROR2-antibody-encoding expression vector may be positioned in and/or delivered to a host cell or host animal via a viral vector.

Cells and Host Cells

In a further aspect, the invention provides a cell comprising a nucleic acid construct as defined herein above, or an expression vector as defined herein above. It is to be understood that the cell may have been obtained by transfecting a host cell with said nucleic acid construct or expression vector, such as a recombinant host cell.

The host cell may be of human origin, such as a human embryonic kidney (HEK) cell, such as a HEK/Expi cell. Alternatively, it may be of rodent origin, such as a Chinese hamster ovary cell, such as a CHO/N50 cell. Further, the host cell may be of bacterial origin.

The cell may comprise a nucleic acid sequence encoding an antibody of the invention or parts thereof stably integrated into the cellular genome. Alternatively, the cell may comprise a non-integrated nucleic acid, such as a plasmid, cosmid, phagemid, or linear expression element, which comprises a sequence coding for expression of an anti-ROR2 antibody of the invention or a part thereof. In particular, the host cell may comprise a non-integrated nucleic acid, such as a plasmid, cosmid, phagemid, or linear expression element, which comprises a sequence coding for expression of an anti-ROR2 antibody or a part thereof.

Compositions

A still further aspect of the invention provides a composition comprising an antibody; e.g. a bispecific antibody as defined in the above. The composition may be a pharmaceutical composition comprising the antibody or the bispecific antibody and a pharmaceutically acceptable carrier.

The pharmaceutical compositions may be formulated with the carrier, excipient and/or diluent as well as any other components suitable for pharmaceutical compositions, including known adjuvants, in accordance with conventional techniques such as those disclosed in Remington: The Science and Practice of Pharmacy, 19th Edition, Gennaro, Ed., Mack Publishing Co., Easton, P A, 1995. The pharmaceutically acceptable carriers or diluents as well as any known adjuvants and excipients should be suitable for the antibody or antibody conjugate of the present invention and the chosen mode of administration. Suitability for carriers and other components of pharmaceutical compositions is determined based on the lack of significant negative impact on the desired biological properties of the chosen compound or pharmaceutical composition of the present invention (e.g., less than a substantial impact [10% or less relative inhibition, 5% or less relative inhibition, etc.] upon antigen binding).

A pharmaceutical composition of the present invention may include diluents, fillers, salts, buffers, detergents (e. g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Pharmaceutically acceptable carriers include any and all suitable solvents, dispersion media, coatings, antibacterial and antifungal agents, isotonicity agents, antioxidants and absorption-delaying agents, and the like that are physiologically compatible with a compound of the present invention.

Examples of suitable aqueous and non-aqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the present invention is contemplated.

Pharmaceutical compositions of the present invention may also comprise pharmaceutically acceptable antioxidants for instance (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Pharmaceutical compositions of the present invention may also comprise isotonicity agents, such as sugars, polyalcohols, such as mannitol, sorbitol, glycerol or sodium chloride in the compositions.

The pharmaceutical compositions of the present invention may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The compounds of the present invention may be prepared with carriers that will protect the compound against rapid release, such as a controlled release formulation, including implants, transdermal patches, and micro-encapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, poly-ortho esters, and polylactic acid alone or with a wax, or other materials well known in the art. Methods for the preparation of such formulations are generally known to those skilled in the art, see e.g. Sustained and Controlled Release Drug Delivery Systems, J. R. Robinson, ed., Marcel Dekker, Inc., New York, 1978.

In one embodiment, the compounds of the present invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except in so far as any conventional media or agent is incompatible with the active compound, use thereof in the pharmaceutical compositions of the present invention is contemplated. Other active or therapeutic compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or a non-aqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, by the use of a coating such as lecithin, by the maintenance of the required particle size in the case of dispersion and by the use of surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, examples of methods of preparation are vacuum-drying and freeze-drying (lyophilization) that yield a powder of the active ingredient plus any additional desired ingredient from a previously sterile-filtered solution thereof.

The pharmaceutical composition of the present invention may contain one antibody or bispecific antibody of the present invention, a combination of an antibody and a bispecific antibody according to the invention with another therapeutic compound, or a combination of compounds of the present invention.

The pharmaceutical composition may be administered by any suitable route and mode. Suitable routes of administering a compound of the present invention in vivo and in vitro are well known in the art and may be selected by those of ordinary skill in the art.

In one embodiment, the pharmaceutical composition of the present invention is administered parenterally; i.e. by a mode of administration other than enteral and topical administration; usually by injection, and include epidermal, intravenous, intramuscular, intra-arterial, intrathecal, intracapsular, intra-orbital, intracardiac, intradermal, intraperitoneal, intratendinous, transtracheal, subcutaneous, subcuticular, intra-articular, subcapsular, subarachnoid, intraspinal, intracranial, intrathoracic, epidural and intrasternal injection and infusion. In particular, the pharmaceutical composition of the present invention may be administered by intravenous or subcutaneous injection or infusion.

Uses and Therapeutic Applications

The present invention further provides an antibody, such as a bispecific antibody as defined herein for use as a medicament. The anti-ROR2 antibodies of the present invention can be used in the treatment or prevention of a disease or disorder involving cells expressing ROR2 in particular on the surface of the cells. In particular, the bispecific antibodies according to the invention; i.e. antibodies which comprise antigen binding regions capable of binding ROR2 and CD3 may be useful in therapeutic settings in which specific targeting and T cell-mediated killing of cells that express ROR2 is desired, and they may be more efficient compared to a regular anti-ROR2 antibody in certain such indications and settings.

In one embodiment, the antibody, such as the bispecific antibody of the present invention is disclosed herein for use in the treatment of cancer. The antibody, such as the bispecific antibody may in particular be use in treatment of a cancer, wherein the cancer is characterized by expression of ROR2 in at least some of the tumor cells. In one embodiment the antibody of the invention is for use in the treatment of a cancer which is a solid tumor.

The cancer may in particular be selected from the group comprising sarcomas, fibrosarcoma, gastro-intestinal stromal tumors, leiomyosarcoma, rhabdomyosarcoma, liposarcoma, uterine cancer, lung cancer, pancreatic cancer, renal cancer, colorectal cancer, cervical cancer and breast cancer.

Additionally, the invention relates to the use of an antibody according to the invention for the manufacture of a medicament, such as a medicament for the treatment of cancer, e.g. a cancer selected from the group comprising sarcomas, fibrosarcoma, gastro-intestinal stromal tumors, leiomyosarcoma, rhabdomyosarcoma, liposarcoma, uterine cancer, lung cancer, pancreatic cancer, renal cancer, colorectal cancer, cervical cancer and breast cancer.

In a further aspect, the invention provides method of treating a disease, the method comprising administering an antibody such as a bispecific antibody, a composition, such as a pharmaceutical composition according to the invention to a subject in need thereof.

In particular embodiments of the invention, said method is for treatment of a cancer. The method of the invention may in particular comprise the steps of:
a) selecting a subject suffering from a cancer comprising tumor cells expressing ROR2 and/or a cancer known to express ROR2; and
b) administering to the subject the antibody, such as the bispecific antibody, or the pharmaceutical composition of the present invention.

The cancer may in particular be selected from the group comprising of sarcomas, fibrosarcoma, gastro-intestinal stromal tumors, leiomyosarcoma, rhabdomyosarcoma, liposarcoma, uterine cancer, lung cancer, pancreatic cancer, renal cancer, colorectal cancer, cervical cancer and breast cancer.

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage.

The efficient dosages and the dosage regimens for the antibodies depend on the disease or condition to be treated and may be determined by the persons skilled in the art. An exemplary, non-limiting range for a therapeutically effective amount of a compound of the present invention is about 0.001-10 mg/kg, such as about 0.001-5 mg/kg, for example about 0.001-2 mg/kg, such as about 0.001-1 mg/kg, for instance about 0.001, about 0.01, about 0.1, about 1 or about 10 mg/kg. Another exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3, about 5, or about 8 mg/kg.

A physician having ordinary skill in the art may readily determine and prescribe the effective amount of the pharmaceutical composition required. For example, the physician or veterinarian could start doses of the antibody employed in the pharmaceutical composition at levels lower than that required to achieve the desired therapeutic effect and gradually increase the dosage until the desired effect is achieved. In general, a suitable daily dose of an antibody of the present invention will be that amount of the compound which is the lowest dose effective to produce a therapeutic effect. Administration may e.g. be parenteral, such as intravenous, intramuscular or subcutaneous.

The antibody may also be administered prophylactically to reduce the risk of developing cancer, delay the onset of the occurrence of an event in cancer progression, and/or reduce the risk of recurrence when a cancer is in remission.

The antibodies of the invention may also be administered in combination therapy, i.e., combined with other therapeutic agents relevant for the disease or condition to be treated. Accordingly, in one embodiment, the antibody-containing medicament is for combination with one or more further therapeutic agents, such as a cytotoxic, chemotherapeutic or anti-angiogenic agent.

Antibody Production

Also provided herein is a method for producing the antibody, such as the bispecific antibody of the invention.

There is provided a method for producing the antibody of the invention, comprising the steps of
a. culturing a host cell comprising an expression vector as defined herein; and
b. and purifying said antibody from the culture medium.

In another embodiment of the invention, wherein the antibody comprises a binding region capable of binding to ROR2 and a binding region capable of binding to CD3, the antibody may be produced using a method comprising the steps of
a. providing an antibody capable of binding to ROR2, said antibody comprising an antigen-binding region capable of binding to ROR2 as defined herein above;
b. providing an antibody capable of binding to CD3, said antibody comprising an antigen-binding region capable of binding to CD3 as defined herein above;
c. incubating said antibody capable of binding to ROR2 together with said antibody capable of binding to CD3 under reducing conditions sufficient to allow cysteines in the hinge region to undergo disulfide-bond isomerization, and
d. obtaining said antibody capable of binding to ROR2 and CD3.

In a further embodiment the method for producing an antibody capable of binding to both ROR2 and CD3, steps a) and/or b) above further comprise:
providing cells containing expression vectors for producing said antibody or said antibodies; and
allowing the cells to produce said antibody or said antibodies and subsequently,
obtaining said antibody or said antibodies, thereby providing said antibody or said antibodies.

Kits

The invention further provides a kit-of-parts comprising an antibody as disclosed above, such as a kit for use as a companion diagnostic for identifying within a population of patients, those patients which have a propensity to respond to treatment with an antibody as defined herein above or for predicting efficacy or anti-tumor activity of said antibody or immunoconjugate or ADC when used in treatment of a patient, the kit comprising an antibody as defined above; and instructions for use of said kit.

Anti-Idiotypic Antibodies

In a further aspect, the invention relates to an anti-idiotypic antibody which binds to an antibody comprising at least one antigen-binding region capable of binding to ROR2, i.e. an antibody according to the invention as described herein. In particular embodiments, the anti-idiotypic antibody binds to the antigen-binding region capable of binding to ROR2.

An anti-idiotypic (Id) antibody is an antibody which recognizes unique determinants generally associated with the antigen-binding site of an antibody. An anti Id antibody may be prepared by immunizing an animal of the same species and genetic type as the source of an anti-ROR2 monoclonal antibody with the monoclonal antibody against which an anti-Id is being prepared. The immunized animal typically can recognize and respond to the idiotypic determinants of the immunizing antibody by producing an antibody to these idiotypic determinants (the anti-Id antibody). Such antibodies are described in for instance U.S. Pat. No. 4,699,880. Such antibodies are further features of the present invention.

An anti-Id antibody may also be used as an “immunogen” to induce an immune response in yet another animal, producing a so-called anti-anti-Id antibody. An anti-anti-Id antibody may be epitopically identical to the original monoclonal antibody, which induced the anti-Id antibody. Thus, by using antibodies to the idiotypic determinants of a monoclonal antibody, it is possible to identify other clones expressing antibodies of identical specificity. Anti-Id antibodies may be varied (thereby producing anti-Id antibody variants) and/or derivatized by any suitable technique, such as those described elsewhere herein with respect to ROR2 specific antibodies of the present invention. For example, a monoclonal anti-Id antibody may be coupled to a carrier such as keyhole limpet hemocyanin (KLH) and used to immunize BALB/c mice. Sera from these mice typically will contain anti-anti-Id antibodies that have the binding properties similar, if not identical, to an original/parental anti-ROR2 antibody.

TABLE 1
Sequences

| SEQ ID | | | |
|---|---|---|---|
| NO | Identifier | Domain | Amino acid sequence |
| SEQ ID | Human | ORF | MARGSALPRRPLLCIPAVWAAAALLLSVSRTSGEVEVLDPNDPLGPLDGQ |
| NO: 1 | ROR2 | | DGPIPTLKGYFLNFLEPVNNITIVQGQTAILHCKVAGNPPPNVRWLKNDAP |
| | | | VVQEPRRIIIRKTEYGSRLRIQDLDTTDTGYYQCVATNGMKTITATGVLFVR |
| | | | LGPTHSPNHNFQDDYHEDGFCQPYRGIACARFIGNRTIYVDSLQMQGEIE |
| | | | NRITAAFTMIGTSTHLSDQCSQFAIPSFCHFVFPLCDARSRTPKPRELCRDE |
| | | | CEVLESDLCRQEYTIARSNPLILMRLQLPKCEALPMPESPDAANCMRIGIPA |
| | | | ERLGRYHQCYNGSGMDYRGTASTTKSGHQCQPWALQHPHSHHLSSTD |
| | | | FPELGGGHAYCRNPGGQMEGPWCFTQNKNVRMELCDVPSCSPRDSSK |
| | | | MGILYILVPSIAIPLVIACLFFLVCMCRNKQKASASTPQRRQLMASPSQDM |
| | | | EMPLINQHKQAKLKEISLSAVRFMEELGEDRFGKVYKGHLFGPAPGEQTQ |
| | | | AVAIKTLKDKAEGPLREEFRHEAMLRARLQHPNVVCLLGVVTKDQPLSMIF |
| | | | SYCSHGDLHEFLVMRSPHSDVGSTDDDRTVKSALEPPDFVHLVAQIAAG |
| | | | MEYLSSHHVVHKDLATRNVLVYDKLNVKISDLGLFREVYAADYYKLLGNSL |
| | | | LPIRWMAPEAIMYGKFSIDSDIWSYGVVLWEVFSYGLQPYCGYSNQDVVE |
| | | | MIRNRQVLPCPDDCPAWVYALMIECWNEFPSRRPRFKDIHSRLRAWGNL |
| | | | SNYNSSAQTSGASNTTQTSSLSTSPVSNVSNARYVGPKQKAPPFPQPQFIP |
| | | | MKGQIRPMVPPPQLYVPVNGYQPVPAYGAYLPNFYPVQIPMQMAPQQ |
| | | | VPPQMVPKPSSHHSGSGSTSTGYVTTAPSNTSMADRAALLSEGADDTQN |
| | | | APEDGAQSTVQEAEEEEEGSVPETELLGDCDTLQVDEAQVQLEA |
| SEQ ID | ROR2- | VH | QSVEESGGRLVTPGTPLTLTCTVSGFSLSSYSMSWVRQAPGKGLEWIGVIY |
| NO: 2 | A_VH | | VNSKTWYANWAKGRFTISKTSTTVDLKITSPTTEDTATYFCARGDAGYTT |
| | | | NSWLWGPGTLVTVSS |
| SEQ ID | ROR2- | VH_ | GFSLSSYS |
| NO: 3 | A_VH- | CDR1 | |
| | CDR1 | | |
| SEQ ID | ROR2- | VH_ | IYVNSKT |
| NO: 4 | A_VH- | CDR2 | |
| | CDR2 | | |
| SEQ ID | ROR2- | VH_ | ARGDAGYTTNSWL |
| NO: 5 | A_VH- | CDR3 | |
| | CDR3 | | |
| SEQ ID | ROR2- | VL | AFELTQTPASVSEPVGGTVTIKCQASESIDNYCSWYQQKPGQPPKLLIYQA |
| NO: 6 | A_VL | | FKLASGVPSRFRGSGSGTQFTLTISDLESDDAATYYCQSYSGISTTAFGGGT |
| | | | EVVVK |
| SEQ ID | ROR2- | VL_ | ESIDNY |
| NO: 7 | A_VL- | CDR1 | |
| | CDR1 | | |
| SEQ ID | ROR2- | VL_ | QAF |
| NO: 8 | A_VL- | CDR2 | |
| | CDR2 | | |
| SEQ ID | ROR2- | VL_ | QSYSGISTTA |
| NO: 9 | A_VL- | CDR3 | |
| | CDR3 | | |
| SEQ ID | ROR2- | VH | QSVLESGGGLVQPGGSLRLSCTVSGFSLSSYSMSWVRQAPGKGLEWIGVI |
| NO: 10 | A_HC1 | | YVNSKTWYANWAKGRFTISKTSTTVDLKITSPTAEDTATYFCARGDAGYT |
| | | | TNSWLWGQGTLVTVSS |
| SEQ ID | ROR2- | VH | QSVLESGGGLVQPGGSLRLSCTVSGFSLSSYSMSWVRQAPGKGLEWIGVI |
| NO: 11 | A_HC2 | | YVNSKTWYANWAKGRFTISKTSTTVYLQMNSLRAEDTATYFCARGDAGY |
| | | | TTNSWLWGQGTLVTVSS |
| SEQ ID | ROR2- | VH | EVQLLESGGGLVQPGGSLRLSCTVSGFSLSSYSMSWVRQAPGKGLEWIGV |
| NO: 12 | A_HC3 | | IYVNSKTWYANWAKGRFTISKTSTTVYLQMNSLRAEDTATYYCARGDAG |
| | | | YTTNSWLWGQGTLVTVSS |
| SEQ ID | ROR2- | VH | EVQLLESGGGLVQPGGSLRLSCTVSGFSLSSYSMSWVRQAPGKGLEWIGV |
| NO: 13 | A_HC4 | | IYVNSKTWYANWAKGRFTISKDNSKNTVYLQMNSLRAEDTATYYCARGD |
| | | | AGYTTNSWLWGQGTLVTVSS |
| SEQ ID | ROR2- | VH | EVQLLESGGGLVQPGGSLRLSCAASGFSLSSYSMSWVRQAPGKGLEWIG |
| NO: 14 | A_HC5 | | VIYVNSKTWYANWAKGRFTISKDNSKNTVYLQMNSLRAEDTATYYCARG |
| | | | DAGYTTNSWLWGQGTLVTVSS |
| SEQ ID | ROR2- | VH | EVQLLESGGGLVQPGGSLRLSCTVSGFSLSSYSMSWVRQAPGKGLEWIGV |
| NO: 15 | A_HC6 | | IYVNSKTWYADSVKGRFTISKDNSKNTVYLQMNSLRAEDTATYYCARGDA |
| | | | GYTTNSWLWGQGTLVTVSS |
| SEQ ID | ROR2- | VH | EVQLLESGGGLVQPGGSLRLSCAASGFSLSSYSMSWVRQAPGKGLEWIG |
| NO: 16 | A_HC7 | | VIYVNSKTWYADSVKGRFTISKDNSKNTVYLQMNSLRAEDTATYYCARGD |
| | | | AGYTTNSWLWGQGTLVTVSS |
| SEQ ID | ROR2- | VL | AFELTQSPSSLSASVGDRVTITCQASESIDNYCSWYQQKPGKAPKLLIYQAF |
| NO: 17 | A_LC1 | | KLASGVPSRFRGSGSGTDFTLTISSLQSEDFATYYCQSYSGISTTAFGGGTE |
| | | | VVIK |
| SEQ ID | ROR2- | VL | AFELTQSPSSLSASVGDRVTITCQASESIDNYLSWYQQKPGKAPKLLIYQAF |
| NO: 18 | A_LC2 | | KLASGVPSRFRGSGSGTDFTLTISSLQSEDFATYYCQSYSGISTTAFGGGTE |
| | | | VVIK |
| SEQ ID | ROR2- | VL | AFELTQSPSSLSASVGDRVTITCQASESIDNYLSWYQQKPGKAPKLLIYQAF |
| NO: 19 | A_ LC3 | | KLASGVPSRFRGSGSGTDFTLTISSLOPEDFATYYCQSYSGISTTAFGGGTK |
| | | | VEIK |
| SEQ ID | ROR2- | VL | DIQLTQSPSSLSASVGDRVTITCQASESIDNYLSWYQQKPGKAPKLLIYQAF |
| NO: 20 | A_LC4 | | KLASGVPSRFRGSGSGTDFTLTISSLOPEDFATYYCQSYSGISTTAFGGGTK |
| | | | VEIK |
| SEQ ID | human | Mature | QDGNEEMGGITQTPYKVSISGTTVILTCPQYPGSEILWQHNDKNIGGDED |
| NO: 21 | CD38 | protein | DKNIGSDEDHLSLKEFSELEQSGYYVCYPRGSKPEDANFYLYLRARVCENC |
| | (epsilon) | | MEMDVMSVATIVIVDICITGGLLLLVYYWSKNRKAKAKPVTRGAGAGGR |
| | | | QRGQNKERPPPVPNPDYEPIRKGQRDLYSGLNQRRI |
| SEQ ID | CD3- | VH | EVKLLESGGGLVQPKGSLKLSCAASGFTFNTYAMNWVRQAPGKGLEWVA |
| NO: 22 | SP34_ | | RIRSKYNNYATYYADSVKDRFTISRDDSQSILYLQMNNLKTEDTAMYYCVR |
| | VH WT | | HGNFGNSYVSWFAYWGQGTLVTVSA |
| SEQ ID | CD3- | VH_ | GFTFNTYA |
| NO: 23 | SP34_ | CDR1 | |
| | VH-CDR1 | | |
| SEQ ID | CD3- | VH_ | IRSKYNNYAT |
| NO: 24 | SP34_ | CDR2 | |
| | VH-CDR2 | | |
| SEQ ID | CD3- | VH_ | VRHGNFGNSYVSWFAY |
| NO: 25 | SP34_ | CDR3 | |
| | VH-CDR3 | | |
| SEQ ID | CD3- | VL | QAVVTQESALTTSPGETVTLTCRSSTGAVTTSNYANWVQEKPDHLFTGLI |
| NO: 26 | SP34_VL | | GGTNKRAPGVPARFSGSLIGDKAALTITGAQTEDEAIYFCALWYSNLWVF |
| | WT | | GGGTKLTVL |
| SEQ ID | CD3- | VL_ | TGAVTTSNY |
| NO: 27 | SP34_VL- | CDR1 | |
| | CDR1 | | |
| No | CD3- | VL_ | GTN |
| number | SP34_VL- | CDR2 | |
| (only 3 | CDR2 | | |
| aa) | (GTN) | | |
| SEQ ID | CD3- | VL_ | ALWYSNLWV |
| NO: 28 | SP34_VL- | CDR3 | |
| | CDR3 | | |
| SEQ ID | huCD3- | VH | EVKLVESGGGLVQPGGSLRLSCAASGFTFNTYAMNWVRQAPGKGLEWV |
| NO: 29 | H1L1_ | | ARIRSKYNNYATYYADSVKDRFTISRDDSKSSLYLQMNNLKTEDTAMYYCV |
| | VH | | RHGNFGNSYVSWFAYWGQGTLVTVSS |
| SEQ ID | huCD3- | VL_ | QAVVTQEPSFSVSPGGTVTLTCRSSTGAVTTSNYANWVQQTPGQAFRGL |
| NO: 30 | H1L1_ | CDR3 | IGGTNKRAPGVPARFSGSLIGDKAALTITGAQADDESIYFCALWYSNLWVF |
| | VL | | GGGTKLTVL |
| SEQ ID | huCD3- | VH_ | VRGGNFGNSYVSWFAY |
| NO: 31 | H1L1- | CDR3 | |
| | H101G_ | | |
| SEQ ID | huCD3- | VH | EVKLVESGGGLVQPGGSLRLSCAASGFTFNTYAMNWVRQAPGKGLEWV |
| NO: 32 | H1L1- | | ARIRSKYNNYATYYADSVKDRFTISRDDSKSSLYLQMNNLKTEDTAMYYCV |
| | H101G_ | | RGGNFGNSYVSWFAYWGQGTLVTVSS |
| | VH | | |
| SEQ ID | Human | Constant | ASTKGPSVFPLAPSSKSTSGGTAALGCLVKDYFPEPVTVSWNSGALTSGVH |
| NO: 33 | IgG1-Fc | | TFPAVLQSSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKEYKC |
| | | | KVSNKALPAPIEKTISKAKGQPREPQVYTLPPSREEMTKNQVSLTCLVKGFY |
| | | | PSDIAVEWESNGQPENNYKTTPPVLDSDGSFFLYSKLTVDKSRWQQGNVF |
| | | | SCSVMHEALHNHYTQKSLSLSPGK |
| SEQ ID | Human | Constant | ASTKGPSVFPLAPSSKSTSGGTAALGCLVKDYFPEPVTVSWNSGALTSGVH |
| NO: 34 | IgG1-Fc- | | TFPAVLQSSGLYSLSSVVTVPSSSLGTQTYICNVNHKPSNTKVDKRVEPKSC |
| | FEAR | | DKTHTCPPCPAPEFEGGPSVFLFPPKPKDTLMISRTPEVTCVVVAVSHEDP |
| | | | EVKFNWYVDGVEVHNAKTKPREEQYNSTYRVVSVLTVLHQDWLNGKEYK |
| | | | CKVSNKALPAPIEKTISKAKGQPREPQVYTLPPSREEMTKNQVSLTCLVKGF |
| | | | YPSDIAVEWESNGQPENNYKTTPPVLDSDGSFFLYSRLTVDKSRWQQGN |
| | | | VFSCSVMHEALHNHYTQKSLSLSPGK |
| SEQ ID | Human | Constant | ASTKGPSVFPLAPSSKSTSGGTAALGCLVKDYFPEPVTVSWNSGALTSGVH |
| NO: 35 | IgG1-Fc- | | TFPAVLQSSGLYSLSSVVTVPSSSLGTQTYICNVNHKPSNTKVDKRVEPKSC |
| | FEAL | | DKTHTCPPCPAPEFEGGPSVFLFPPKPKDTLMISRTPEVTCVVVAVSHEDP |
| | | | EVKFNWYVDGVEVHNAKTKPREEQYNSTYRVVSVLTVLHQDWLNGKEYK |
| | | | CKVSNKALPAPIEKTISKAKGQPREPQVYTLPPSREEMTKNQVSLTCLVKGF |
| | | | YPSDIAVEWESNGQPENNYKTTPPVLDSDGSFLLYSKLTVDKSRWQQGNV |
| | | | FSCSVMHEALHNHYTQKSLSLSPGK |
| SEQ ID | Human | Constant | RTVAAPSVFIFPPSDEQLKSGTASVVCLLNNFYPREAKVQWKVDNALQSG |
| NO: 36 | kappa | | NSQESVTEQDSKDSTYSLSSTLTLSKADYEKHKVYACEVTHQGLSSPVTKSF |
| | LC | | NRGEC |
| SEQ ID | Human | Constant | GQPKAAPSVTLFPPSSEELQANKATLVCLISDFYPGAVTVAWKADSSPVKA |
| NO: 37 | lambda | | GVETTTPSKQSNNKYAASSYLSLTPEQWKSHRSYSCQVTHEGSTVEKTVAP |
| | LC | | TECS |
| SEQ ID | IgG1- | Constant | ASTKGPSVFPLAPSSKSTSGGTAALGCLVKDYFPEPVTVSWNSGALTSGVH |
| NO: 38 | F405L | | TFPAVLQSSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKEYKC |
| | | | KVSNKALPAPIEKTISKAKGQPREPQVYTLPPSREEMTKNQVSLTCLVKGFY |
| | | | PSDIAVEWESNGQPENNYKTTPPVLDSDGSFLLYSKLTVDKSRWQQGNVF |
| | | | SCSVMHEALHNHYTQKSLSLSPGK |
| SEQ ID | cynomolgus | ORF | MARGSALPRRPLLCIPAVWAAAALLLSVSRTSGEVEVPDPNDPLGPLDGQ |
| NO: 39 | monkey | | DGPIPTLKGYFLNFLEPVNNITIVQGQTAILHCKVAGNPPPNVRWLKNDAP |
| | (Macaca | | VVQEPRRIIIRKTEYGSRLRIQDLDTTDTGYYQCVATNGMKTITATGVLFVR |
| | fascicularis) | | LGPTHSPNHNFQDDYHEDGFCQPYRGIACARFIGNRTIYVDSLQMQGEIE |
| | ROR2 | | NRITAAFTMIGTSTHLSDQCSQFAIPSFCHFVFPLCDARSRAPKPRELCRDE |
| | | | CEVLESDLCRQEYTIARSNPLILMRLQLPKCEALPMPESPDAANCMRIGIPA |
| | | | ERLGRYHQCYNGSGTDYRGTASTTKSGHQCQPWALQHPHSHHLSSTDFP |
| | | | ELGGGHAYCRNPGGQMEGPWCFTQNKNVRMELCDVPSCSPRDSSKMG |
| | | | ILYILVPSIAIPLVIACLFFLVCMCRNKQKASASTPQRRQLMASPSQDMEMP |
| | | | LINQHKQAKLKEISLSAVRFMEELGEDRFGKVYKGHLFGPAPGEQTQAVAI |
| | | | KTLKDKAEGPLREEFRHEAMLRARLQHPNIVCLLGVVTKDQPLSMIFSYCS |
| | | | HSDLHEFLVMRSPHSDVGSTDDDRTVKSALEPPDFVHLVAQIAAGMEYLS |
| | | | SHHVVHKDLATRNVLVYDKLNVKISDLGLFREVYAADYYKLLGNSLLPIRW |
| | | | MAPEAIMYGKFSIDSDIWSYGVVLWEVFSYGLQPYCGYSNQDVVEMIRN |
| | | | RQVLPCPDDCPAWVYALMIECWNEFPSRRPRFKDIHSRLRAWGNLSNYN |
| | | | SSAQTSGASNTTQTSSLSTSPVSNVSNARYMGPKQKAPPFPQPQFIPMKG |
| | | | QIRPMVPPPQLYIPVNGYQPVPAYGAYLPNFYPVQIPMQMAPQQVPPQ |
| | | | MVPKPSSHHSGSGSTSTGYVTTAPSNTSVADRAALLSEGTEDAQNAPEDG |
| | | | AQSPVQEAEEEEGSVPETELLGDSDTLQVDEAQVQLEA |
| SEQ ID | Human | ORF | MHRPRRRGTRPPLLALLAALLLAARGAAAQETELSVSAELVPTSSWNISSEL |
| NO: 40 | ROR1 | | NKDSYLTLDEPMNNITTSLGQTAELHCKVSGNPPPTIRWFKNDAPVVQEP |
| | | | RRLSFRSTIYGSRLRIRNLDTTDTGYFQCVATNGKEVVSSTGVLFVKFGPPPT |
| | | | ASPGYSDEYEEDGFCQPYRGIACARFIGNRTVYMESLHMQGEIENQITAAF |
| | | | TMIGTSSHLSDKCSQFAIPSLCHYAFPYCDETSSVPKPRDLCRDECEILENVL |
| | | | CQTEYIFARSNPMILMRLKLPNCEDLPQPESPEAANCIRIGIPMADPINKNH |
| | | | KCYNSTGVDYRGTVSVTKSGRQCQPWNSQYPHTHTFTALRFPELNGGHS |
| | | | YCRNPGNQKEAPWCFTLDENFKSDLCDIPACDSKDSKEKNKMEILYILVPS |
| | | | VAIPLAIALLFFFICVCRNNQKSSSAPVQRQPKHVRGQNVEMSMLNAYKP |
| | | | KSKAKELPLSAVRFMEELGECAFGKIYKGHLYLPGMDHAQLVAIKTLKDYN |
| | | | NPQQWTEFQQEASLMAELHHPNIVCLLGAVTQEQPVCMLFEYINQGDL |
| | | | HEFLIMRSPHSDVGCSSDEDGTVKSSLDHGDFLHIAIQIAAGMEYLSSHFFV |
| | | | HKDLAARNILIGEQLHVKISDLGLSREIYSADYYRVQSKSLLPIRWMPPEAI |
| | | | MYGKFSSDSDIWSFGVVLWEIFSFGLQPYYGFSNQEVIEMVRKRQLLPCSE |
| | | | DCPPRMYSLMTECWNEIPSRRPRFKDIHVRLRSWEGLSSHTSSTTPSGGN |
| | | | ATTQTTSLSASPVSNLSNPRYPNYMFPSQGITPQGQIAGFIGPPIPQNQRFI |
| | | | PINGYPIPPGYAAFPAAHYQPTGPPRVIQHCPPPKSRSPSSASGSTSTGHVT |
| | | | SLPSSGSNQEANIPLLPHMSIPNHPGGMGITVFGNKSQKPYKIDSKQASLL |
| | | | GDANIHGHTESMISAEL |
| SEQ ID | ROR2mf- | ORF | MARGSALPRRPLLCIPAVWAAAALLLSVSRTSGEVEVPDPNDPLGPLDGQ |
| NO: 41 | T322M | | DGPIPTLKGYFLNFLEPVNNITIVQGQTAILHCKVAGNPPPNVRWLKNDAP |
| | | | VVQEPRRIIIRKTEYGSRLRIQDLDTTDTGYYQCVATNGMKTITATGVLFVR |
| | | | LGPTHSPNHNFQDDYHEDGFCQPYRGIACARFIGNRTIYVDSLQMQGEIE |
| | | | NRITAAFTMIGTSTHLSDQCSQFAIPSFCHFVFPLCDARSRAPKPRELCRDE |
| | | | CEVLESDLCRQEYTIARSNPLILMRLQLPKCEALPMPESPDAANCMRIGIPA |
| | | | ERLGRYHQCYNGSGMDYRGTASTTKSGHQCQPWALQHPHSHHLSSTDF |
| | | | PELGGGHAYCRNPGGQMEGPWCFTQNKNVRMELCDVPSCSPRDSSKM |
| | | | GILYILVPSIAIPLVIACLFFLVCMCRNKQKASASTPQRRQLMASPSQDME |
| | | | MPLINQHKQAKLKEISLSAVRFMEELGEDRFGKVYKGHLFGPAPGEQTQA |
| | | | VAIKTLKDKAEGPLREEFRHEAMLRARLQHPNIVCLLGVVTKDQPLSMIFSY |
| | | | CSHSDLHEFLVMRSPHSDVGSTDDDRTVKSALEPPDFVHLVAQIAAGMEY |
| | | | LSSHHVVHKDLATRNVLVYDKLNVKISDLGLFREVYAADYYKLLGNSLLPIR |
| | | | WMAPEAIMYGKFSIDSDIWSYGVVLWEVFSYGLQPYCGYSNQDVVEMIR |
| | | | NRQVLPCPDDCPAWVYALMIECWNEFPSRRPRFKDIHSRLRAWGNLSNY |
| | | | NSSAQTSGASNTTQTSSLSTSPVSNVSNARYMGPKQKAPPFPQPQFIPMK |
| | | | GQIRPMVPPPQLYIPVNGYQPVPAYGAYLPNFYPVQIPMQMAPQQVPP |
| | | | QMVPKPSSHHSGSGSTSTGYVTTAPSNTSVADRAALLSEGTEDAQNAPED |
| | | | GAQSPVQEAEEEEGSVPETELLGDSDTLQVDEAQVQLEA |
| SEQ ID | ROR112 | shuffle | MHRPRRRGTRPPLLALLAALLLAARGAAAQETELSVSAELVPTSSWNISSEL |
| NO: 42 | | | NKDSYLTLDEPMNNITTSLGQTAELHCKVSGNPPPTIRWFKNDAPVVQEP |
| | | | RRLSFRSTIYGSRLRIRNLDTTDTGYFQCVATNGKEVVSSTGVLFVKFGPPPT |
| | | | ASPGYSDEYEEDGFCQPYRGIACARFIGNRTVYMESLHMQGEIENQITAAF |
| | | | TMIGTSSHLSDKCSQFAIPSLCHYAFPYCDETSSVPKPRDLCRDECEILENVL |
| | | | CQTEYIFARSNPMILMRLKLPNCEDLPQPESPEAANCIRIGIPMADPINKNC |
| | | | YNGSGMDYRGTASTTKSGHQCQPWALQHPHSHHLSSTDFPELGGGHAY |
| | | | CRNPGGQMEGPWCFTQNKNVRMELCDVPSCSPRDSSKMGILVPSVAIPL |
| | | | AIALLFFFICVCRNNQKSSSAPVQRQPKHVRGQNVEMSMLNAYKPKSKAK |
| | | | ELPLSAVRFMEELGECAFGKIYKGHLYLPGMDHAQLVAIKTLKDYNNPQQ |
| | | | WTEFQQEASLMAELHHPNIVCLLGAVTQEQPVCMLFEYINQGDLHEFLIM |
| | | | RSPHSDVGCSSDEDGTVKSSLDHGDFLHIAIQIAAGMEYLSSHFFVHKDLA |
| | | | ARNILIGEQLHVKISDLGLSREIYSADYYRVQSKSLLPIRWMPPEAIMYGKFS |
| | | | SDSDIWSFGVVLWEIFSFGLQPYYGFSNQEVIEMVRKRQLLPCSEDCPPR |
| | | | MYSLMTECWNEIPSRRPRFKDIHVRLRSWEGLSSHTSSTTPSGGNATTQT |
| | | | TSLSASPVSNLSNPRYPNYMFPSQGITPQGQIAGFIGPPIPQNQRFIPINGY |
| | | | PIPPGYAAFPAAHYQPTGPPRVIQHCPPPKSRSPSSASGSTSTGHVTSLPSS |
| | | | GSNQEANIPLLPHMSIPNHPGGMGITVFGNKSQKPYKIDSKQASLLGDAN |
| | | | IHGHTESMISAEL |
| SEQ ID | ROR121 | shuffle | MHRPRRRGTRPPLLALLAALLLAARGAAAQETELSVSAELVPTSSWNISSEL |
| NO: 43 | | | NKDSYLTLDEPMNNITTSLGQTAELHCKVSGNPPPTIRWFKNDAPVVQEP |
| | | | RRLSFRSTIYGSRLRIRNLDTTDTGYFQCVATNGKEVVSSTGVLFVKFGPPPT |
| | | | ASPGYSDEYHEDGFCQPYRGIACARFIGNRTIYVDSLQMQGEIENRITAAFT |
| | | | MIGTSTHLSDQCSQFAIPSFCHFVFPLCDARSRTPKPRELCRDECEVLESDL |
| | | | CRQEYTIARSNPLILMRLQLPKCEALPMPESPDAANCMRIIPMADPINKNH |
| | | | KCYNSTGVDYRGTVSVTKSGRQCQPWNSQYPHTHTFTALRFPELNGGHS |
| | | | YCRNPGNQKEAPWCFTLDENFKSDLCDIPACDSKDSKEKNKMEILYILVPS |
| | | | VAIPLAIALLFFFICVCRNNQKSSSAPVQRQPKHVRGQNVEMSMLNAYKP |
| | | | KSKAKELPLSAVRFMEELGECAFGKIYKGHLYLPGMDHAQLVAIKTLKDYN |
| | | | NPQQWTEFQQEASLMAELHHPNIVCLLGAVTQEQPVCMLFEYINQGDL |
| | | | HEFLIMRSPHSDVGCSSDEDGTVKSSLDHGDFLHIAIQIAAGMEYLSSHFFV |
| | | | HKDLAARNILIGEQLHVKISDLGLSREIYSADYYRVQSKSLLPIRWMPPEAI |
| | | | MYGKFSSDSDIWSFGVVLWEIFSFGLQPYYGFSNQEVIEMVRKRQLLPCSE |
| | | | DCPPRMYSLMTECWNEIPSRRPRFKDIHVRLRSWEGLSSHTSSTTPSGGN |
| | | | ATTQTTSLSASPVSNLSNPRYPNYMFPSQGITPQGQIAGFIGPPIPQNQRFI |
| | | | PINGYPIPPGYAAFPAAHYQPTGPPRVIQHCPPPKSRSPSSASGSTSTGHVT |
| | | | SLPSSGSNQEANIPLLPHMSIPNHPGGMGITVFGNKSQKPYKIDSKQASLL |
| | | | GDANIHGHTESMISAEL |
| SEQ ID | ROR122 | shuffle | MHRPRRRGTRPPLLALLAALLLAARGAAAQETELSVSAELVPTSSWNISSEL |
| NO: 44 | | | NKDSYLTLDEPMNNITTSLGQTAELHCKVSGNPPPTIRWFKNDAPVVQEP |
| | | | RRLSFRSTIYGSRLRIRNLDTTDTGYFQCVATNGKEVVSSTGVLFVKFGPPPT |
| | | | ASPGYSDEYHEDGFCQPYRGIACARFIGNRTIYVDSLQMQGEIENRITAAFT |
| | | | MIGTSTHLSDQCSQFAIPSFCHFVFPLCDARSRTPKPRELCRDECEVLESDL |
| | | | CRQEYTIARSNPLILMRLQLPKCEALPMPESPDAANCMRIGIPAERLGRYH |
| | | | QCYNGSGMDYRGTASTTKSGHQCQPWALQHPHSHHLSSTDFPELGGGH |
| | | | AYCRNPGGQMEGPWCFTQNKNVRMELCDVPSCDSKDSKEKNKMEILYIL |
| | | | VPSVAIPLAIALLFFFICVCRNNQKSSSAPVQRQPKHVRGQNVEMSMLNA |
| | | | YKPKSKAKELPLSAVRFMEELGECAFGKIYKGHLYLPGMDHAQLVAIKTLK |
| | | | DYNNPQQWTEFQQEASLMAELHHPNIVCLLGAVTQEQPVCMLFEYINQ |
| | | | GDLHEFLIMRSPHSDVGCSSDEDGTVKSSLDHGDFLHIAIQIAAGMEYLSS |
| | | | HFFVHKDLAARNILIGEQLHVKISDLGLSREIYSADYYRVQSKSLLPIRWMPP |
| | | | EAIMYGKFSSDSDIWSFGVVLWEIFSFGLQPYYGFSNQEVIEMVRKRQLLP |
| | | | CSEDCPPRMYSLMTECWNEIPSRRPRFKDIHVRLRSWEGLSSHTSSTTPSG |
| | | | GNATTQTTSLSASPVSNLSNPRYPNYMFPSQGITPQGQIAGFIGPPIPQNQ |
| | | | RFIPINGYPIPPGYAAFPAAHYQPTGPPRVIQHCPPPKSRSPSSASGSTSTG |
| | | | HVTSLPSSGSNQEANIPLLPHMSIPNHPGGMGITVFGNKSQKPYKIDSKQA |
| | | | SLLGDANIHGHTESMISAEL |
| SEQ ID | ROR211 | shuffle | MARGSALPRRPLLCIPAVWAAAALLLSVSRTSGEVEVLDPNDPLGPLDGQ |
| NO: 45 | | | DGPIPTLKGYFLNFLEPVNNITIVQGQTAILHCKVAGNPPPNVRWLKNDAP |
| | | | VVQEPRRIIIRKTEYGSRLRIQDLDTTDTGYYQCVATVKFGPPPTASPGYSDE |
| | | | YEEDGFCQPYRGIACARFIGNRTVYMESLHMQGEIENQITAAFTMIGTSSH |
| | | | LSDKCSQFAIPSLCHYAFPYCDETSSVPKPRDLCRDECEILENVLCQTEYIFAR |
| | | | SNPMILMRLKLPNCEDLPQPESPEAANCIRIGIPMADPINKNHKCYNSTGV |
| | | | DYRGTVSVTKSGRQCQPWNSQYPHTHTFTALRFPELNGGHSYCRNPGNQ |
| | | | KEAPWCFTLDENFKSDLCDIPACDSKDSKEKNKMEILYILVPSVAIPLAIALLF |
| | | | FFICVCRNNQKSSSAPVQRQPKHVRGQNVEMSMLNAYKPKSKAKELPLS |
| | | | AVRFMEELGECAFGKIYKGHLYLPGMDHAQLVAIKTLKDYNNPQQWTEF |
| | | | QQEASLMAELHHPNIVCLLGAVTQEQPVCMLFEYINQGDLHEFLIMRSPH |
| | | | SDVGCSSDEDGTVKSSLDHGDFLHIAIQIAAGMEYLSSHFFVHKDLAARNIL |
| | | | IGEQLHVKISDLGLSREIYSADYYRVQSKSLLPIRWMPPEAIMYGKFSSDSDI |
| | | | WSFGVVLWEIFSFGLQPYYGFSNQEVIEMVRKRQLLPCSEDCPPRMYSLM |
| | | | TECWNEIPSRRPRFKDIHVRLRSWEGLSSHTSSTTPSGGNATTQTTSLSASP |
| | | | VSNLSNPRYPNYMFPSQGITPQGQIAGFIGPPIPQNQRFIPINGYPIPPGYA |
| | | | AFPAAHYQPTGPPRVIQHCPPPKSRSPSSASGSTSTGHVTSLPSSGSNQEA |
| | | | NIPLLPHMSIPNHPGGMGITVFGNKSQKPYKIDSKQASLLGDANIHGHTES |
| | | | MISAEL |
| SEQ ID | ROR221 | shuffle | MHRPRRRGTRPPLLALLAALLLAARGAAAQETELSVSAELVPTLKGYFLNFL |
| NO: 46 | | | EPVNNITIVQGQTAILHCKVAGNPPPNVRWLKNDAPVVQEPRRIIIRKTEY |
| | | | GSRLRIQDLDTTDTGYYQCVATNGMKTITATGVLFVRLGPTHSPNHNFQD |
| | | | DYHEDGFCQPYRGIACARFIGNRTIYVDSLQMQGEIENRITAAFTMIGTST |
| | | | HLSDQCSQFAIPSFCHFVFPLCDARSRTPKPRELCRDECEVLESDLCRQEYTI |
| | | | ARSNPLILMRLQLPKCEALPMPESPDAANCMRIIPMADPINKNHKCYNST |
| | | | GVDYRGTVSVTKSGRQCQPWNSQYPHTHTFTALRFPELNGGHSYCRNPG |
| | | | NQKEAPWCFTLDENFKSDLCDIPACDSKDSKEKNKMEILYILVPSVAIPLAIA |
| | | | LLFFFICVCRNNQKSSSAPVQRQPKHVRGQNVEMSMLNAYKPKSKAKELP |
| | | | LSAVRFMEELGECAFGKIYKGHLYLPGMDHAQLVAIKTLKDYNNPQQWTE |
| | | | FQQEASLMAELHHPNIVCLLGAVTQEQPVCMLFEYINQGDLHEFLIMRSP |
| | | | HSDVGCSSDEDGTVKSSLDHGDFLHIAIQIAAGMEYLSSHFFVHKDLAARN |
| | | | ILIGEQLHVKISDLGLSREIYSADYYRVQSKSLLPIRWMPPEAIMYGKFSSDS |
| | | | DIWSFGVVLWEIFSFGLQPYYGFSNQEVIEMVRKRQLLPCSEDCPPRMYSL |
| | | | MTECWNEIPSRRPRFKDIHVRLRSWEGLSSHTSSTTPSGGNATTQTTSLSA |
| | | | SPVSNLSNPRYPNYMFPSQGITPQGQIAGFIGPPIPQNQRFIPINGYPIPPG |
| | | | YAAFPAAHYQPTGPPRVIQHCPPPKSRSPSSASGSTSTGHVTSLPSSGSNQ |
| | | | EANIPLLPHMSIPNHPGGMGITVFGNKSQKPYKIDSKQASLLGDANIHGHT |
| | | | ESMISAEL |
| SEQ ID | b12_VH | VH | QVQLVQSGAEVKKPGASVKVSCQASGYRFSNFVIHWVRQAPGQRFEW |
| NO: 47 | | | MGWINPYNGNKEFSAKFQDRVTFTADTSANTAYMELRSLRSADTAVYYC |
| | | | ARVGPYSWDDSPQDNYYMDVWGKGTTVIVSS |
| SEQ ID | b12_VL | VL | EIVLTQSPGTLSLSPGERATFSCRSSHSIRSRRVAWYQHKPGQAPRLVIHG |
| NO: 48 | | | VSNRASGISDRFSGSGSGTDFTLTITRVEPEDFALYYCQVYGASSYTFGQG |
| | | | TKLERK |
| SEQ ID | IgG1- | Constant | ASTKGPSVFPLAPSSKSTSGGTAALGCLVKDYFPEPVTVSWNSGALTSGVH |
| NO: 49 | K409R | | TFPAVLQSSGLYSLSSVVTVPSSSLGTQTYICNVNHKPSNTKVDKRVEPKSC |
| | | | DKTHTCPPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKEYKC |
| | | | KVSNKALPAPIEKTISKAKGQPREPQVYTLPPSREEMTKNQVSLTCLVKGFY |
| | | | PSDIAVEWESNGQPENNYKTTPPVLDSDGSFFLYSRLTVDKSRWQQGNVF |
| | | | SCSVMHEALHNHYTQKSLSLSPGK |
| SEQ ID | IgG1- | Constant | ASTKGPSVFPLAPSSKSTSGGTAALGCLVKDYFPEPVTVSWNSGALTSGVH |
| NO: 50 | FEA | | TFPAVLQSSGLYSLSSVVTVPSSSLGTQTYICNVNHKPSNTKVDKRVEPKSC |
| | | | DKTHTCPPCPAPEFEGGPSVFLFPPKPKDTLMISRTPEVTCVVVAVSHEDP |
| | | | EVKFNWYVDGVEVHNAKTKPREEQYNSTYRVVSVLTVLHQDWLNGKEYK |
| | | | CKVSNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGNV |
| | | | FSCSVMHEALHNHYTQKSLSLSPGK |
| SEQ ID | CD3E27- | ORF | QDGNEEMGGITQTPYKVSISGTTVILTGGGGSGGGGSGGGGSEIVLTQSP |
| NO: 51 | GSKa | | ATLSLSPGERATLSCRASQSVSSYLAWYQQKPGQAPRLLIYDASNRATGIPA |
| | | | RFSGSGSGTDFTLTISSLEPEDFAVYYCQQRSNWPITFGQGTRLEIKRTVAA |
| | | | PSVFIFPPSDEQLKSGTASVVCLLNNFYPREAKVQWKVDNALQSGNSQES |
| | | | VTEQDSKDSTYSLSSTLTLSKADYEKHKVYACEVTHQGLSSPVTKSFNRGE |

### EXAMPLES

### Example 1—Generation of Rabbit-Human Chimeric Antibodies Specific for Human ROR2 Expression Constructs

Constructs encoding various full-length ROR2 variants were generated: human (Homo sapiens) ROR2 (Uniprot accession no. Q01974; SEQ. ID NO: 1), cynomolgus monkey (Macaca fascicularis) ROR2 (ROR2mf; Uniprot accession no. A0A2K5UT30; SEQ. ID NO: 39) and cynomolgus monkey ROR2 in which threonine at position 322 is replaced by methionine (ROR2mf-T322M; SEQ. ID NO: 41).

Also, a construct encoding full length human ROR1 (Uniprot accession no. Q01973; SEQ. ID NO: 40) was generated.

In addition, constructs encoding shuffle variants of the Ig-like domain, frizzled-like cysteine-rich domain (CRD) and kringle domain of ROR2 and ROR1 were generated:
ROR112, containing the Ig-like domain and CRD of ROR1 and the kringle domain of ROR2 (; SEQ. ID NO: 42),
ROR121, containing the Ig-like domain of ROR1, the CRD of ROR2 and the kringle domain of ROR1 (; SEQ. ID NO: 43),
ROR122, containing the Ig-like domain of ROR1, and the CRD and the kringle domain of ROR2 (; SEQ. ID NO: 44),
ROR211, containing the Ig-like domain of ROR2 and the CRD and the kringle domain of ROR1 (; SEQ. ID NO: 45),
ROR221, containing the Ig-like domain and CRD of ROR2 and the kringle domain of ROR1 (; SEQ. ID NO: 45),

as further illustrated in table 2.

TABLE 2
Shuffle variants of the Ig-like domain, frizzled-like cysteine-
rich domain (CRD) and kringle domain of ROR2 and ROR1

| | IgG-like domain | CRD | Kringle domain |
|---|---|---|---|
| Shuffle variant | origin | origin | origin |
| 112 | ROR1 | ROR1 | ROR2 |
| 121 | ROR1 | ROR2 | ROR1 |
| 122 | ROR1 | ROR2 | ROR2 |
| 211 | ROR2 | ROR1 | ROR1 |
| 221 | ROR2 | ROR2 | ROR1 |

Constructs contained suitable restriction sites for cloning and an optimal Kozak (GCCGCCACC) sequence (Kozak, M., Gene 1999; 234(2):187-208). The full length and ECD constructs were cloned in pSB, a mammalian expression vector containing Sleeping Beauty inverted terminal repeats flanking an expression cassette consisting of a CMV promoter and HSV-TK polyA signal.

Transient Expression in HEK-293F or CHO Cells

Membrane (full-length ROR2 and ROR1, SEQ. ID. Nos 1, 39, 40 and 41) proteins were transiently transfected in Freestyle 293-F cells (HEK293F, a HEK-293 subclone adapted to suspension growth and chemically defined Freestyle medium; Invitrogen, cat. no. R790-07) using 293fectin (Invitrogen, cat. no. 12347-019) essentially as described by the manufacturer, or in Freestyle CHO—S cells (CHO) (Life technologies, cat. no. R800-07) by using the Freestyle Max reagent (Life technologies, cat. no. 16447100) essentially as described by the manufacturer.

Immunization of Rabbits

Immunization of rabbits was performed at mAbDiscovery GmbH (Neuried, Germany). Rabbits were repeatedly immunized with a mixture of HEK cells overexpressing either human ROR1 (SEQ. ID. NO. 40) or human ROR2 (SEQ. ID. NO. 1). The blood of these animals was collected, and B lymphocytes were isolated. Using a MAB Discovery proprietary process, single B cells were sorted into wells of microtiter plates and further propagated. The supernatants of these single B cells were analyzed for specific binding to CHO—S cells transiently expressing human ROR2 (CHO—ROR2) or cynomolgus monkey ROR2 (CHO-mfROR2).

Recombinant Chimeric Antibody Production

Upon analyzing the primary screening results, primary hits were selected for sequencing, recombinant mAb production and purification. Variable heavy chain (VH) and light chain (VL) encoding regions were gene synthesized and cloned into mammalian expression vectors containing the human constant region-encoding sequences (Ig Kappa chain and IgG1 allotype G1m (f) heavy chain).

Recombinant rabbit-human chimeric antibodies comprising rabbit variable regions and human constant regions were produced in HEK 293 cells by transiently co-transfecting the heavy chain (HC) and light chain (LC) encoding expression vectors using an automated procedure on a Tecan Freedom Evo platform. Immunoglobulins were purified from the cell supernatant using affinity purification (Protein A) on a Dionex Ultimate 3000 HPLC system.

The produced chimeric monoclonal antibodies (mAbs) were re-analyzed for binding to CHO—ROR2 or CHO-mfROR2 cells. A total of 51 antibodies binding to both human and cynomolgus monkey ROR2 on CHO transfectants were identified. These were further analyzed for binding to the human ROR2 positive cervical cancer cell line HeLa (determined by flow cytometry, using the method described below). ROR2 binding affinity was determined using ROR2ECD-His (determined by biolayer interferometry, using the method described below), yielding a panel of 8 antibodies that showed binding in at least one assay. These eight antibodies are listed below in table 3 (of example 2) and table 4 of example 3.

### Example 2—ROR2 Binding Affinity Determination of Rabbit-Human Chimeric Antibodies Using Biolayer Interferometry

Target binding affinity of the rabbit-human chimeric antibodies was determined by label-free biolayer interferometry (BLI) on an Octet HTX instrument (ForteBio). Experiments were carried out while shaking at 1,000 RPM at 30° C.

Anti-Human IgG Fc Capture (AHC) biosensors (ForteBio, cat. no. 18-5060) were pre-conditioned by exposure to 10 mM glycine (Riedel-de Haen, cat. no. 15527) buffer pH 1.7 for 5 s, followed by neutralization in Sample Diluent (ForteBio, cat. no. 18-1048) for 5 s; both steps were repeated 5 times. Next, AHC sensors were loaded with the antibody (2.5 μg/mL in Sample diluent) for 600 s. After a baseline measurement in Sample Diluent (300 s), the association (1,000 s) and dissociation (1,000 s) of a commercially available his-tagged ROR2 extracellular domain (ROR2-ECD, G&P Biosciences, cat. no. FCL0192) was determined using a concentration range of 6.25-400 nM with two-fold dilution steps in Sample Diluent. The calculated molecular mass of ROR2 ECD based on their amino acid sequences of 42.7 kDa was used for calculations. For each antibody a reference sensor was used, which was incubated with Sample Diluent instead of antigen. AHC sensors were regenerated by exposure to 10 mM glycine buffer pH 1.7 for 5 s, followed by neutralization in Sample Diluent for 5 s; both steps were repeated twice. Subsequently sensors were loaded again with antibody for the next cycle of kinetics measurements.

Data were acquired using Data Acquisition Software v8.1.0.42 (ForteBio) and analyzed with Data Analysis Software v8.1 (ForteBio). Data traces were corrected per antibody by subtraction of the average response of the reference sensors. The Y-axis was aligned to the last 10 s of the baseline, Interstep Correction alignment to dissociation and Savitzky-Golay filtering were applied. The data was fitted with the 1:1 Global Full fit model using a window of interest for the association and dissociation times set at 1,000 s and 200 s respectively.

Table 3 shows the association rate constant kₐ (1/Ms), dissociation rate constant k_{d} (1/s) and equilibrium dissociation constant K_{D} (nM) for human ROR2-ECD of the panel of 8 rabbit-human chimeric antibodies.

TABLE 3
Binding affinities of rabbit-human chimeric ROR2 antibodies
for recombinant human ROR2-ECD (G&P Biosciences) as
determined by label-free biolayer interferometry

| | On-rate | Off-rate | |
|---|---|---|---|
| | kₐ | k_{d} | K_{D} |
| Antibody | (1/Ms) | (1/s) | (nM) |
| chIgG1-ROR2-A | 6.1E+05 | 9.5E−04 | 1.6 |
| chIgG1-ROR2-B | 2.4E+05 | 7.9E−04 | 3.3 |
| chIgG1-ROR2-C | 5.5E+05 | 2.5E−03 | 4.5 |
| chIgG1-ROR2-D | 6.3E+05 | 4.1E−03 | 6.5 |
| chIgG1-ROR2-E | 5.7E+05 | 8.0E−03 | 14 |
| chIgG1-ROR2-F | 2.5E+05 | 5.5E−03 | 22 |
| chIgG1-ROR2-G | 1.5E+05 | 3.5E−03 | 22 |
| chIgG1-ROR2-H | 1.1E+05 | 3.3E−03 | 30 |

### Example 3—Binding of Rabbit-Human Chimeric ROR2 Antibodies to ROR2 Expressed on Cervical Cancer Cell Line HeLa

Binding of rabbit-human chimeric ROR2 antibodies to ROR2 expressed on human tumor cells was determined by flow cytometry, using the ROR2 expressing cervical adenocarcinoma cell line HeLa (ATCC, cat. no. CCL-2). To confirm that binding to HeLa cells was dependent on ROR2 expression, HeLa cells in which ROR2 expression was ablated using a single guide RNA uniquely targeting the human ROR2 gene (target sequence GAAGTGGCAGAAGGATGGGA) in CRISPR (clustered regularly interspaced short palindrome repeats)-associated nuclease Cas9 based gene-editing technology (Cellecta, USA) were used.

Cells (1×10⁵ cells/well) were incubated in polystyrene 96-well round-bottom plates (Greiner bio-one, cat. no. 650180) with serial dilutions of antibodies (ranging from 0.01 to 10 μg/mL in 3- or 4-fold dilution steps) in 100 μL PBS/0.1% BSA/0.02% azide (FACS buffer) at 4° C. for 30-60 min. Experiments were performed in technical duplicate. After washing twice in FACS buffer, cells were incubated in 50 μL secondary antibody (R-Phycoerythrin [PE]-conjugated goat-anti-human IgG F(ab′)₂; diluted 1:200 in FACS buffer; Jackson ImmunoResearch Laboratories, Inc., West Grove, PA, cat. no. 109-116-098) at 4° C. for 30 min. Cells were washed twice in FACS buffer, re-suspended in 30 μL FACS buffer containing Topro-3 (1:10,000 dilution) and analyzed on an iQue Screener (Intellicyt Corporation, USA). Binding curves were analyzed using non-linear regression (sigmoidal dose-response with variable slope) using GraphPad Prism V7.02 software (GraphPad Software, San Diego, CA, USA).

From the panel of 8 rabbit-human chimeric antibodies, 7 antibodies showed low binding (maximal MFI lower than 5,000) to HeLa cells and 1 antibody, chIgG1-ROR2-A, showed high binding (maximal MFI above 20,000) to HeLa cells (Table 4). chIgG1-ROR2-A did not bind to HeLa cells in which ROR2 was specifically inactivated indicating that chIgG1-ROR2-A is ROR2-specific.

TABLE 4
Binding of rabbit-human chimeric ROR2 antibodies to HeLa cells

| | EC50 | Maximum binding |
|---|---|---|
| Antibody | (ROR2) | (MFI) |
| chIgG1-ROR2-A | 0.217 | 30,093 |
| chIgG1-ROR2-B | 0.228 | 3,818 |
| chIgG1-ROR2-C | 0.094 | 2,373 |
| chIgG1-ROR2-D | 0.001 | 1,943 |
| chIgG1-ROR2-E | 0.048 | 2,436 |
| chIgG1-ROR2-F | 0.178 | 4,310 |
| chIgG1-ROR2-G | 0.378 | 2,870 |
| chIgG1-ROR2-H | 0.384 | 1,714 |

chIgG1-ROR2-A showed minimal binding to the ROR1 expressing cell line Calu-1. This binding was not affected by ablation of ROR1 expression using a single guide RNA uniquely targeting the human ROR1 gene (target sequence: GGAGTCTTTGCACATGCAAG). Any binding of chIgG1-ROR2-A was reduced by ablation of ROR2 expression, indicating that low ROR2 expression in the Calu-1 cell line was responsible for the residual binding of chIgG1-ROR2-A to the Calu-1 cell line.

In conclusion, chIgG1-ROR2-A was the only antibody in the chimeric ROR2 specific antibody panel that showed high binding to ROR2 positive tumor cells. Binding was shown to be ROR2 specific.

### Example 4—Binding of chIgG1-ROR2-A to CHO Cells Expressing ROR1/2 Shuffle Proteins

To investigate the ROR2 domain involved in binding of the ROR2 specific antibody A, binding of chIgG1-ROR2-A to CHO cells transfected to transiently express shuffle variants of the Ig-like domain, CRD and the kringle domain of ROR2 and ROR1 was explored.
ROR112, containing the Ig-like domain and CRD of ROR1 and kringle domain of ROR2
ROR121, containing the Ig-like domain, the CRD of ROR2 and kringle domain of ROR1
ROR122, containing the Ig-like domain of ROR1 and the CRD and kringle domain of ROR2
ROR211, containing the Ig-like domain of ROR2 and the CRD and kringle domain of ROR1
ROR221, containing the Ig-like domain and CRD of ROR2 and the kringle domain of ROR1

Binding was determined by flow cytometry using a cell imaging screening system (Celllnsight, Thermo Fisher) as per manufacturer's recommendations. In short, CHO cells expressing shuffle constructs ROR112, ROR121, ROR122, ROR211 or ROR221 (3,000 cells/well in 384-well plates) were incubated with antibody or control samples for 18 h at 37° C./5% CO₂, washed and incubated with an Alexa488-labeled detection antibody for 4 h. Hoechst dye was added and fluorescence images were collected, measuring total spot intensity (RFU). As shown in Table 5, chIgG1-ROR2-A bound to cells expressing ROR112 and ROR122, but not to cells expressing ROR121, ROR211 or ROR221. This indicates that the kringle domain of ROR2 is involved in the binding of chIgG1-ROR2-A.

TABLE 5
Binding of chIgG1-ROR2-A to CHO cells expressing ROR1/2 shuffle
proteins. Yes: RFU higher than 12,000; no: RFU = 0.

| CHO cell | | | | | |
|---|---|---|---|---|---|
| expressing | ROR112 | ROR121 | ROR122 | ROR211 | ROR221 |
| Binding | yes | no | yes | no | no |
| (yes/no) | | | | | |

### Example 5—Humanization of Rabbit Chimeric Antibodies

Generation of Humanized Antibody Sequences

Humanized antibody sequences derived from antibody chIgG1-ROR2-A were generated at Abzena (Cambridge, UK). Humanized antibody sequences were generated using germline humanization (CDR-grafting) technology. Humanized V region genes were designed based upon human germline sequences with closest homology to the VH and Vκ amino acid sequences of the rabbit antibody. A series of seven VH and four Vκ (VL) germline humanized V-region genes were designed and named according to the below table 6:

| VH | VL |
|---|---|
| ROR2-A-HC1 | ROR2-A-LC1 |
| ROR2-A-HC2 | ROR2-A-LC2 |
| ROR2-A-HC3 | ROR2-A-LC3 |
| ROR2-A-HC4 | ROR2-A-LC4 |
| ROR2-A-HC5 | |
| ROR2-A-HC6 | |
| ROR2-A-HC7 | |

Structural models of the rabbit antibody V regions were produced using Swiss PDB and analyzed in order to identify amino acids in the V region frameworks that may be important for the binding properties of the antibody. These amino acids were noted for incorporation into one or more variant CDR-grafted antibodies.

The heavy and light chain V region amino acid sequence were compared against a database of human germline V and J segment sequences in order to identify the heavy and light chain human sequences with the greatest degree of homology for use as human variable domain frameworks. The germline sequences used as the basis for the humanized designs are shown in Table 7.

TABLE 7
Closest matching human germline V
segment and J segment sequences.

| | |
|---|---|
| Heavy chain | Light chain (κ) |

| Human V | Human J | Human V | Human J |
|---|---|---|---|
| region | region | region | region |
| germline | germline | germline | germline |
| segment | segment | segment | segment |
| IGHV3-23*03 | IGHJ2 | IGKV1-39*01 | IGKJ4 |

A series of humanized heavy and light chain V regions were then designed by grafting the CDRs onto the frameworks and, if necessary, by back-mutating residues which may be critical for the antibody binding properties, as identified in the structural modelling, to rabbit residues. Variant sequences with the lowest incidence of potential T cell epitopes were then selected using Abzena's proprietary in silico technologies, iTope™ and TCED™ (T Cell Epitope Database) (Perry, L. C. A, Jones, T. D. and Baker, M. P. New Approaches to Prediction of Immune Responses to Therapeutic Proteins during Preclinical Development (2008). Drugs in R&D 9 (6): 385-396; Bryson, C. J., Jones, T. D. and Baker, M. P. Prediction of Immunogenicity of Therapeutic Proteins (2010). Biodrugs 24 (1):1-8). Finally, the nucleotide sequences of the designed variants were codon optimized.

The variable region sequences of the humanized ROR2 antibodies are shown in Table 1.

The obtained sequences of variable regions of heavy and light chains were gene synthesized and each possible combination of heavy and light chain was cloned into an expression vector including a human IgG1 heavy chain containing the following amino acid mutations: L234F, L235E, D265A (FEA mutations, for silencing of the FcγR and C1q binding; Engelberts et al, 2020, EBioMedicine 52: 102625) and K409R (R), together indicated as FEAR, wherein the amino acid position number is according to Eu numbering (correspond to SEQ ID NO 34), and into expression vectors including human kappa or lambda light chain.

### Example 6—ROR2 Binding Affinity Determination of Humanized Variants of chIgG1-ROR2-A Using Biolayer Interferometry

To determine affinity of the humanized variants of chIgG1-ROR2-A for human ROR2 in comparison to that of the rabbit-human chimeric version, a BLI set-up similar to that described in Example 2 was used, with the following changes: preconditioning of the AHC sensors was repeated 2 times, the antibody concentration was 1 μg/mL, association measurement was 1500 s, dissociation measurement was 1500 s, and the analyte (ROR2-ECD) was used as analyte in a concentration range of 1.56-100 nM. Data traces were corrected per antibody by subtraction of the reference sensor. Data were analyzed using Data Analysis Software v9.0.0.12 (ForteBio), using the 1:1 model and a global full fit with 1500 s association time and 200 seconds dissociation time.

Table 8 shows the association rate constant kₐ (1/Ms), dissociation rate constant k_{d} (1/s) and equilibrium dissociation constant K_{D} (M) for human ROR2-ECD of rabbit-human chimeric antibody chIgG1-ROR2-A (with the Fc mutations FEAR) and humanized variants of this antibody.

Table 8: Binding affinities of rabbit-human chimeric antibody chIgG1-ROR2-A and humanized variants of this antibody for recombinant human ROR2-ECD (G&P Biosciences) as determined by label-free biolayer interferometry

| | On-rate | Off-rate | K_{D} |
|---|---|---|---|
| Antibody | kₐ (1/Ms) | k_{d} (1/s) | (nM) |
| chIgG1-ROR2-A-FEAR | 7.2E+05 | 7.9E−04 | 1.1 |
| IgG1-ROR2-A-HC1LC1-FEAR | 5.9E+05 | 9.6E−04 | 1.6 |
| IgG1-ROR2-A-HC1LC2-FEAR | 5.9E+05 | 1.1E−03 | 1.9 |
| IgG1-ROR2-A-HC1LC3-FEAR | 5.5E+05 | 9.7E−04 | 1.8 |
| IgG1-ROR2-A-HC1LC4-FEAR | 5.6E+05 | 1.2E−03 | 2.0 |
| IgG1-ROR2-A-HC2LC1-FEAR | 5.1E+05 | 8.5E−04 | 1.7 |
| IgG1-ROR2-A-HC2LC2-FEAR | 5.1E+05 | 8.9E−04 | 1.8 |
| IgG1-ROR2-A-HC2LC3-FEAR | 4.9E+05 | 1.0E−03 | 2.0 |
| IgG1-ROR2-A-HC2LC4-FEAR | 5.1E+05 | 1.1E−03 | 2.2 |
| IgG1-ROR2-A-HC3LC1-FEAR | 5.5E+05 | 8.8E−04 | 1.6 |
| IgG1-ROR2-A-HC3LC2-FEAR | 5.6E+05 | 1.0E−03 | 1.8 |
| IgG1-ROR2-A-HC3LC3-FEAR | 5.5E+05 | 1.0E−03 | 1.8 |
| IgG1-ROR2-A-HC3LC4-FEAR | 5.8E+05 | 8.7E−04 | 1.5 |
| IgG1-ROR2-A-HC4LC1-FEAR | 5.4E+05 | 1.1E−03 | 2.1 |
| IgG1-ROR2-A-HC4LC2-FEAR | 5.9E+05 | 9.5E−04 | 1.6 |
| IgG1-ROR2-A-HC4LC3-FEAR | 6.0E+05 | 6.8E−04 | 1.1 |
| IgG1-ROR2-A-HC4LC4-FEAR | 6.2E+05 | 9.2E−04 | 1.5 |
| IgG1-ROR2-A-HC5LC1-FEAR | 5.4E+05 | 8.8E−04 | 1.6 |
| IgG1-ROR2-A-HC5LC2-FEAR | 5.5E+05 | 9.7E−04 | 1.8 |
| IgG1-ROR2-A-HC5LC3-FEAR | 5.1E+05 | 8.4E−04 | 1.7 |
| IgG1-ROR2-A-HC5LC4-FEAR | 5.5E+05 | 1.1E−03 | 1.9 |
| IgG1-ROR2-A-HC6LC1-FEAR | 5.6E+05 | 1.7E−03 | 3.1 |
| IgG1-ROR2-A-HC6LC2-FEAR | 5.6E+05 | 2.2E−03 | 3.9 |
| IgG1-ROR2-A-HC6LC3-FEAR | 5.6E+05 | 2.3E−03 | 4.0 |
| IgG1-ROR2-A-HC6LC4-FEAR | 5.7E+05 | 2.5E−03 | 4.4 |
| IgG1-ROR2-A-HC7LC1-FEAR | 5.4E+05 | 2.1E−03 | 3.8 |
| IgG1-ROR2-A-HC7LC2-FEAR | 5.4E+05 | 2.5E−03 | 4.7 |
| IgG1-ROR2-A-HC7LC3-FEAR | 5.5E+05 | 2.8E−03 | 5.1 |
| IgG1-ROR2-A-HC7LC4-FEAR | 5.7E+05 | 3.2E−03 | 5.6 |

From these data it can be seen that the variant IgG1-ROR2-HC4LC3 has a binding affinity that is very comparable to the parent antibody chIgG1-ROR2-A.

### Example 7—Binding of Humanized Variants of chIgG1-ROR2-A to ROR2 Expressed on Cervical Cancer Cell Line HeLa

Binding of humanized variants of chIgG1-ROR2-A to ROR2 expressed on human tumor cells was determined by flow cytometry, using the ROR2 expressing cervical adenocarcinoma cell line HeLa.

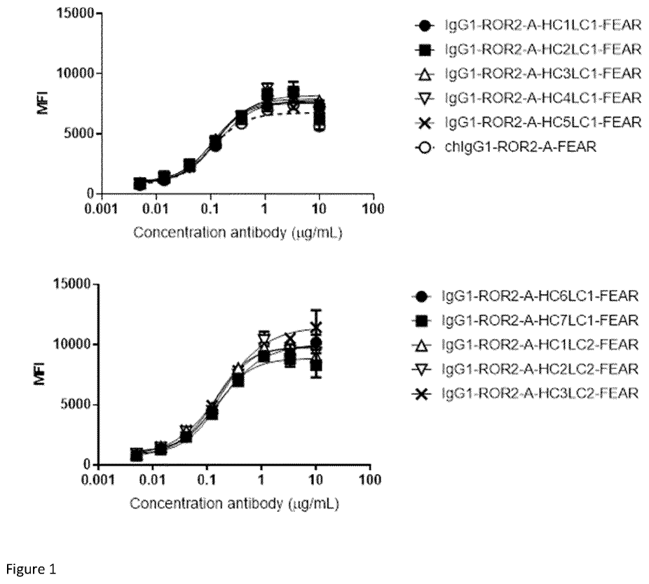

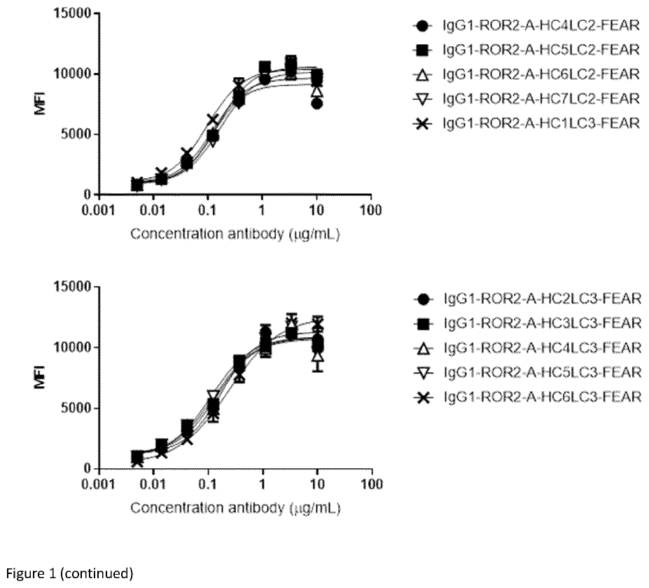

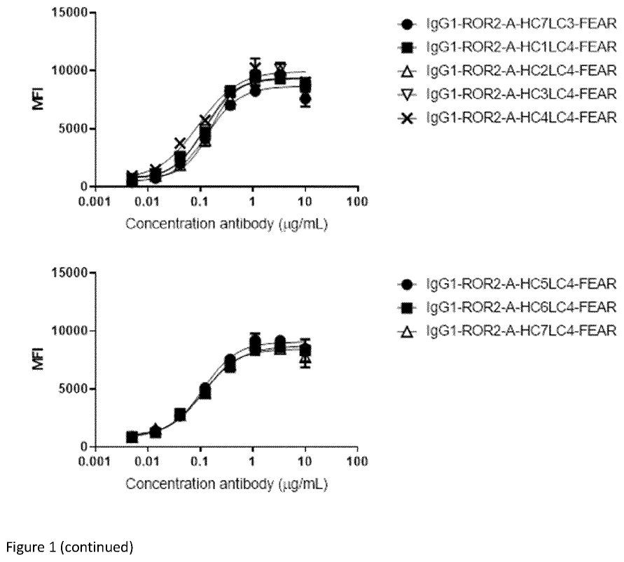

FIG. 1 depicts that rabbit-human chimeric antibody chIgG1-ROR2-A-FEAR and all humanized variants of this antibody showed dose-dependent binding to HeLa cells.

### Example 8—Humanized CD3 Antibodies for the Generation of CD3×ROR2 Bispecific Antibodies

The generation of humanized antibody IgG1-huCD3-H1L1 (of which the variable heavy and light chain region sequences are listed herein in SEQ ID NO: 29 and 30) is described in Example 1 of WO2015/001085. IgG1-huCD3-H1L1 is referred to herein as ‘IgG1-huCD3’. Antibody IgG1-huCD3-H1L1-FEAL is a variant hereof with three amino acid substitutions in the Fc region (L234F, L235E, D265A; FEA), in addition to an amino acid substitution that allows the generation of bispecific antibodies through controlled Fab-arm exchange (F405L), as described herein below. It has been shown that such mutations did not have effect on target binding of the antibodies in which they are introduced (see e.g. WO 2014/108483 and Engelberts et al., 2020, EBioMedicine 52: 102625). Fc regions having the FEA mutations are inert Fc regions, i.e. unable to induce Fc-mediated antibody effector functions through binding of FcγR or C1q.

The generation of humanized antibody IgG1-huCD3-H1L1-H101G (of which the variable heavy chain and light chain region sequences are listed as SEQ ID NO: 32 and 30 herein) is described in Example 2 of WO2017/009442. IgG1-huCD3-H1L1-H101G will be referred to as ‘IgG1-huCD3-H101G’. This variant comprises a substitution H101G (IMGT numbering) in the variable heavy chain region sequence (compare SEQ ID NO. 29 and 32) and has the same light chain as IgG1-huCD3-H1L1. Antibody IgG1-huCD3-H101G-FEAL is a variant hereof with constant region amino acid substitutions L234F, L235E, D265A (FEA) and F405L (Eu numbering).

### Example 9—CD3 Binding Affinity Determination Using Biolayer Interferometry

Binding affinities of IgG1-huCD3-FEAL and IgG1-huCD3-H101G-FEAL were determined as described in Example 7 of WO2017/009442.

In short, binding affinities of selected CD3 antibodies in an IgG1-huCD3-FEAL format for recombinant soluble CD3ε (CD3E27-GSKa) (mature protein of SEQ ID NO:21) were determined using biolayer interferometry on a ForteBio Octet HTX (ForteBio). Anti-human Fc capture biosensors (ForteBio, cat. no. 18-5060) were loaded for 600 s with hIgG (1 μg/mL). After a baseline measurement (200 s), the association (1000 s) and dissociation (2000 s) of CD3E27-GSKa was determined, using a CD3E27-GSKa concentration range of 27.11 μg/mL-0.04 μg/mL (1000 nM-1.4 nM) with three-fold dilution steps (sample diluent, ForteBio, cat. no. 18-5028). For calculations, the theoretical molecular mass of CD3E27-GSKa based on the amino acid sequence was used, i.e. 27.11 kDa. Experiments were carried out while shaking at 1000 rpm and at 30° C. Each antibody was tested in at least two independent experiments. Data was analyzed with ForteBio Data Analysis Software v8.1, using the 1:1 model and a global full fit with 1000 s association time and 100 s dissociation time. Data traces were corrected by subtraction of a reference curve (antibody on biosensor, measurement with sample diluent only), the Y-axis was aligned to the last 10 s of the baseline, and interstep correction as well as Savitzky-Golay filtering was applied. Data traces with a response <0.05 nm was excluded from analysis.

Table 9 shows the association rate constant kₐ (1/Ms), dissociation rate constant k_{d} (1/s) and equilibrium dissociation constant K_{D} (M) for recombinant CD3ε determined by biolayer interferometry. IgG1-huCD3-FEAL showed a relatively high (K_{D}: 15 nM) binding affinity to recombinant CD3ε compared to IgG1-huCD3-H101G-FEAL (K_{D}: 683 nM).

Table 9: Binding affinities of monospecific, bivalent CD3 antibodies to recombinant CD3ε as determined by label-free biolayer interferometry

| | On-rate | Off-rate | K_{D} |
|---|---|---|---|
| Antibody | kₐ (1/Ms) | k_{d} (1/s) | (nM) |
| IgG1-huCD3-FEAL | 2.7E+05 | 4.0E−03 | 15 |
| IgG1-huCD3-H101G-FEAL | 3.0E+04 | 2.0E−02 | 683 |

### Example 10—Generation of Bispecific Antibodies by 2-MEA-Induced Fab-Arm Exchange

Bispecific antibodies were generated in vitro using the DuoBody® platform technology, i.e. 2-MEA-induced Fab-arm exchange as described in WO2011131746 and WO2013060867 (Genmab) and Labrijn et al. (Labrijn et al., PNAS 2013, 110: 5145-50; Gramer et al., MAbs 2013, 5: 962-973). To enable the production of bispecific antibodies by this method, IgG1 molecules carrying specific point mutations in the CH3 domain were generated: in one parental IgG1 antibody the F405L mutation (i.e. the CD3 antibodies in this application), in the other parental IgG1 antibody the K409R mutation (i.e. the humanized IgG1-ROR2 or control, HIV-1 gp120-specific, antibodies in this application). In addition to these mutations, both parental IgG1 antibodies included substitutions L234F, L235E, D265A (FEA).

To generate bispecific antibodies, the two parental antibodies were mixed in equal mass amounts in PBS buffer (Phosphate Buffered Saline; 8.7 mM HPO₄²⁻, 1.8 mM H₂PO₄⁻, 163.9 mM Na⁺, and 140.3 mM Cl⁻, pH 7.4). 2-mercaptoethylamine-HC1 (2-MEA) was added to a final concentration of 75 mM and the reaction mixture was incubated at 31° C. for 5 h. The 2-MEA was removed by dialysis into PBS buffer using 10 kDa molecular-weight cutoff Slide-A-Lyzer carriages (Thermo Fisher Scientific) according to the manufacturer's protocol in order to allow re-oxidation of the inter-chain disulfide bonds and formation of intact bispecific antibodies.

The following ROR2 antibodies, based on rabbit-chimeric antibody chIgG1-ROR2-A or the humanized variant IgG1-ROR2-A-HC4LC3 were used as the parental antibodies to generate the bispecific antibodies in the examples below:

ROR2 Antibodies

chIgG1-ROR2-A-FEAR (having the VH and VL sequences set forth in SEQ ID NO: 2 and SEQ ID NO: 6).

IgG1-ROR2-A-HC4LC3-FEAR (having the VH and VL sequences set forth in SEQ ID NO: 13 and SEQ ID NO: 19).

The annotation IgG1 indicates that full length antibodies of the IgG1 isotype were made, and the FEAR annotation indicates that the heavy chain constant regions contains amino acid substitutions L234F, L235E, D265A and F409R (SEQ ID NO. 34). The light chain constant regions were of the kappa type (SEQ ID NO. 36).

CD3 Antibodies

The following CD3 antibodies were used as the parental antibodies to generate the bispecific antibodies in the examples below:
IgG1-huCD3-FEAL (having the VH and VL sequences set forth in SEQ ID NO: 29 and SEQ ID NO: 30).
IgG1-huCD3-H101G-FEAL (having the VH and VL sequences set forth in SEQ ID NO: 32 and SEQ ID NO: 30).

The annotation IgG1 indicates that full-length antibodies of the IgG1 isotype were made, and the FEAL annotation indicates that the heavy chain constant regions contain amino acid substitutions L234F, L235E, D265A and F405L (SEQ ID NO. 35). The light chain constant regions were of the lambda type (SEQ ID NO. 37).

Bispecific Antibodies

The CD3 and ROR2 antibodies described above were combined to generate bispecific antibodies, having one antigen-binding region capable of binding human CD3 and one antigen-binding region capable of binding human ROR2, providing a bispecific antibodies of the isotype IgG1 which is annotated as bsIgG1.
bsIgG1-huCD3-FEAL×chROR2-A-FEAR (having a rabbit-human chimeric based ROR2 binding arm)
bsIgG1-huCD3-FEAL×ROR2-A-HC4LC3-FEAR (having a humanized ROR2 binding arm)
bsIgG1-huCD3-H101G-FEAL×chROR2-A-FEAR
bsIgG1-huCD3-H101G-FEAL×ROR2-A-HC4LC3-FEAR

In addition, bispecific control antibodies having one antigen-binding region capable of binding human CD3 and one antigen-binding region capable of binding HIV gp120 (derived from antibody b12; Barbas, C. F. et al., 1993. J Mol Biol. 230(3): p. 812-23). As the HIV gp120 protein is not present in any of the assays described here, the Fab-arm binding to HIV gp120-specific antigen-binding region is considered a non-binding control arm.
bsIgG1-huCD3-FEAL×b12-FEAR (the b12 arm has the VH and VL sequences set forth in SEQ ID NO: 47 and SEQ ID NO: 48)
bsIgG1-huCD3-H1010G-FEAL×b12-FEAR

### Example 11—Binding of ROR2 Monospecific Antibody A and CD3×ROR2 Bispecific Antibodies to CHO Cells Expressing Human or Cynomolgus Monkey ROR2, or a Variant of Cynomolgus Monkey ROR2 Carrying a T322M Mutation

First, binding of bispecific CD3×ROR2 antibodies, with either huCD3 or huCD3-H101G as CD3 binding arm, and monospecific ROR2 antibodies to CHO cells expressing human ROR2 (but not human CD3) was determined by flow cytometry essentially as described above, using 3×10⁴ transfected cells/well and an antibody concentration range from 0.00013-10 μg/mL. chIgG1-ROR2-A-FEAR, bsIgG1-huCD3-FEAL×chROR2-A-FEAR, bsIgG1-huCD3-FEAL×ROR2-A-HC4LC3-FEAR, bsIgG1-huCD3-H101G-FEAL×chROR2-A-FEAR and bsIgG1-huCD3-H101G-FEAL×ROR2-A-HC4LC3-FEAR all showed binding in a similar range to CHO cells expressing human ROR2.

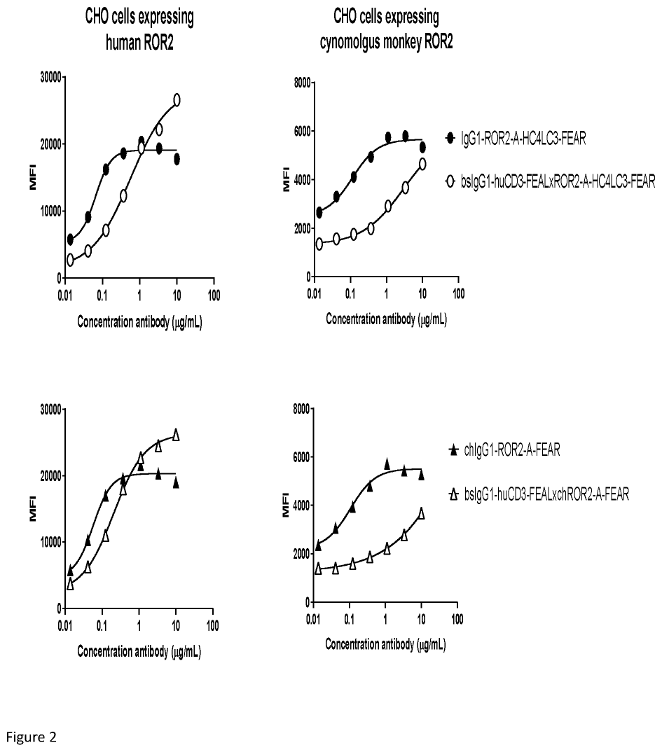

Next, binding of bispecific CD3×ROR2 antibodies and monospecific ROR2 antibodies to CHO cells expressing human or cynomolgus monkey ROR2 was determined, using 5×10⁴ transfected cells/well and an antibody concentration range from 0.01-10 μg/mL. FIG. 2 shows that bsIgG1-huCD3-FEAL×chROR2-A-FEAR, bsIgG1-huCD3-FEAL×ROR2-A-HC4LC3-FEAR, chIgG1-ROR2-A-FEAR and IgG1-ROR2-A-HC4LC3-FEAR all bound to human ROR2 expressed in CHO. chIgG1-ROR2-A-FEAR and IgG1-ROR2-A-HC4LC3-FEAR also bound to cynomolgus monkey ROR2 expressed on CHO cells, but binding of the bispecific bsIgG1-huCD3-FEAL×chROR2-A-FEAR and bsIgG1-huCD3-FEAL×ROR2-A-HC4LC3-FEAR was diminished. In conclusion, whereas monoclonal bivalent ROR2 antibodies efficiently bind human and cynomolgus monkey ROR2, bispecific antibodies containing one ROR2 specific binding domain show reduced binding to cynomolgus monkey but not human ROR2.

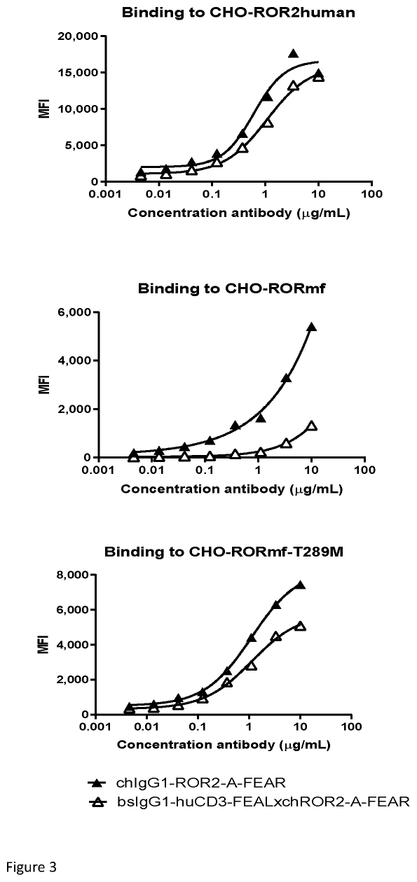

As shown above, the binding domain of chIgG1-ROR2-A involves the kringle domain. The kringle domain sequence of human and cynomolgus monkey ROR2 differs in one amino acid at position 322: T322 in cynomolgus monkey and M322 in human ROR2. The binding of chIgG1-ROR2-A-FEAR and bsIgG1-huCD3-FEAL×chROR2-A-FEAR to CHO cells expressing human ROR2 (SEQ ID NO: 1), cynomolgus monkey ROR2 (ROR2mf, SEQ ID NO: 39) or RORmf-T322M (SEQ ID NO: 41) was determined by flow-cytometry. FIG. 3 shows that chIgG1-ROR2-A-FEAR and bsIgG1-huCD3-FEAL×chROR2-A-FEAR both bind to human ROR2, while binding of bsIgG1-huCD3-FEAL×chROR2-A-FEAR to ROR2mf was diminished compared to chIgG1-ROR2-A-FEAR binding. Binding of bsIgG1-huCD3-FEAL×chROR2-A-FEAR was restored to the range of binding of chIgG1-ROR2-A-FEAR for CHO cells expressing ROR2mf-T322M. This indicates that residue 322 of the mature human ROR2 protein is involved in binding of chIgG1-ROR2-A-FEAR and bsIgG1-huCD3-FEAL×chROR2-A-FEAR.

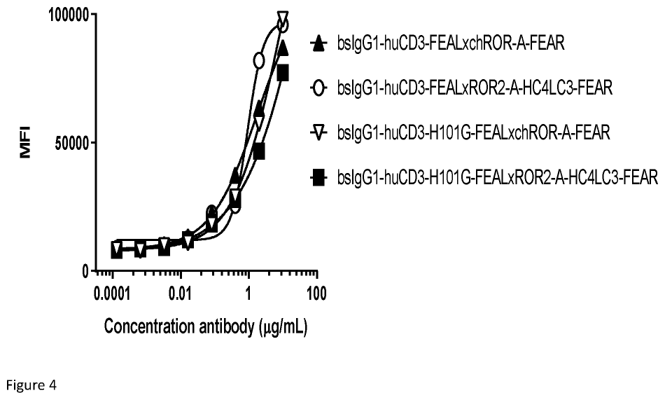

An additional experiment showed that bsIgG1-huCD3-FEAL×chROR2-A-FEAR, bsIgG1-huCD3-FEAL×ROR2-A-HC4LC3-FEAR, bsIgG1-huCD3-H101G-FEAL×chROR2-A-FEAR and bsIgG1-huCD3-H101G-FEAL×ROR2-A-HC4LC3-FEAR all showed comparable binding to RORmf-T322M (FIG. 4).

Thus, based on the binding analysis studies above, results obtained using the chimeric variant of the antibody ROR2-A (chIgG1-ROR2-A or chIgG1-ROR2-A-FEAR) or bispecific antibodies derived of the chimeric variant (bsIgG1-huCD3-FEAL×chROR2-A-FEAR or bsIgG1-huCD3-H101G-FEAL×chROR2-A-FEAR) also apply to the humanized variant of this antibody (IgG1-ROR2-A-HC4LC3-FEAR) or bispecific antibodies derived from the humanized variant (bsIgG1-huCD3-FEAL×ROR2-A-HC4LC3-FEAR or bsIgG1-huCD3-H101G-FEAL×ROR2-A-HC4LC3-FEAR). Accordingly, amino acid residue M322 of the kringle domain of the mature human ROR2 protein (SEQ ID NO: 1) is involved in binding of these ROR2 binding antibodies.

### Example 12—Binding of bsIgG1-huCD3-H101G-FEAL×chROR2-A-FEAR to ROR2-Expressing Human Tumor Cell Lines

Binding of bsIgG1-huCD3-H101G-FEAL×chROR2-A-FEAR to the ROR2-expressing human tumor cell lines HeLa, LCLC103-H (large-cell lung cancer; DSMZ, cat. no. ACC-384), NCI-H1650 (lung adenocarcinoma; ATCC, cat. no. CRL-5883), 786-0 (renal cell adenocarcinoma; ATCC, cat no. CRL-1932), NCI-H23 (lung adenocarcinoma; ATCC, cat. no. CRL-5800) and ZR-75-1 (breast ductal carcinoma; ATCC, cat. no. CRL-1500) was determined in vitro. ROR2 expression levels were determined by quantitative flow cytometry (Human IgG calibrator, BioCytex) according to the manufacturer's instructions, using bsIgG1-huCD3-H101G-FEAL×chROR2-A-FEAR to detect ROR2. Binding was analyzed by flow cytometry as described above, using 3×10⁴ tumor cells/well and antibody concentrations ranging from 0.014-30 μg/mL. bsIgG1-huCD3-H1010G-FEAL×b12-FEAR, that is able to bind CD3 but not ROR2, was used as negative control antibody.

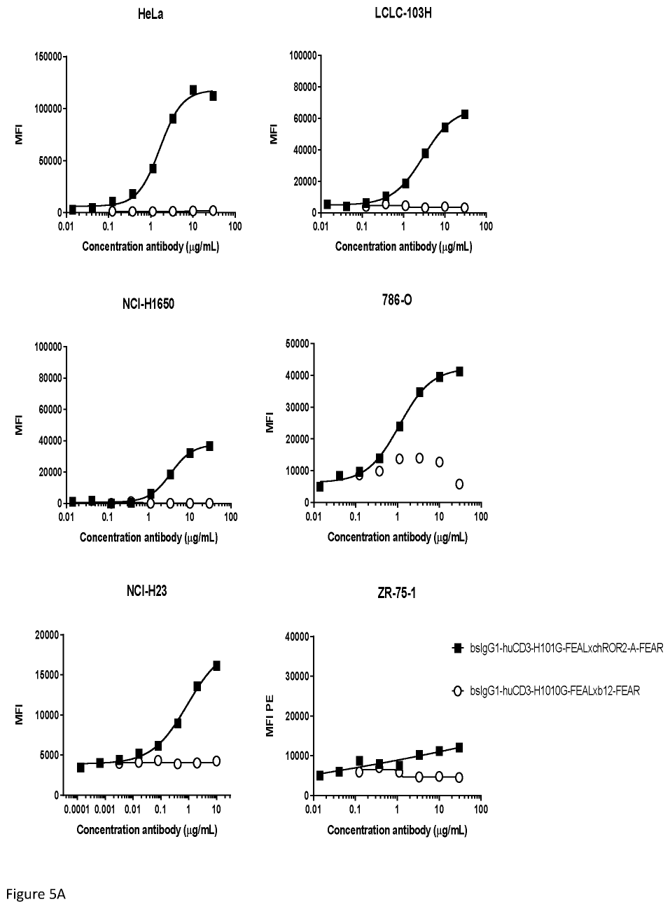

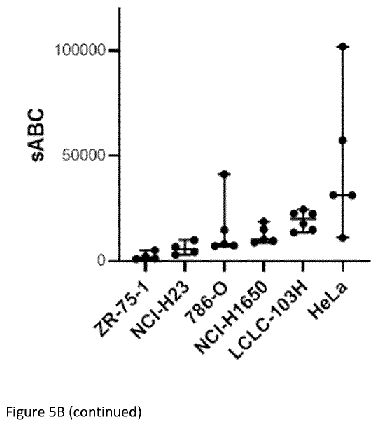

FIG. 5 shows that bsIgG1-huCD3-H101G-FEAL×chROR2-A-FEAR binds dose-dependently to the tumor cell lines, with highest maximum binding as determined by MFI (FIG. 5A) corresponding to highest target expression determined by semi-quantitative flow cytometry (FIG. 5B).

### Example 13—Induction of T Cell Mediated Cytotoxicity In Vitro by CD3×ROR2 Bispecific Antibodies in Co-Cultures of ROR2 Positive Tumor Cells (HeLa) and Human Healthy Donor T Cells at Different Effector:Target Ratio's

To determine the efficiency of T cell-mediated tumor cell kill in the presence of bispecific CD3×ROR2 antibodies, bsIgG1-huCD3-FEAL×chROR2-A-FEAR and bsIgG1-huCD3-H101G-FEAL×chROR2-A-FEAR, an in vitro cytotoxicity assay was performed using ROR2-positive HeLa cells as target cells (T) and purified T cells as effector cells (E), with varying effector to target cell (E:T) ratios.

T cells were obtained from healthy human donor buffy coats (Sanquin, Amsterdam, The Netherlands) and purified using the RosetteSep™ human T cell enrichment cocktail (Stemcell Technologies, France, cat. no. 15061) according to the manufacturer's instructions. HeLa cells (16,000 cells/well) were seeded into flat bottom 96-well plates (Greiner-bio-one, The Netherlands, cat. no. 655180) and left to adhere for 4 hours at 37° C. T cells were added to tumor cells at an E:T ratio of 1:1, 2:1, 4:1, 8:1, 12:1 or 16:1. Serial dilutions of bsIgG1-huCD3-FEAL×chROR2-A-FEAR, bsIgG1-huCD3-H101G-FEAL×chROR2-A-FEAR or bsIgG1-huCD3-FEAL×b12-FEAR were added (final concentration ranging from 10,000 to 0.0005 ng/mL; 5-fold dilutions) and plates were incubated for 72 hours at 37° C. Plates were washed 3 times with PBS, and adherent cells were incubated with 150 μl/well of 10% alamarBlue® solution (Invitrogen, cat. no. DAL1100) for 4 hours at 37° C. to determine viability of the tumor cells. As a positive control for cytotoxicity, cells were incubated with 16 μg/mL phenylarsine oxide (PAO; Sigma-Aldrich, cat. no. P3075; dissolved in dimethyl sulfoxide [DMSO; Sigma-Adrich, cat. no. D2438]). AlamarBlue fluorescence, as a measure of metabolic activity of the tumor cell cultures and thus of viable tumor cells, was measured at 615 nm (OD615) on an EnVision plate reader (PerkinElmer). The absorbance of PAO-treated tumor cell samples was set as 0% viability and the absorbance of untreated tumor cell samples was set as 100% viability. The ‘percentage viable cells’ was calculated as follows:

% viable cells=([absorbance sample−absorbance PAO-treated target cells]/[absorbance untreated target cells−absorbance PAO-treated target cells])×100.

Dose-response curves and IC50 values were generated using non-linear regression analysis (sigmoidal dose-response with variable slope) using GraphPad Prism V7.02 software (GraphPad Software, San Diego, CA, USA).

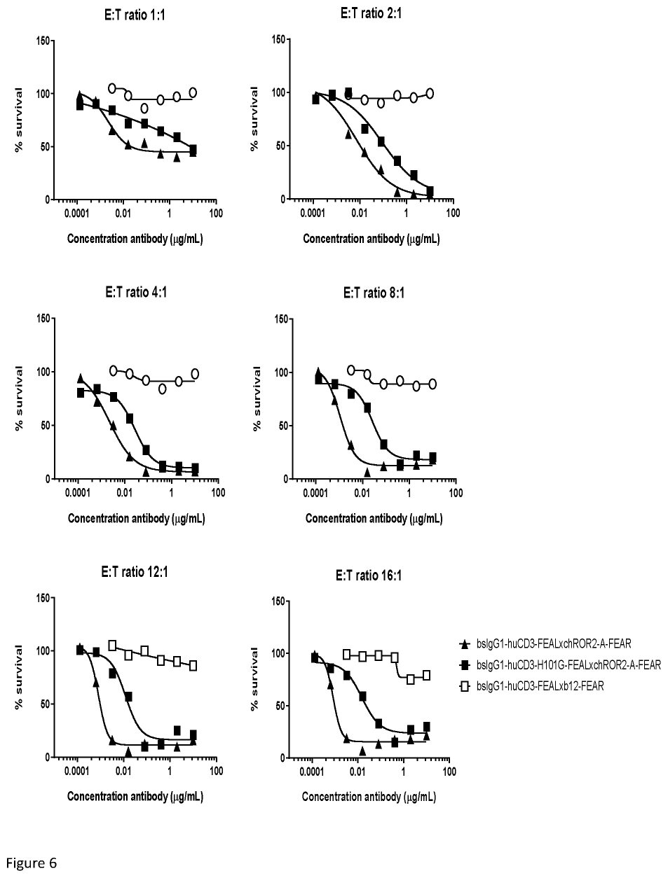

FIG. 6 shows that dose-dependent T cell mediated cytotoxicity was observed at all E:T ratio's, with maximal tumor cell killing (less than 10% viable tumor cells) observed for E:T ratios above 2:1. While maximum cytotoxic activity (<10% viable tumor cells) was achieved for both bsAb variants, this occurred at lower concentrations for bsIgG1-huCD3-FEAL×chROR2-A-FEAR in comparison with bsIgG1-huCD3-H101G-FEAL×chROR2-A-FEAR. Bispecific control antibody bsIgG1-huCD3-FEAL×b12-FEAR, that binds CD3 but not ROR2, did not induce T cell mediated cytotoxicity. Also, no T cell mediated cytotoxicity was observed using a ROR2 negative cell line (HT-29; colorectal adenocarcinoma; ATCC, cat. no. HTB-38) as target cell (data not shown). These data indicate that bsIgG1-huCD3-FEAL×chROR2-A-FEAR and bsIgG1-huCD3-H101G-FEAL×chROR2-A-FEAR are capable of inducing T cell-mediated kill of ROR2 expressing Hela cells. While bsIgG1-huCD3-FEAL×chROR2-A-FEAR shows an effect at lower concentrations than bsIgG1-huCD3-H101G-FEAL×chROR2-A-FEAR, both reach the same maximum cytotoxic effect at a given E:T ratio.

### Example 14—Induction of Cytotoxicity In Vitro in Various ROR2 Positive Tumor Cell Lines by CD3×ROR2 Bispecific Antibodies in the Presence of Human Healthy Donor T Cells

The T cell-mediated kill of bispecific antibodies bsIgG1-huCD3-FEAL×chROR2-A-FEAR and bsIgG1-huCD3-H101G-FEAL×chROR2-A-FEAR of various ROR2 expressing tumor cell lines was determined in an in vitro cytotoxicity assay as described above, using an E:T ratio of 8:1. The following cell lines were used: HeLa, LCLC103-H, NCI-H1650, 786-0, NCI-H23 and ZR-75-1 (see above for further information on the tumor cell lines).

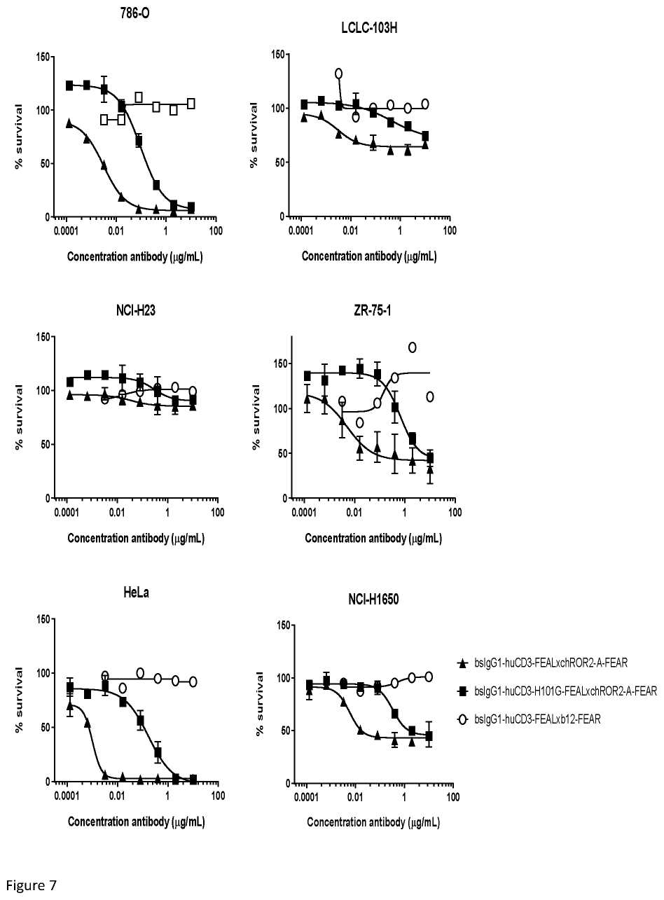

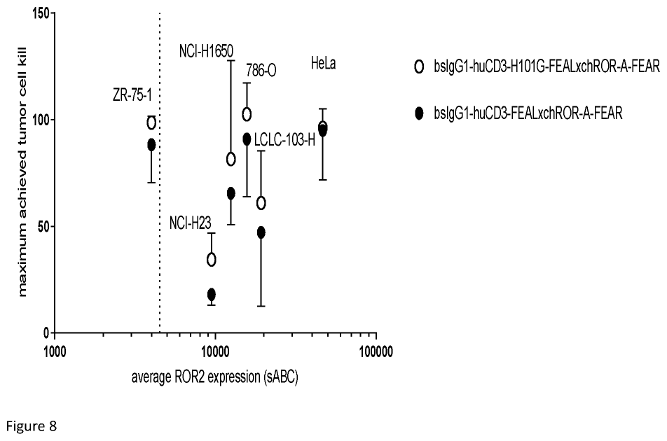

FIG. 7 shows that both bsIgG1-huCD3-FEAL×chROR2-A-FEAR and bsIgG1-huCD3-H101G-FEAL×chROR2-A-FEAR induced dose-dependent T cell mediated cytotoxicity of HeLa, LCLC103-H, NCI-H1650, 786-0, NCI-H23 and ZR-75-1 cells in vitro. Tumor cell kill occurred at lower concentrations for bsIgG1-huCD3-FEAL×chROR2-A-FEAR in comparison with bsIgG1-huCD3-H101G-FEAL×chROR2-A-FEAR (Table 10) indicating that bsIgG1-huCD3-FEAL×chROR2-A-FEAR is more potent in tumor cell kill than bsIgG1-huCD3-H101G-FEAL×chROR2-A-FEAR. Maximal tumor cell kill was comparable between the bsIgG1-huCD3-FEAL×chROR2-A-FEAR and bsIgG1-huCD3-H101G-FEAL×chROR2-A-FEAR. No correlation between the extent of T cell mediated cytotoxicity and the ROR2 expression level per cell line (FIG. 8) was observed on the panel of cell lines as studied with T cells from different donors.

TABLE 10
Induction of cytotoxicity in vitro in various tumor cell lines by CD3 × ROR2
bispecific antibodies in the presence of human healthy donor T cells: IC50 values
are the geomean of IC50 of evaluable dose-response curves (number of donors indicated).

| | bsIgG1-huCD3-H101G-FEAL × chROR2-A-FEAR | | bsIgG1-huCD3-FEAL × chROR2-A-FEAR | |
|---|---|---|---|---|
| Cell line | IC50 (μg/mL) | n donors | IC50 (μg/mL) | n donors |
| ZR-75-1 | 0.424 | 2 | 0.003 | 2 |
| (breast cancer) | | | | |
| NCI-H23 | 0.580 | 4 | 0.013 | 3 |
| (lung cancer) | | | | |
| 786-O | 0.088 | 4 | 0.003 | 4 |
| (renal cancer) | | | | |
| NCI-H1650 | 0.693 | 3 | 0.006 | 3 |
| (lung cancer) | | | | |
| LCLC-103H | 0.511 | 5 | 0.011 | 7 |
| (large cell lung | | | | |
| cancer) | | | | |
| Hela | 0.424 | 6 | 0.002 | 6 |
| (cervical cancer) | | | | |

### Example 15—Induction of Cytokine Production In Vitro by CD3×ROR2 Bispecific Antibodies in the Presence of ROR2-Positive Tumor Cells

This experiment was performed to demonstrate that the CD3×ROR2 bispecific antibodies of the invention activate T cells and induce cytokine production in the presence of ROR2-expressing target cells.

From the wells incubated for T cell-mediated cytotoxicity by CD3×ROR2 bispecifics of HeLa and 786-0 cells as described above, 150 μL supernatants was transferred to U-bottom 96 Well culture plates (CellStar, cat. no. 650180) to determine cytokine levels. The plates were centrifuged (300×g) for 3 min at 4° C. to remove cells, after which 75 μL of supernatant was transferred to a new plate for cytokine production measurement by Mesoscale Discovery U-plex multiplex ELISA (MeSo Scale Discovery, USA, cat. no. K15049K).

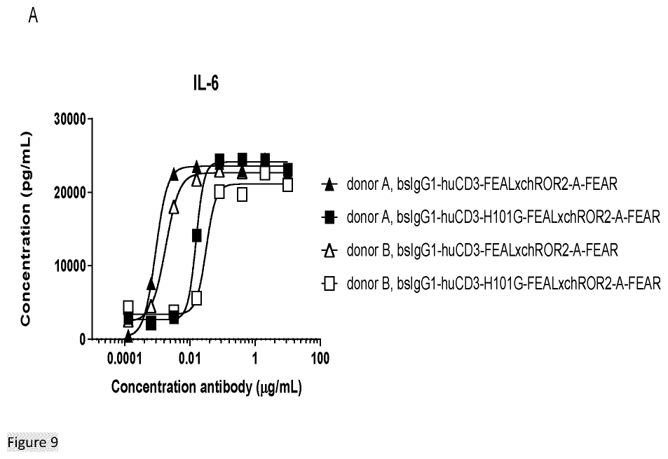

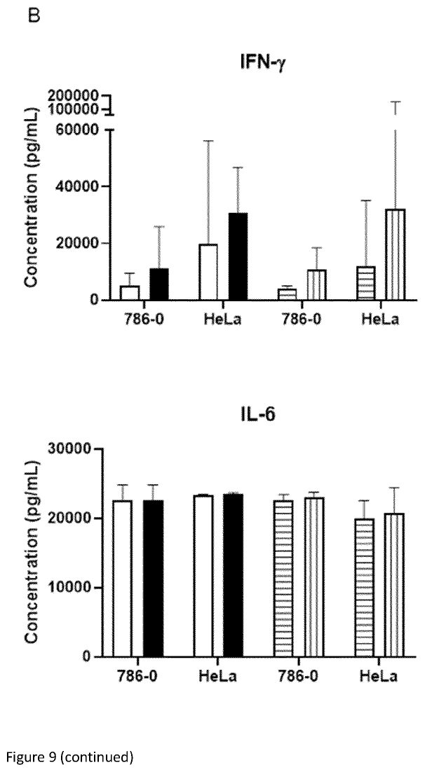

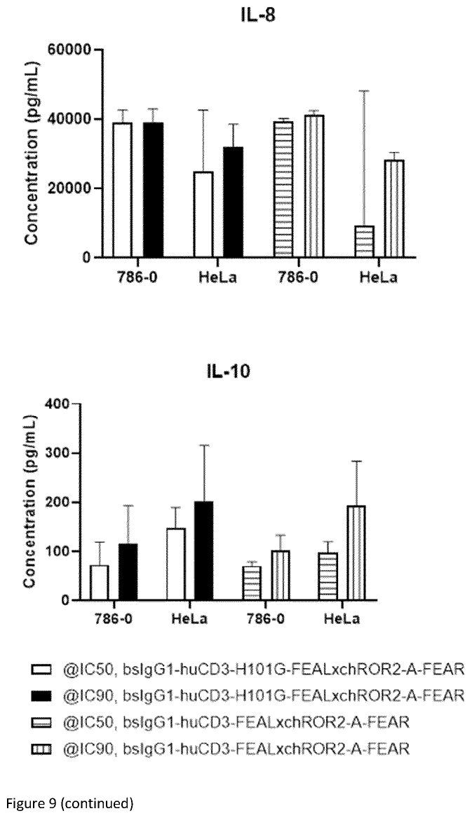

Of the 10 cytokines analyzed, significant increases were primarily observed for IFN-gamma, IL-6, IL-8 and IL-10 (>100 μg/ml). IL-4, IL-13, IL-1beta, IL-2, IL-12p70 and TNFalpha levels were generally below 100 μg/ml. FIG. 9A shows the levels of IL-6 in the supernatant of T cell-tumor cell co-cultures with increasing concentrations of antibodies bsIgG1-huCD3-FEAL×chROR2-A-FEAR or bsIgG1-huCD3-H101G-FEAL×chROR2-A-FEAR, using T cells from 2 donors and 786-0 cells as tumor cells. FIG. 9B shows the levels of IFN-gamma, IL-6, IL-8 and IL-10 at antibody concentrations that induced T cell mediated cytotoxicity in 50% and 90% tumor cells (IC50 and IC90), using HeLa or 786-0 cells as tumor cells. Cytokine production levels varied per donor and per target tumor cell line. At concentrations associated with 50% or 90% cytotoxicity cytokine levels were comparable for the bsIgG1-huCD3-FEAL×chROR2-A-FEAR or bsIgG1-huCD3-H101G-FEAL×chROR2-A-FEAR. The data indicate that bsIgG1-huCD3-FEAL×chROR2-A-FEAR and bsIgG1-huCD3-H101G-FEAL×chROR2-A-FEAR both induce cytokine production in vitro in the presence of ROR2-positive tumor cells.

### Example 16—Capacity of CD3×ROR2 Bispecific Antibodies to Induce Cytotoxic Activity and Activation of Cynomolgus Monkey T Cells In Vitro in the Presence of ROR2 Positive Tumor Cells HeLa Cells

To determine the efficiency of tumor cell kill by peripheral blood mononuclear cells (PBMCs) from cynomolgus monkeys in the presence of bispecific CD3×ROR2 antibodies, bsIgG1-huCD3-FEAL×ROR2-A-HC4LC3-FEAR and bsIgG1-huCD3-H101G-FEAL×ROR2-A-HC4LC3-FEAR, an in vitro cytotoxicity assay was performed essentially as described above, using HeLa cells as target cells, at an PBMC:target cell ratio of 8:1. Cynomolgus monkey PBMCs were obtained from Zen-Bio (USA). Flow cytometric analysis if the PMBCs showed that approximately 65% of cells are CD3+(T) cells). The experiment was designed to confirm that CD3×ROR2 bispecific antibodies were able to activate and engage cynomolgus monkey T cells as effector cells, and thus that cynomolgus monkeys may be considered a relevant species to assess the (non-clinical) safety of the bispecific antibodies of the invention.

To measure T cell activation, 150 μL supernatant was transferred to 96-well plates after the 72 h incubation time and centrifuged. Cells were stained for T cell markers CD3 (1:100; Miltenyi Biotech, clone 10D12, conjugated to APC; cat. no. 130-091-998), CD4 (1:50; eBioscience, clone OKT4, conjugated to APC-Cy7; cat. no. 47-0048-42), CD8 (1:100; Biolegend, clone RPA-T8, conjugated to AF700; cat. no. 301028) and T cell activation markers CD69 (1:50; BD Biosciences, clone FN50, conjugated to FITC; cat. no. 555530), CD25 (1:100; eBioscience, clone BC96, conjugated to PE-Cy7: cat. no 25-0259-42) and CD279/PD1 (1:50; Biolegend, clone EH12.2H7, conjugated to BV605: cat. no. 340560). Single stained samples with Ultracomp beads (5 μL; Invitrogen, cat. no. 01-2222-42) were included and used for compensation adjustments of the flow cytometer. After 30 min of incubation at 4° C., plates were washed three times with PBS/0.1% BSA/0.02% azide (staining buffer). Cells were resuspended in 80 μL staining buffer and analyzed using a FACS Fortessa (BD Biosciences). Data were processed using FlowJo (BD Biosciences).

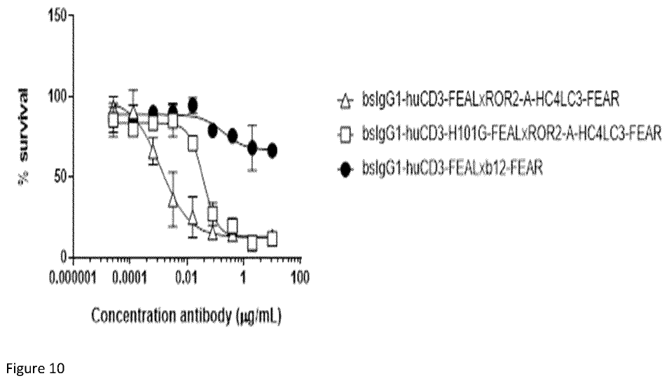

FIG. 10 shows that bsIgG1-huCD3-FEAL×ROR2-A-HC4LC3-FEAR and bsIgG1-huCD3-H101G-FEAL×ROR2-A-HC4LC3-FEAR both induced dose-dependent, cynomolgus monkey PBMC induced kill of tumor cells expressing human ROR2, with killing occurring at lower concentrations for bsIgG1-huCD3-FEAL×ROR2-A-HC4LC3-FEAR in comparison with bsIgG1-huCD3-H101G-FEAL×ROR2-A-HC4LC3-FEAR.

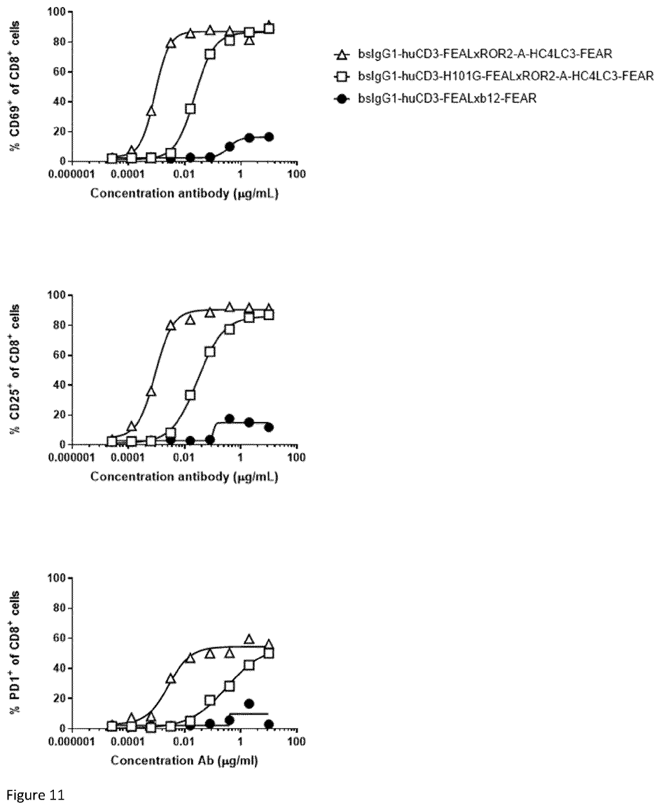

FIG. 11 shows the activation of T cells within the cynomolgus monkey PMBC population in the presence of bsIgG1-huCD3-FEAL×ROR2-A-HC4LC3-FEAR or bsIgG1-huCD3-H101G-FEAL×ROR2-A-HC4LC3-FEAR and HeLa cells, as defined by the expression of activation markers CD69, CD25 and PD-1 on CD8+ T cells (determined by flow cytometry). Approximately 80% (at the highest antibody concentration) of CD8+ T cells became activated and expressed CD69 and CD25 in the presence of either bsIgG1-huCD3-FEAL×ROR2-A-HC4LC3-FEAR or bsIgG1-huCD3-H101G-FEAL×ROR2-A-HC4LC3-FEAR, and approximately 40% of CD8+ T cells expressed PD-1. T cell activation induced by bsIgG1-huCD3-FEAL×ROR2-A-HC4LC3-FEAR occurred at lower concentrations than that induced by bsIgG1-huCD3-H101G-FEAL×ROR2-A-HC4LC3-FEAR. This indicates that bsIgG1-huCD3-FEAL×ROR2-A-HC4LC3-FEAR and bsIgG1-huCD3-H101G-FEAL×ROR2-A-HC4LC3-FEAR are able to engage cynomolgus monkey T cells as effector cells and activate T cells.

### Example 17—ROR2 Expression in Various Human Cancer Indications

ROR2 mRNA levels were extracted from the Omicsoft TCGA database and visualized using Oncoland software (Qiagen, USA).

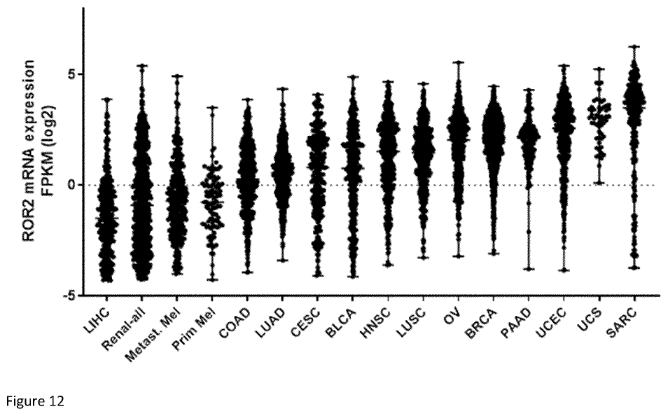

FIG. 12 shows ROR2 mRNA expression levels in a selection of primary solid tumors, ranked according to median expression. mRNA expression varied within each indication, with highest median expression found in sarcoma, uterine, pancreatic, breast and ovarian cancers and lung squamous cell carcinoma.

Protein expression of ROR2 in fibrosarcoma, gastro intestinal stromal tumor (GIST), leiomyosarcoma, rhabdomyosarcoma, liposarcoma, ovarian adenocarcinoma (serous papillary), endometrioid carcinoma, lung squamous cell carcinoma, lung Adenocarcinoma, pancreas cancer, clear cell carcinoma, transitional cell carcinoma and colon adenocarcinoma was analyzed by immunohistochemistry (IHC) on Leica Bond RX with Leica Bond reagents on tissue microarrays (TMA; purchased from BioMax). Prior to staining, freshly cut TMA sections (5 μm) were deparaffinized and incubated with target retrieval solution ER2. ROR2 IHC was performed using a mouse anti-ROR2 antibody (clone ROR2 2535-2835, QED Bioscience, cat. no. 34045) at a final concentration of 10 μg/mL. Subsequently, sections were washed and incubated with goat anti-mouse-IgG-HRP. HRP was visualized with DAB refine substrate chromogen system. Hematoxylin was used to detect nucleated cells. Stained TMA sections were digitized at 20× magnification on an AxioScan slide scanner (Zeiss).

ROR2 staining intensity and the percentage ROR2 positive cells in the tumor was determined and quantified by a certified pathologist. Staining intensity was scored as negative (0), weak (1), moderate (2) or strong (3) and the percentage cells in range of 0-100% with increments of 10%. From the staining intensity and percentage positive cells, the histologic score (H-score) was determined according to:

H-score=(0×[% cells with intensity of 0]+1×[% cells with intensity 1+]+2×[% cells with intensity 2+]+3×[% cells with intensity 3+])

Table 11 shows ROR2 protein expression (prevalence and H-score) determined by IHC analysis of BioMax TMAs. Per indication the ROR2 expression varied. The highest prevalence and ROR2 H scores were found in sarcomas, GIST, and ovarian and endometrioid cancers.

TABLE 11
ROR2 protein expression (prevalence and H-score)
determined by IHC analysis of BioMax TMAs.

| | ROR2 prevalence | |
|---|---|---|
| | (% of ROR2 | |
| | positive tumor | |
| | biopsies per | Mean ROR2 |
| Tumor type | indication) | H score |
| Fibrosarcoma (n = 20) | 100% | 97 |
| GIST (n = 79) | 97% | 147 |
| Leiomyosarcoma (n = 92) | 93% | 86 |
| Rhabdomyosarcoma (n = 19) | 79% | 65 |
| Liposarcoma (n = 17) | 53% | 16 |
| Ovarian AC Serous papillary (n = 28) | 67% | 11 |
| Endometrioid carcinoma (n = 59) | 64% | 32 |
| Lung Squamous cell carcinoma (n = 81) | 38% | 15 |
| Lung Adenocarcinoma (n = 69) | 25% | 3 |
| Pancreas (n = 85) | 30% | 6 |
| Clear cell carcinoma (n = 74) | 15% | 4 |
| Transitional cell carcinoma (n = 17) | 29% | 11 |
| Colon AC (n = 58) | 26% | 5 |

1. An antibody comprising at least one antigen-binding region capable of binding to human ROR2 wherein said antibody comprises a heavy chain variable (VH) region CDR1, CDR2, and CDR3 having the sequences as set forth in SEQ ID NOs: 3, 4, and 5, respectively, and a light chain variable (VL) region CDR1, CDR2, and CDR3 having the sequences as set forth in SEQ ID NO:7, 8 and 9, respectively.

2. The antibody of claim 1 wherein said antibody comprises two antigen-binding regions capable of binding to human ROR2 wherein said antibody comprises the heavy chain variable (VH) region CDR1, CDR2, and CDR3 having the sequences as set forth in SEQ ID NOs: 3, 4, and 5, respectively, and the light chain variable (VL) region CDR1, CDR2, and CDR3 having the sequences as set forth in SEQ ID NO:7, 8 and 9 respectively.

3. The antibody of any one of claims 1 or 2 wherein said antibody is humanized from an antibody which comprises a VH region having the sequence set forth in SEQ ID NO: 2 and/or a VL region having the sequence set forth in SEQ ID NO: 6.

4. The antibody of any one of the above claims wherein said antibody comprises a VH region having a sequence selected from the group comprising:
a. a VH region as set forth in SEQ ID NO:10 (HC1);
b. a VH region as set forth in SEQ ID NO:11 (HC2);
c. a VH region as set forth in SEQ ID NO:12 (HC3);
d. a VH region as set forth in SEQ ID NO:13 (HC4);
e. a VH region as set forth in SEQ ID NO:14 (HC5);
f. a VH region as set forth in SEQ ID NO:15 (HC6);
g. a VH region as set forth in SEQ ID NO:16 (HC7) or
h. a VH region having at least 90% sequence identity to any one of the sequences of SEQ ID NOs 10, 11, 12, 13, 14, 15 or 16

5. The antibody of any one of the above claims wherein said antibody comprises a VH region having the sequence set forth in SEQ ID NO:13.

6. The antibody of any one of the preceding claims wherein said antibody comprises a VL region having a sequence selected from the group comprising:
a. a VL region as set forth in SEQ ID NO:17 (LC1);
b. a VL region as set forth in SEQ ID NO:18 (LC2);
c. a VL region as set forth in SEQ ID NO:19 (LC3);
d. a VL region as set forth in SEQ ID NO:20 (LC4) or
e. a VL region having at least 90% sequence identity to any one of the sequences of SEQ ID NOs 17, 18, 19 or 20.

7. The antibody of any one of the preceding claims wherein said antibody comprises a VL region having the sequence set forth in SEQ ID NO:19.

8. The antibody of any one of the preceding claims wherein said antibody comprises the VH and VL regions having the sequences selected from the group comprising:
a. The VH region having the sequence of SEQ ID NO. 10 and the VL region having the sequence of SEQ ID NO. 17;
b. The VH region having the sequence of SEQ ID NO. 10 and the VL region having the sequence of SEQ ID NO. 18;
c. The VH region having the sequence of SEQ ID NO. 10 and the VL region having the sequence of SEQ ID NO. 19;
d. The VH region having the sequence of SEQ ID NO. 10 and the VL region having the sequence of SEQ ID NO. 20;
e. The VH region having the sequence of SEQ ID NO. 11 and the VL region having the sequence of SEQ ID NO. 17;
f. The VH region having the sequence of SEQ ID NO. 11 and the VL region having the sequence of SEQ ID NO. 18;
g. The VH region having the sequence of SEQ ID NO. 11 and the VL region having the sequence of SEQ ID NO. 19;
h. The VH region having the sequence of SEQ ID NO. 11 and the VL region having the sequence of SEQ ID NO. 20;
i. The VH region having the sequence of SEQ ID NO. 12 and the VL region having the sequence of SEQ ID NO. 17;
j. The VH region having the sequence of SEQ ID NO. 12 and the VL region having the sequence of SEQ ID NO. 18;
k. The VH region having the sequence of SEQ ID NO. 12 and the VL region having the sequence of SEQ ID NO. 19;
l. The VH region having the sequence of SEQ ID NO. 12 and the VL region having the sequence of SEQ ID NO. 20;
m. The VH region having the sequence of SEQ ID NO. 13 and the VL region having the sequence of SEQ ID NO. 17;
n. The VH region having the sequence of SEQ ID NO. 13 and the VL region having the sequence of SEQ ID NO. 18;
o. The VH region having the sequence of SEQ ID NO. 13 and the VL region having the sequence of SEQ ID NO. 19;
p. The VH region having the sequence of SEQ ID NO. 13 and the VL region having the sequence of SEQ ID NO. 20;
q. The VH region having the sequence of SEQ ID NO. 14 and the VL region having the sequence of SEQ ID NO. 17;
r. The VH region having the sequence of SEQ ID NO. 14 and the VL region having the sequence of SEQ ID NO. 18;
s. The VH region having the sequence of SEQ ID NO. 14 and the VL region having the sequence of SEQ ID NO. 19;
t. The VH region having the sequence of SEQ ID NO. 14 and the VL region having the sequence of SEQ ID NO. 20;
u. The VH region having the sequence of SEQ ID NO. 15 and the VL region having the sequence of SEQ ID NO. 17;
v. The VH region having the sequence of SEQ ID NO. 15 and the VL region having the sequence of SEQ ID NO. 18;
w. The VH region having the sequence of SEQ ID NO. 15 and the VL region having the sequence of SEQ ID NO. 19;
x. The VH region having the sequence of SEQ ID NO. 15 and the VL region having the sequence of SEQ ID NO. 20;
y. The VH region having the sequence of SEQ ID NO. 16 and the VL region having the sequence of SEQ ID NO. 17;
z. The VH region having the sequence of SEQ ID NO. 16 and the VL region having the sequence of SEQ ID NO. 18;
aa. The VH region having the sequence of SEQ ID NO. 16 and the VL region having the sequence of SEQ ID NO. 19; and
bb. The VH region having the sequence of SEQ ID NO. 16 and the VL region having the sequence of SEQ ID NO. 20.

9. The antibody according to any one of the preceding claims wherein said antibody comprises the VH and VL regions having the sequences of SEQ ID Nos 13 and 19.

10. The antibody according to any one of the preceding claims wherein the VH and VL regions are humanized.

11. The antibody according to any one of the preceding claims wherein the heavy chain constant region is human IgG1.

12. The antibody according to any one of the preceding claims wherein the light chain constant region is human kappa.

13. The antibody according to any of the preceding claims, wherein the antibody is a full-length antibody, such as a full length IgG1 antibody.

14. The antibody according to any one of the preceding claims, which is a monovalent antibody.

15. The antibody according to any one of the preceding claims, which is a bivalent antibody.

16. The antibody according to any one of the preceding claims, which is a monospecific antibody.

17. The antibody according to any one of the preceding claims, which is a bispecific antibody.

18. The antibody according to any one of the preceding claims, wherein said human ROR2 is human ROR2 of SEQ ID NO. 1.

19. The antibody according to any one of the preceding claims wherein the antibody is able to bind to the Kringle domain of human ROR2.

20. The antibody according to any one of the preceding claims, wherein said antibody binds to an epitope or antibody binding region on human ROR2 that involves the amino acid residue at position 322 of human ROR2, the numbering referring to its position in SEQ ID NO: 1.

21. The antibody according to any one of the preceding claims, said antibody being able to bind human ROR2 extra cellular domain with a binding affinity that corresponds to a KD value of 100 nM or less, such as 50 nM or less, 10 nM or less, 6 nM or less or such as 3 nM or less such as with a binding affinity corresponding to a KD value which is within the range of 100 nM to 0.1 nM, such as within the range of 100 nM to 1 nM, such as 50 nM to 1 nM, such as less than about 2.5 nM or less than about 2.0 nM or less than about 1.5 nM such as about 1.1 nM.

22. The antibody according to claim 21, wherein the binding affinity is determined by biolayer interferometry, optionally as set forth in Example 6 herein.

23. The antibody according to any one of claims 21 and 22, wherein the binding affinity is determined using a biolayer interferometry comprising the steps of:
a. Immobilizing the antibody at an amount of 1 μg/mL for 600 seconds on an anti-human IgG Fc Capture biosensor;
b. Determining association over a time period of 1,500 seconds and dissociation over a time period of 1,500 seconds of ROR2ECDHis using 2-fold dilution series ranging from 100 nM to 1.56 nM.
c. Referencing the data to a buffer control (0 nM).

24. The antibody according to any one of claims 21 to 23, wherein the binding affinity is determined using an antibody as defined in any one of the preceding claims, which is a monospecific, bivalent antibody, such as an antibody which is a full length IgG1.

25. An antibody comprising a first antigen binding region capable of binding human ROR2 according to any one of the preceding claims and comprising a second antigen binding region capable of binding to a different target.

26. The antibody of claim 25 wherein the second antigen binding region is capable of binding to human CD3 such as human CD3ε (epsilon), such as human CD3ε (epsilon) as specified in SEQ ID NO: 21.

27. The antibody according to claim 25 or 26, which is a bispecific antibody.

28. The antibody according to claim 26 or 27, wherein the antigen-binding region that binds to CD3 comprises
a heavy chain variable region (VH) comprising the CDR1, CDR2, and CDR3 sequences of SEQ ID NOs.: 23, 24 and 25, respectively;
and, optionally
a light chain variable region (VL) comprising the CDR1, CDR2, and CDR3 sequences of SEQ ID NO: 27, GTN and 28, respectively.

29. The antibody according to any one of claims 26 to 28, wherein the antigen binding region that binds to CD3 comprises
a. a heavy chain variable region (VH) comprising the sequence of SEQ ID NO: 22, or a sequence having at least 80%, least 90%, at least 95%, at least 97%, or at least 99% amino acid sequence identity to the sequence of SEQ ID NO: 22;
b. and a light chain variable region (VL) comprising the sequence of SEQ ID NO: 26 or a sequence having at least 80%, at least 90%, at least 95%, at least 97%, or at least 99% amino acid sequence identity to the sequence of SEQ ID NO: 26.

30. The antibody according to any one of claims 26 to 29, wherein the antigen binding region that binds to CD3 comprises
a. a heavy chain variable region (VH) comprising the sequence of SEQ ID NO: 29, or a sequence having at least 80%, least 90%, at least 95%, at least 97%, or at least 99% amino acid sequence identity to the sequence of SEQ ID NO: 29;
b. and, optionally a light chain variable region (VL) comprising the sequence of SEQ ID NO: 30 or a sequence having at least 80%, at least 90%, at least 95%, at least 97%, or at least 99% amino acid sequence identity to the sequence of SEQ ID NO: 30.

31. The antibody according to any one of claims 26 to 30, wherein the antigen binding region that binds to CD3 comprises
a. a heavy chain variable region (VH) comprising the sequence of SEQ ID NO: 29, and
b. a light chain variable region (VL) comprising the sequence of SEQ ID NO: 30.

32. The antibody according to any one of claims 25 to 30, wherein said antibody has a lower human CD3ε binding affinity than an antibody having an antigen-binding region comprising a VH sequence as set forth in SEQ ID NO: 29, and a VL sequence as set forth in SEQ ID NO: 30, preferably wherein said affinity is at least 5-fold lower, such as at least 10-fold lower, e.g. at least 20-fold lower, at least 30 fold lower, at least 40 fold lower, at least 45 fold lower or such as at least 50-fold lower, such as at least 54-fold lower.

33. The antibody according to any one of claims 26 to 32, wherein said antigen binding region that binds to CD3 binds with an equilibrium dissociation constant KD within the range of 200-1000 nM, such as within the range of 300-1000 nM, within the range of 400-1000 nM, within the range of 500-1000 nM, within the range of 300-900 nM within the range of 400-900 nM, within the range of 400-700 nM, within the range of 500-900 nM, within the range of 500-800 nM, within the range of 500-700 nM, within the range of 600-1000 nM, within the range of 600-900 nM, within the range of 600-800 nM, or such as within the range of 600-700 nM.

34. The antibody according to any one of claims 26 to 31, wherein said antigen binding region that binds to CD3 binds with an equilibrium dissociation constant KD within the range of 1-100 nM, such as within the range of 5-100 nM, within the range of 10-100 nM, within the range of 1-80 nM, within the range of 1-60 nM within the range of 1-40 nM, within the range of 1-20 nM, within the range of 5-80 nM, within the range of 5-60 nM, within the range of 5-40 nM, within the range of 5-20 nM, within the range of 10-80 nM, within the range of 10-60 nM, within the range of 10-40 nM, or such as within the range of 10-20 nM.

35. The antibody according to any one of claims 26 to 34, wherein
the antigen binding region that binds to CD3 comprises a heavy chain variable (VH) region comprising a CDR1 sequence, a CDR2 sequence and a CDR3 sequence,
the heavy chain variable (VH) region, when compared to a heavy chain variable (VH) region comprising the sequence set forth in SEQ ID NO: 29, has an amino acid substitution in one of the CDR sequences, the substitution being at a position selected from the group consisting of: T31, N57, H101, G105, S110 and Y114, the positions being numbered according to the sequence of SEQ ID NO: 29; and
the wild type light chain variable (VL) region comprises the CDR1, CDR2 and CDR3 sequences set forth in SEQ ID NO: 27, GTN and SEQ ID NO: 28, respectively.

36. The antibody according to any one of claims 26 to 35 wherein the CDR1, CDR2 and CDR3 of the heavy chain variable (VH) region of the antigen binding region that binds to CD3 comprises, in total, at the most 1, 2, 3, 4 or 5 amino acid substitutions, when compared to the CDR1, CDR2 and CDR3 of the sequence set forth in SEQ ID NO: 29.

37. The antibody according to any one of claims 26 to 35, wherein the antigen binding region that binds to CD3 comprises a mutation in the VH region selected from the group consisting of: T31M, T31P, N57E, H101G, H101N, G105P, S110A, S110G, Y114M, Y114R, Y114V.

38. The antibody according to any one of claims 25 to 36, wherein the antigen-binding region capable of binding to CD3 comprises a heavy chain variable region (VH) comprising CDR1, CDR2, and CDR3 having the sequences as set forth in SEQ ID NOs: 23, 24, and 31, respectively, and a light chain variable region (VL) comprising CDR1, CDR2, and CDR3 having the sequences as set forth in SEQ ID NO: 27, the sequence GTN, and the sequence as set forth in SEQ ID NO: 28, respectively, respectively.

39. The antibody according to any one of claims 26, 27, 29 to 38, wherein the antigen-binding region capable of binding to CD3 comprises a heavy chain variable region (VH) comprising the sequence set forth in SEQ ID NO: 32 and a light chain variable region (VL) comprising the sequence set forth in SEQ ID NO: 30.

40. The antibody according to any of the preceding claims which is a bispecific antibody comprising a first antigen binding region capable of binding to human ROR2 and a second binding region capable of binding to human CD3, wherein said first antigen binding region comprises:
a heavy chain variable (VH) region CDR1, CDR2, and CDR3 having the sequences as set forth in SEQ ID NOs:3, 4, and 5, respectively, and a light chain variable (VL) region CDR1, CDR2, and CDR3 having the sequences as set forth in SEQ ID NO:7, 8 and 9, respectively;
and said second antigen binding region comprises:
a heavy chain variable (VH) region CDR1, CDR2, and CDR3 having the sequences as set forth in NOs.: 23, 24 and 25, respectively; [anti-CD3 (SP34/humanized SP34, WO2015001085 (Genmab))—VH CDR sequences], and a light chain variable region (VL) comprising the CDR1, CDR2, and CDR3 sequences of SEQ ID NO: 27, GTN and 28, respectively.

41. The antibody according to any of the preceding claims which is a bispecific antibody comprising a first antigen binding region capable of binding to human ROR2 and a second binding region capable of binding to human CD3, wherein said first antigen binding region comprises:
a heavy chain variable (VH) region CDR1, CDR2, and CDR3 having the sequences as set forth in SEQ ID NOs: 3, 4, and 5, respectively, and a light chain variable (VL) region CDR1, CDR2, and CDR3 having the sequences as set forth in SEQ ID NO: 7, 8 and 9, respectively;
and said second antigen binding region comprises:
a heavy chain variable (VH) region comprising CDR1, CDR2, and CDR3 having the sequences as set forth in SEQ ID NOs: 23, 24, and 31, respectively, and a light chain variable region (VL) comprising CDR1, CDR2, and CDR3 having the sequences as set forth in SEQ ID NO: 27, the sequence GTN, and the sequence as set forth in SEQ ID NO: 28, respectively.

42. The antibody according to any one of the preceding claims wherein said antibody comprises a first antigen binding region capable of binding to human ROR2 and a second antigen binding region capable of binding to human CD3, wherein said first antigen binding region comprises a VH region comprising the sequence as set forth in SEQ ID NO: 13, and a VL region comprising the sequence as set forth in SEQ ID NO: 19, and said second antigen binding region comprises a VH region comprising the sequence as set forth in SEQ ID NO: 29, and a VL region comprising the sequence as set forth in SEQ ID NO: 30.

43. The antibody according to any one of claims 1 to 30, 32, 33, 35 to 39 and 41, wherein said antibody comprises a first antigen binding region capable of binding to human ROR2 and a second antigen binding region capable of binding to human CD3, wherein said first antigen binding region comprises a VH region comprising the sequence as set forth in SEQ ID NO: 13, and a VL region comprising the sequence as set forth in SEQ ID NO: 19, and said second antigen binding region comprises a VH region comprising the sequence as set forth in SEQ ID NO: 32, and a VL region comprising the sequence as set forth in SEQ ID NO: 30.

44. The antibody according to any one of the preceding claims, wherein
a) the antigen-binding region(s) capable of binding to ROR2 is/are humanized, and/or
b) the antigen-binding region capable of binding to CD3, if present, is humanized.

45. The antibody according to any one of the preceding claims, wherein said antibody comprises a first and a second heavy chain constant region, each of said first and second heavy chain constant regions comprises at least a hinge region, a CH2 and CH3 region, wherein in said first heavy chain constant region at least one of the amino acids in the positions corresponding to positions selected from the group consisting of T366, L368, K370, D399, F405, Y407 and K409 in a human IgG1 heavy chain has been substituted, and in said second heavy chain constant region at least one of the amino acids in the positions corresponding to a position selected from the group consisting of T366, L368, K370, D399, F405, Y407, and K409 in a human IgG1 heavy chain has been substituted, wherein said substitutions of said first and said second heavy chains are not in the same positions, and wherein the amino acid positions in the constant regions are numbered according to Eu numbering.

46. The antibody according to any one of the preceding claims, wherein the amino acid in the position corresponding to K409 in a human IgG1 heavy chain is R in said first heavy chain, and the amino acid in the position corresponding to F405 in a human IgG1 heavy chain is L in said second heavy chain, or vice versa.

47. The antibody according to any one of the preceding claims, wherein said antibody comprises a first and a second heavy chain and wherein the first and second heavy chains are modified so that the antibody induces Fc-mediated effector function to a lesser extent relative to an identical non-modified antibody.

48. The antibody according to any one of the preceding claims, wherein the antibody comprises a first and a second heavy chain, and wherein in both the first and the second heavy chain constant region, the amino acid residues at the positions corresponding to positions L234 and L235 in a human IgG1 heavy chain according to Eu numbering are F and E, respectively.

49. The antibody according to any one of the preceding claims wherein the antibody comprises a first and a second heavy chain, and wherein in both the first and the second heavy chain constant region, the amino acid residue at the position corresponding to position D265 in a human IgG1 heavy chain according to Eu numbering is A.

50. The antibody according to any one of the preceding claims wherein the antibody comprises a first and a second heavy chain, and wherein in both the first and the second heavy chain constant regions, the amino acid residue at the position corresponding to positions L234, L235 and D265 in a human IgG1 heavy chain according to Eu numbering are F, E and A respectively.

51. The antibody according to any one of the preceding claims wherein the antibody comprises a first and a second heavy chain, and wherein in both the first and the second heavy chain constant regions, the amino acid residue at the position corresponding to positions L234, L235 and D265 in a human IgG1 heavy chain according to Eu numbering are F, E and A respectively and wherein the first heavy chain constant region further comprises a K409R substitution and the second heavy chain constant region further comprises an F405L substitution.

52. The antibody according to any one of the preceding claims wherein the antibody comprises a first and a second heavy chain constant region having the sequences as set forth in SEQ ID Nos 34 and 35, respectively, or a first and a second heavy chain constant region having the sequences as set forth in SEQ ID Nos 35 and 34, respectfully.

53. The antibody of any one of the preceding claims wherein the antibody is a bispecific antibody capable of binding to human ROR2 and human CD3 epsilon wherein
a. the first binding arm binding to ROR2 comprises:
i. The VH region having the amino acid sequence of SEQ ID NO: 13
ii. The VL region having the amino acid sequence of SEQ ID NO: 19
iii. A heavy chain constant region having the amino acid sequence of SEQ ID NO: 34 (FEAR) and
iv. A human kappa light chain constant region; and
b. the second binding arm binding to CD3 epsilon comprises:
i. The VH region having the amino acid sequence of SEQ ID NO: 29
ii. The VL region having the amino acid sequence of SEQ ID NO: 30
iii. A heavy chain constant region having the amino acid sequence of SEQ ID NO: 35 (FEAL) and
iv. A human lambda light chain constant region.

54. The antibody of any one of the preceding claims wherein the antibody is a bispecific antibody capable of binding to human ROR2 and human CD3 epsilon wherein
a. the first binding arm binding to ROR2 comprises:
i. The VH region having the amino acid sequence of SEQ ID NO: 13
ii. The VL region having the amino acid sequence of SEQ ID NO: 19
iii. A heavy chain constant region having the amino acid sequence of SEQ ID NO: 34 (FEAR) and
iv. A human kappa light chain constant region; and
b. the second binding arm binding to CD3 epsilon comprises:
i. The VH region having the amino acid sequence of SEQ ID NO: 32
ii. The VL region having the amino acid sequence of SEQ ID NO: 30
iii. A heavy chain constant region having the amino acid sequence of SEQ ID NO: 35 (FEAL) and
iv. A human lambda light chain constant region.

55. The antibody according to any one of the preceding claims, wherein said antibody comprises a lambda (λ) light chain.

56. The antibody according to any one of the preceding claims, wherein when said antibody:
a. Is capable of binding to ROR2 expressing human tumor cells such as HeLa, LCLC103-H, NCI-H1650, 786-), NCI-H23 or ZR-75-1 cells such as described in Example 6 and 10 herein
b. is capable of mediating concentration-dependent cytotoxicity of HeLa cells, when using purified PBMCs or T cells as effector cells e.g. when assayed as described in Example 11 or 12 herein,
c. is capable of mediating concentration-dependent cytotoxicity of 786-0, LCLC-103H, NCI-H23, NCH-H1650 or ZR-75-1 cells, when using purified PBMCs or T cells as effector cells e.g. when assayed as described in Example 12 herein,
d. is capable of activating T cells in vitro in the presence of HeLa, 786-0, LCLC-103H, NCI-H23, NCH-H1650 tumor cells; e.g. when assayed as described in Example 14 herein, and/or
e. is capable of inducing T cell cytokine production when using tumor cells such as HeLa and 786-0 cells as target cells e.g. when assayed as described in Example 13 herein.

57. A composition comprising an antibody as defined in any one of claims 1 to 56.

58. A pharmaceutical composition comprising an antibody as defined in any one of claims 1 to 56 and a pharmaceutically acceptable carrier.

59. The antibody as defined in any of claims 1 to 56 for use as a medicament.

60. The antibody for use as a medicament according to claim 59 for use in the treatment of a disease.

61. The antibody for use as a medicament according to claim 60, wherein the disease is cancer.

62. The antibody for use as a medicament according to claim 61, wherein the cancer is characterized by expression of ROR2 on the surface of the cancer cells.

63. The antibody for use as a medicament for use according to claim 62, wherein said expression of ROR2 is determined in cancer cells obtained from a patient.

64. The antibody for use as a medicament according to any one of claims 61 to 63 wherein the cancer is a solid tumor.

65. The antibody for use according to any one of claims 61 to 64, wherein the cancer is selected from the group comprising sarcomas, fibrosarcoma, gastro-intestinal stromal tumors, leiomyosarcoma, rhabdomyosarcoma, liposarcoma, uterine cancer, lung cancer, pancreatic cancer, renal cancer, colorectal cancer, cervical cancer and breast cancer.

66. A method of treating a disease, the method comprising administering an antibody as defined in any one of claims 1 to 56, the composition as defined in claim 57, or the pharmaceutical composition defined in claim 58, to a subject in need thereof.

67. The method according to claim 66, said method being for treatment of a cancer.

68. The method according to claim 67, wherein the cancer is selected from the group comprising sarcomas, fibrosarcoma, gastro-intestinal stromal tumors, leiomyosarcoma,
rhabdomyosarcoma, liposarcoma, uterine cancer, lung cancer, pancreatic cancer, renal cancer, colorectal cancer, cervical cancer and breast cancer.

69. A nucleic acid comprising:
a. a nucleic acid sequence encoding a heavy chain variable region sequence of an antigen-binding region capable of binding to ROR2 as defined in any one of claims 1 to 5, 8 and 9, and/or
b. a nucleic acid sequence encoding the corresponding light chain variable region sequence of said antigen-binding region capable of binding to ROR2 as defined in any one of claims 1 to 3 and 6 to 9.

70. One or more nucleic acids comprising
a. a nucleic acid sequence encoding a heavy chain sequence of an antibody comprising an antigen-binding region capable of binding to ROR2 as defined in claim 9,
b. a nucleic acid sequence encoding the corresponding light chain sequence of an antibody comprising said antigen-binding region capable of binding to ROR2 as defined in claim 9.

71. One or more nucleic acids comprising
a. a nucleic acid sequence encoding a heavy chain sequence of an antibody comprising an antigen-binding region capable of binding to ROR2 as set forth in SEQ ID NO:13, and/or
b. a nucleic acid sequence encoding a light chain sequence of an antibody comprising an antigen-binding region capable of binding to ROR2 as set forth in SEQ ID NO:19.

72. A nucleic acid, or one or more nucleic acids, according to any one of claims 69 to 71, wherein said nucleic acid is RNA or DNA.

73. A nucleic acid, or one or more nucleic acids, according to any one of claims 69 to 72 for use in expression in mammalian cells.

74. An expression vector comprising
a) a nucleic acid sequence encoding a heavy chain sequence of an antibody comprising an antigen-binding region capable of binding to ROR2 as defined in any one of claims 69 to 73, and/or
b) a nucleic acid sequence encoding a light chain sequence of an antibody comprising an antigen-binding region capable of binding to ROR2 as defined in any one of claims 69 to 73.

75. The expression vector according to claim 74, further comprising
a. a nucleic acid sequence encoding a heavy chain sequence of an antibody comprising an antigen-binding region capable of binding to CD3 as defined in any one of claims 28 to 31 and 35 to 39; and/or
b. a nucleic acid sequence encoding a light chain sequence of an antibody comprising an antigen-binding region capable of binding to CD3 as defined in any one of claims 28 to 31 and 35 to 39.

76. A cell comprising a nucleic acid, or comprising one or more nucleic acids, as defined in any one of claims 69 to 73 or an expression vector as defined in claims 74 or 75.

77. The cell according to claim 76, wherein said cell is of human origin, such as a human embryonic kidney (HEK) cell, or is of rodent origin, such as a Chinese hamster ovary cell (CHO cell).

78. A method for producing an antibody capable of binding to both ROR2 and CD3 in accordance with any one of claims 1 to 56, comprising the steps of:
a. providing an antibody capable of binding to ROR2, said antibody comprising an antigen-binding region capable of binding to ROR2 as defined in any one of claims 1 to 56;
b. providing an antibody capable of binding to CD3, said antibody comprising an antigen-binding region capable of binding to CD3 as defined in any one of claims 26 to 56;
c. incubating said antibody capable of binding to ROR2 together with said antibody capable of binding to CD3 under reducing conditions sufficient to allow cysteines in the hinge region to undergo disulfide-bond isomerization, and
d. obtaining said antibody capable of binding to ROR2 and CD3.

79. The method for producing an antibody capable of binding to both ROR2 and CD3, in accordance with claim 78, wherein the steps a) and/or b) comprise:
providing cells containing expression vectors for producing said antibody or said antibodies; and
allowing the cells to produce said antibody or said antibodies and subsequently,
obtaining said antibody or said antibodies, thereby providing said antibody or said antibodies.

80. A kit-of-parts, such as a kit for use as a companion diagnostic/for identifying within a population of patients those patients which have a propensity to respond to treatment with an antibody as defined in any one of claims 1 to 56, comprising an antibody as defined in any one of claims 1 to 56; and instructions for use of said kit.

81. An anti-idiotypic antibody, which binds to the antigen-binding region capable of binding to ROR2 as defined in any one of claims 1 to 56.


SEQUENCE LISTING (51 sequences)

SEQ ID NO: 1 - protein, 943 aa, Homo sapiens
  SITE (1)..(943): Human ROR2
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNVVCLLGVVTKDQPLSMIFSYCSHGDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYVGPKQKAPPFPQPQFIPMKGQIRPMVPPPQLYVPVNGYQPVPAYGAYLPNFYPVQIPMQMAPQQVPPQMVPKPSSHHSGSGSTSTGYVTTAPSNTSMADRAALLSEGADDTQNAPEDGAQSTVQEAEEEEEGSVPETELLGDCDTLQVDEAQVQLEA

SEQ ID NO: 2 - protein, 116 aa, Artificial Sequence
  Synthetic ROR2-A_VH
QSVEESGGRLVTPGTPLTLTCTVSGFSLSSYSMSWVRQAPGKGLEWIGVIYVNSKTWYANWAKGRFTISKTSTTVDLKITSPTTEDTATYFCARGDAGYTTNSWLWGPGTLVTVSS

SEQ ID NO: 3 - protein, 8 aa, Artificial Sequence
  Synthetic ROR2-A_VH-CDR1
GFSLSSYS

SEQ ID NO: 4 - protein, 7 aa, Artificial Sequence
  Synthetic ROR2-A_VH-CDR2
IYVNSKT

SEQ ID NO: 5 - protein, 13 aa, Artificial Sequence
  Synthetic ROR2-A_VH-CDR3
ARGDAGYTTNSWL

SEQ ID NO: 6 - protein, 108 aa, Artificial Sequence
  Synthetic ROR2-A_VL
AFELTQTPASVSEPVGGTVTIKCQASESIDNYCSWYQQKPGQPPKLLIYQAFKLASGVPSRFRGSGSGTQFTLTISDLESDDAATYYCQSYSGISTTAFGGGTEVVVK

SEQ ID NO: 7 - protein, 6 aa, Artificial Sequence
  Synthetic ROR2-A_VL-CDR1
ESIDNY

SEQ ID NO: 8 - protein, 3 aa, Artificial Sequence
  Synthetic ROR2-A_VL-CDR2
QAF

SEQ ID NO: 9 - protein, 10 aa, Artificial Sequence
  Synthetic ROR2-A_VL-CDR3
QSYSGISTTA

SEQ ID NO: 10 - protein, 116 aa, Artificial Sequence
  Synthetic ROR2-A_ HC1
QSVLESGGGLVQPGGSLRLSCTVSGFSLSSYSMSWVRQAPGKGLEWIGVIYVNSKTWYANWAKGRFTISKTSTTVDLKITSPTAEDTATYFCARGDAGYTTNSWLWGQGTLVTVSS

SEQ ID NO: 11 - protein, 116 aa, Artificial Sequence
  Synthetic ROR2-A_ HC2
QSVLESGGGLVQPGGSLRLSCTVSGFSLSSYSMSWVRQAPGKGLEWIGVIYVNSKTWYANWAKGRFTISKTSTTVYLQMNSLRAEDTATYFCARGDAGYTTNSWLWGQGTLVTVSS

SEQ ID NO: 12 - protein, 117 aa, Artificial Sequence
  Synthetic ROR2-A_ HC3
EVQLLESGGGLVQPGGSLRLSCTVSGFSLSSYSMSWVRQAPGKGLEWIGVIYVNSKTWYANWAKGRFTISKTSTTVYLQMNSLRAEDTATYYCARGDAGYTTNSWLWGQGTLVTVSS

SEQ ID NO: 13 - protein, 119 aa, Artificial Sequence
  Synthetic ROR2-A_ HC4
EVQLLESGGGLVQPGGSLRLSCTVSGFSLSSYSMSWVRQAPGKGLEWIGVIYVNSKTWYANWAKGRFTISKDNSKNTVYLQMNSLRAEDTATYYCARGDAGYTTNSWLWGQGTLVTVSS

SEQ ID NO: 14 - protein, 119 aa, Artificial Sequence
  Synthetic ROR2-A_ HC5
EVQLLESGGGLVQPGGSLRLSCAASGFSLSSYSMSWVRQAPGKGLEWIGVIYVNSKTWYANWAKGRFTISKDNSKNTVYLQMNSLRAEDTATYYCARGDAGYTTNSWLWGQGTLVTVSS

SEQ ID NO: 15 - protein, 119 aa, Artificial Sequence
  Synthetic ROR2-A_ HC6
EVQLLESGGGLVQPGGSLRLSCTVSGFSLSSYSMSWVRQAPGKGLEWIGVIYVNSKTWYADSVKGRFTISKDNSKNTVYLQMNSLRAEDTATYYCARGDAGYTTNSWLWGQGTLVTVSS

SEQ ID NO: 16 - protein, 119 aa, Artificial Sequence
  Synthetic ROR2-A_ HC7
EVQLLESGGGLVQPGGSLRLSCAASGFSLSSYSMSWVRQAPGKGLEWIGVIYVNSKTWYADSVKGRFTISKDNSKNTVYLQMNSLRAEDTATYYCARGDAGYTTNSWLWGQGTLVTVSS

SEQ ID NO: 17 - protein, 108 aa, Artificial Sequence
  Synthetic ROR2-A_ LC1
AFELTQSPSSLSASVGDRVTITCQASESIDNYCSWYQQKPGKAPKLLIYQAFKLASGVPSRFRGSGSGTDFTLTISSLQSEDFATYYCQSYSGISTTAFGGGTEVVIK

SEQ ID NO: 18 - protein, 108 aa, Artificial Sequence
  Synthetic ROR2-A_ LC2
AFELTQSPSSLSASVGDRVTITCQASESIDNYLSWYQQKPGKAPKLLIYQAFKLASGVPSRFRGSGSGTDFTLTISSLQSEDFATYYCQSYSGISTTAFGGGTEVVIK

SEQ ID NO: 19 - protein, 108 aa, Artificial Sequence
  Synthetic ROR2-A_ LC3
AFELTQSPSSLSASVGDRVTITCQASESIDNYLSWYQQKPGKAPKLLIYQAFKLASGVPSRFRGSGSGTDFTLTISSLQPEDFATYYCQSYSGISTTAFGGGTKVEIK

SEQ ID NO: 20 - protein, 108 aa, Artificial Sequence
  Synthetic ROR2-A_ LC4
DIQLTQSPSSLSASVGDRVTITCQASESIDNYLSWYQQKPGKAPKLLIYQAFKLASGVPSRFRGSGSGTDFTLTISSLQPEDFATYYCQSYSGISTTAFGGGTKVEIK

SEQ ID NO: 21 - protein, 186 aa, Homo sapiens
  SITE (1)..(186): human CD3? (epsilon)
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 22 - protein, 125 aa, Mus musculus
  SITE (1)..(125): CD3-SP34_VH WT
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 23 - protein, 8 aa, Mus musculus
  SITE (1)..(8): CD3-SP34_VH-CDR1
GFTFNTYA

SEQ ID NO: 24 - protein, 10 aa, Mus musculus
  SITE (1)..(10): CD3-SP34_VH-CDR2
IRSKYNNYAT

SEQ ID NO: 25 - protein, 16 aa, Mus musculus
  SITE (1)..(16): CD3-SP34_VH-CDR3
VRHGNFGNSYVSWFAY

SEQ ID NO: 26 - protein, 109 aa, Mus musculus
  SITE (1)..(109): CD3-SP34_VL WT
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 27 - protein, 9 aa, Mus musculus
  SITE (1)..(9): CD3-SP34_VL-CDR1
TGAVTTSNY

SEQ ID NO: 28 - protein, 9 aa, Mus musculus
  SITE (1)..(9): CD3-SP34_VL-CDR3
ALWYSNLWV

SEQ ID NO: 29 - protein, 125 aa, Artificial Sequence
  Synthetic huCD3-H1L1_VH
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRHGNFGNSYVSWFAYWGQGTLVTVSS

SEQ ID NO: 30 - protein, 109 aa, Artificial Sequence
  Synthetic huCD3-H1L1_VL
QAVVTQEPSFSVSPGGTVTLTCRSSTGAVTTSNYANWVQQTPGQAFRGLIGGTNKRAPGVPARFSGSLIGDKAALTITGAQADDESIYFCALWYSNLWVFGGGTKLTVL

SEQ ID NO: 31 - protein, 16 aa, Artificial Sequence
  Synthetic huCD3-H1L1-H101G_VH CDR3
VRGGNFGNSYVSWFAY

SEQ ID NO: 32 - protein, 125 aa, Artificial Sequence
  Synthetic huCD3-H1L1-H101G_VH
EVKLVESGGGLVQPGGSLRLSCAASGFTFNTYAMNWVRQAPGKGLEWVARIRSKYNNYATYYADSVKDRFTISRDDSKSSLYLQMNNLKTEDTAMYYCVRGGNFGNSYVSWFAYWGQGTLVTVSS

SEQ ID NO: 33 - protein, 330 aa, Homo sapiens
  SITE (1)..(330): Human IgG1-Fc
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 330 aa, Artificial Sequence
  Synthetic Human IgG1-Fc-FEAR
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 35 - protein, 330 aa, Artificial Sequence
  Synthetic Human IgG1-Fc-FEAL
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 36 - protein, 107 aa, Homo sapiens [sequence omitted]

SEQ ID NO: 37 - protein, 106 aa, Homo sapiens
  SITE (1)..(106): Human lambda LC
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 330 aa, Artificial Sequence
  Synthetic IgG1-F405L
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 39 - protein, 942 aa, Macaca fascicularis
  SITE (1)..(942): cynomolgus monkey (Macaca fascicularis) ROR2
MARGSALPRRPLLCIPAVWAAAALLLSVSRTSGEVEVPDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRAPKPRELCRDECEVLESDLCRQEYTIARSNPLILMRLQLPKCEALPMPESPDAANCMRIGIPAERLGRYHQCYNGSGTDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNIVCLLGVVTKDQPLSMIFSYCSHSDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYMGPKQKAPPFPQPQFIPMKGQIRPMVPPPQLYIPVNGYQPVPAYGAYLPNFYPVQIPMQMAPQQVPPQMVPKPSSHHSGSGSTSTGYVTTAPSNTSVADRAALLSEGTEDAQNAPEDGAQSPVQEAEEEEGSVPETELLGDSDTLQVDEAQVQLEA

SEQ ID NO: 40 - protein, 937 aa, Homo sapiens
  SITE (1)..(937): Human ROR1
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 41 - protein, 942 aa, Homo sapiens
  SITE (1)..(942): ROR2mf-T322M
MARGSALPRRPLLCIPAVWAAAALLLSVSRTSGEVEVPDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRAPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCSPRDSSKMGILYILVPSIAIPLVIACLFFLVCMCRNKQKASASTPQRRQLMASPSQDMEMPLINQHKQAKLKEISLSAVRFMEELGEDRFGKVYKGHLFGPAPGEQTQAVAIKTLKDKAEGPLREEFRHEAMLRARLQHPNIVCLLGVVTKDQPLSMIFSYCSHSDLHEFLVMRSPHSDVGSTDDDRTVKSALEPPDFVHLVAQIAAGMEYLSSHHVVHKDLATRNVLVYDKLNVKISDLGLFREVYAADYYKLLGNSLLPIRWMAPEAIMYGKFSIDSDIWSYGVVLWEVFSYGLQPYCGYSNQDVVEMIRNRQVLPCPDDCPAWVYALMIECWNEFPSRRPRFKDIHSRLRAWGNLSNYNSSAQTSGASNTTQTSSLSTSPVSNVSNARYMGPKQKAPPFPQPQFIPMKGQIRPMVPPPQLYIPVNGYQPVPAYGAYLPNFYPVQIPMQMAPQQVPPQMVPKPSSHHSGSGSTSTGYVTTAPSNTSVADRAALLSEGTEDAQNAPEDGAQSPVQEAEEEEGSVPETELLGDSDTLQVDEAQVQLEA

SEQ ID NO: 42 - protein, 929 aa, Artificial Sequence
  Synthetic ROR112
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNCYNGSGMDYRGTASTTKSGHQCQPWALQHPHSHHLSSTDFPELGGGHAYCRNPGGQMEGPWCFTQNKNVRMELCDVPSCSPRDSSKMGILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 43 - protein, 936 aa, Artificial Sequence
  Synthetic ROR121
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 44 - protein, 936 aa, Artificial Sequence
  Synthetic ROR122
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIGIPAERLGRYHQCYNGSGMDYRGTASTTKSGHQCQPWALQHPHSHHLSSTDFPELGGGHAYCRNPGGQMEGPWCFTQNKNVRMELCDVPSCDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 45 - protein, 928 aa, Artificial Sequence
  Synthetic ROR211
MARGSALPRRPLLCIPAVWAAAALLLSVSRTSGEVEVLDPNDPLGPLDGQDGPIPTLKGYFLNFLEPVNNITIVQGQTAILHCKVAGNPPPNVRWLKNDAPVVQEPRRIIIRKTEYGSRLRIQDLDTTDTGYYQCVATVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 46 - protein, 927 aa, Artificial Sequence
  Synthetic ROR221
MHRPRRRGTRPPLLALLAALLLAARGAAAQETELSVSAELVPTLKGYFLNFLEPVNNITIVQGQTAILHCKVAGNPPPNVRWLKNDAPVVQEPRRIIIRKTEYGSRLRIQDLDTTDTGYYQCVATNGMKTITATGVLFVRLGPTHSPNHNFQDDYHEDGFCQPYRGIACARFIGNRTIYVDSLQMQGEIENRITAAFTMIGTSTHLSDQCSQFAIPSFCHFVFPLCDARSRTPKPRELCRDECEVLESDLCRQEYTIARSNPLILMRLQLPKCEALPMPESPDAANCMRIIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 47 - protein, 127 aa, Homo sapiens
  SITE (1)..(127): b12_VH
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 48 - protein, 108 aa, Homo sapiens
  SITE (1)..(108): b12_VL
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 49 - protein, 330 aa, Artificial Sequence
  Synthetic IgG1-K409R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 50 - protein, 330 aa, Artificial Sequence
  Synthetic IgG1-FEA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 255 aa, Artificial Sequence
  Synthetic CD3E27-GSKa
QDGNEEMGGITQTPYKVSISGTTVILTGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE